SEQUENCE LISTING (40 sequences)

SEQ ID NO: 1 - DNA, 850 bp, Homo sapiens
  CDS (1)..(831)
atgggcttgttagagtgctgtgcaagatgtctggtaggggccccctttgcttccctggtggccactggattgtgtttctttggggtggcactgttctgtggctgtggacatgaagccctcactggcacagaaaagctaattgagacctatttctccaaaaactaccaagactatgagtatctcatcaatgtgatccatgccttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaagtttgtgggcatcacctatgccctgaccgttgtgtggctcctggtgtttgcctgctctgctgtgcccgtgtacatttacttcaacacctggaccacctgcgactctattgccttccccagcaagacctctgccagtataggcagtctctgtgctgacgccagaatgtatggtgttctcccatggaatgctttccctggcaaggtttgtggctccaaccttctgtccatctgcaaaacagctgagttccaaatgaccttccacctgtttattgctgcatttgtgggggctgcagctacactggtttccctgctcaccttcatgattgctgccacttacaactttgccgtccttaaactcatgggccgaggcaccaagttctgatacactggtttccctg

SEQ ID NO: 2 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCDSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 3 - DNA, 729 bp, Homo sapiens
  CDS (1)..(726)
atgggcttgttagagtgctgtgcaagatgtctggtaggggccccctttgcttccctggtggccactggattgtgtttctttggggtggcactgttctgtggctgtggacatgaagccctcactggcacagaaaagctaattgagacctatttctccaaaaactaccaagactatgagtatctcatcaatgtgatccatgccttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacgtttgtgggcatcacctatgccctgaccgttgtgtggctcctggtgtttgcctgctctgctgtgcccgtgtacatttacttcaacacctggaccacctgcgactctattgccttccccagcaagacctctgccagtataggcagtctctgtgctgacgccagaatgtatggtgttctcccatggaatgctttccctggcaaggtttgtggctccaaccttctgtccatctgcaaaacagctgagttccaaatgaccttccacctgtttattgctgcatttgtgggggctgcagctacactggtttccctgctcaccttcatgattgctgccacttacaactttgccgtccttaaactcatgggccgaggcaccaagttctga

SEQ ID NO: 4 - protein, 242 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATFVGITYALTVVWLLVFACSAVPVYIYFNTWTTCDSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 5 - DNA, 216 bp, Homo sapiens
  CDS (1)..(216)
atgtatggtgttctcccatggaatgctttccctggcaaggtttgtggctccaaccttctgtccatctgcaaaacagctgagttccaaatgaccttccacctgtttattgctgcatttgtgggggctgcagctacactggtttccctgctcaccttcatgattgctgccacttacaactttgccgtccttaaactcatgggccgaggcaccaagttc

SEQ ID NO: 6 - protein, 72 aa, Homo sapiens
MYGVLPWNAFPGKVCGSNLLSICKTAEFQMTFHLFIAAFVGAAATLVSLLTFMIAATYNFAVLKLMGRGTKF

SEQ ID NO: 7 - DNA, 129 bp, Homo sapiens
  CDS (1)..(129)
atgaccttccacctgtttattgctgcatttgtgggggctgcagctacactggtttccctgctcaccttcatgattgctgccacttacaactttgccgtccttaaactcatgggccgaggcaccaagttc

SEQ ID NO: 8 - protein, 43 aa, Homo sapiens
MTFHLFIAAFVGAAATLVSLLTFMIAATYNFAVLKLMGRGTKF

SEQ ID NO: 9 - DNA, 235 bp, Homo sapiens
  CDS (11)..(226)
gagctccaccatgtacggtgttctcccttggaacgctttccctggcaaggtttgtggctccaaccttctgtccatctgcaaaacagccgagttccaaatgaccttccacctgtttattgctgcgtttgtgggtgctgcggccacactagtttccctgctcaccttcatgattgctgccacttacaacttcgccgtccttaaactcatgggccgaggcaccaagttctgaccgcgg

SEQ ID NO: 10 - protein, 72 aa, Homo sapiens; identical to SEQ ID NO: 6

SEQ ID NO: 11 - DNA, 253 bp, Homo sapiens
  CDS (11)..(244)
gagctccaccatgtacggtgttctcccttggaacgctttccctggcaaggtttgtggctccaaccttctgtccatctgcaaaacagccgagttccaaatgaccttccacctgtttattgctgcgtttgtgggtgctgcggccacactagtttccctgctcaccttcatgattgctgccacttacaacttcgccgtccttaaactcatgggccgaggcaccaagttccatcatcaccatcaccattgaccgcgg

SEQ ID NO: 12 - protein, 78 aa, Homo sapiens
MYGVLPWNAFPGKVCGSNLLSICKTAEFQMTFHLFIAAFVGAAATLVSLLTFMIAATYNFAVLKLMGRGTKFHHHHHH

SEQ ID NO: 13 - DNA, 148 bp, Homo sapiens
  CDS (11)..(139)
gagctccaccatgaccttccacctgtttattgctgcgtttgtgggtgctgcggccacactagtttccctgctcaccttcatgattgctgccacttacaacttcgccgtccttaaactcatgggccgaggcaccaagttctgaccgcgg

SEQ ID NO: 14 - protein, 43 aa, Homo sapiens; identical to SEQ ID NO: 8

SEQ ID NO: 15 - DNA, 166 bp, Homo sapiens
  CDS (11)..(157)
gagctccaccatgaccttccacctgtttattgctgcgtttgtgggtgctgcggccacactagtttccctgctcaccttcatgattgctgccacttacaacttcgccgtccttaaactcatgggccgaggcaccaagttccatcatcaccatcaccattgaccgcgg

SEQ ID NO: 16 - protein, 49 aa, Homo sapiens
MTFHLFIAAFVGAAATLVSLLTFMIAATYNFAVLKLMGRGTKFHHHHHH

SEQ ID NO: 17 - DNA, 161 bp, Murinae gen. sp.
  CDS (11)..(124)
gagctccaccatgtatgttccaaatgaccttccacctgtttattgctgcgtttgtgggtgctgcggccacactagtttccctgctcaccttcatgattgctgccacttacaacttcgccgtccttaaactcatgggccgaggcaccaagttctgaccgcgg

SEQ ID NO: 18 - protein, 38 aa, Murinae gen. sp.
MYVPNDLPPVYCCVCGCCGHTSFPAHLHDCCHLQLRRP

SEQ ID NO: 19 - DNA, 27 bp, Unknown
  primer
cgggatcctcagagtgccaaagacatg

SEQ ID NO: 20 - DNA, 27 bp, Unknown
  primer
tttccgcgggaacttggtgcctcggcc

SEQ ID NO: 21 - DNA, 30 bp, Unknown
  primer
tttccgcggtcagaacttggtgcctcggcc

SEQ ID NO: 22 - DNA, 48 bp, Unknown
  primer
tttccgcggtcaatggtgatggtgatgatggaacttggtgcctcggcc

SEQ ID NO: 23 - DNA, 26 bp, Unknown
  primer
tttccgcggaggacggcgaagttgta

SEQ ID NO: 24 - DNA, 26 bp, Unknown
  primer
cggaattctcagagtgccaaagacat

SEQ ID NO: 25 - DNA, 42 bp, Unknown
  primer
tccccgcgggaacttggtgcctcggcccatgagtctaaggac

SEQ ID NO: 26 - DNA, 42 bp, Unknown
  primer
tccccgcgggaacctggtgcctcggcccatgagtttaaggac

SEQ ID NO: 27 - DNA, 42 bp, Unknown
  primer
tccccgcgggaacctggtgcctcggcccatgagtctaaggac

SEQ ID NO: 28 - DNA, 26 bp, Unknown
  primer
cggaattctcagagtgccaaagacat

SEQ ID NO: 29 - DNA, 33 bp, Unknown
  primer
tttccgcgggaacttggtgcctttgcccatgag

SEQ ID NO: 30 - DNA, 35 bp, Unknown
  primer
cctcagagtgccaaagacttgggcttgttagagtg

SEQ ID NO: 31 - DNA, 33 bp, Unknown
  primer
ctgcgctgatgccagattgtatggtgttctccc

SEQ ID NO: 32 - DNA, 39 bp, Unknown
  primer
ctgcgctgatgccagattgtatgttccaaatgaccttcc

SEQ ID NO: 33 - DNA, 37 bp, Unknown
  primer
ctgcaaaacagctgagttccaattgaccttccacctg

SEQ ID NO: 34 - DNA, 50 bp, Unknown
  primer
tcgagagctccaccatgtacggtgttctcccttggaacgctttccctggc

SEQ ID NO: 35 - DNA, 30 bp, Unknown
  primer
tcgagagctccaccatgaccttccacctgt

SEQ ID NO: 36 - RNA, 13 nt, Unknown
  Consensus sequence
gccgccrccaugg

SEQ ID NO: 37 - DNA, 35 bp, Unknown
  primer
cactctaacaagcccaagtctttggcactctgagg

SEQ ID NO: 38 - DNA, 33 bp, Unknown
  primer
gggagaacaccatacaatctggcatcagcgcag

SEQ ID NO: 39 - DNA, 39 bp, Unknown
  primer
ggaaggtcatttggaacatacaatctggcatcagcgcag

SEQ ID NO: 40 - DNA, 37 bp, Unknown
  primer
caggtggaaggtcaattggaactcagctgttttgcag

(19) United States
(12) Patent Application Publication
(10) Pub. No.: US 2006/0173168 A1
(43) Pub. Date: Aug. 3, 2006

(54) Bioactive peptides and unique ires elements from myelin proteolipid protein plp/dm20

(75) Inventors: Leon Carlock, Bloomfield Hills, MI (US); Maria Cypher, Madison Heights, MI (US)

Correspondence Address: MCKENNA LONG & ALDRIDGE LLP, 1900 K STREET, NW, WASHINGTON, DC, 20006, US

(73) Assignee: Wayne State University, Detroit, MI (US)

(21) Appl. No.: 10/539,634

(22) PCT Filed: Dec. 16, 2003
(86) PCT No.: PCT/US03/39873

§ 371 Date: Dec. 9, 2005

Related U.S. Application Data
(60) Provisional application No. 60/433,573, filed on Dec. 16, 2002.

(51) Int. Cl. A61K 38/17; C07K 14/775; C07H 21/04; C12P 21/06

(52) U.S. Cl. 530/359; 435/69.1; 435/320.1; 435/325; 536/23.5; 514/12

(57) ABSTRACT

Three novel low molecular weight (LMW) polypeptide fragments of a proteolipid protein human PLP/DM20 are designated PIRP-M, PIRP-L and PIRP-J, and are growth factors for oligodendrocytes with anti-apoptotic activity. They are encoded by mRNA from an IRES. Fusion polypeptides of such a LMW polypeptide, DNA encoding the LMW polypeptide and fusion polypeptide, expression vectors comprising such DNA, and cells expressing such polypeptides, or pharmaceutical compositions thereof, are useful for stimulating neural stem cell differentiation, maturation along the oligodendrocytic pathway and proliferation of oligodendrocytes or precursors. These compositions can protect oligodendrocytes (and nonneural cells) from apoptotic death. Thus, the present composition is used to treat a disease or condition in which such differentiation, maturation and proliferation or inhibition of cell death, including remyelination or stimulation of oligodendroglia or Schwann cells, is desirable. Disorders include multiple sclerosis, trauma with Parkinson's-like symptoms, hypoxic ischerriia and spinal cord trauma.

### BACKGROUND OF THE INVENTION

1. Field of the Invention

The present invention in the fields of molecular and cellular biology and medicine relates to (1) a novel Internal Ribosome Entry Site (IRES) present in the mRNA encoding the proteolipid proteins PLP/DM20, and (2) two novel “Proteolipid IRES Proteins” (PIRPs), PIRP-M and PIRP-L, encoded by this mRNA the synthesis of which is initiated at such IRES sites. This generates novel low molecular weight polypeptide fragments with a number of useful properties, including growth factor and anti-apoptotic activity.

2. Description of the Background Art

Proteolipid and Lipophilin Proteins: PLP/DM20

“Proteolipids” have been defined as a ubiquitous type of membrane lipoprotein soluble in chloroform/methanol and insoluble in water. The term proteolipid is commonly used to describe any plant, animal, or bacterial membrane protein that is soluble in a 2:1 (v/v) chloroform/methnol mixture. In animal tissues, the highest concentration of proteolipids is found in the myelin fraction of the central nervous system (CNS) white matter [223]. Early studies identified two classes of proteolipids: the Folch-Lees (PLP and DM20 proteins) and Wolfgram proteolipids (2′-3′ cyclic nucleotide phosphodiesterase [CNP] and other proteins) [224;225;1]. Currently, the term “myelin proteolipids” is used exclusively for the PLP and DM20 proteins.

PLP/DM20-related proteins a distinct from the other CNS proteolipid proteins based on sequence homology. The PLP/DM20-related proteins were collectively termed “lipophilins” [226], a family whose members are encoded by at least three genes in terrestrial vertebrates (PLP, M6a, and M6b), which give rise to a number of proteins via alternative splicing. Members of the lipophilin family share structural characteristics including low molecular weight (20-30 kDa), four putative transmembrane domains (TMD), localization of the N- and C termini to the cytoplasm, and strong interaction with membrane lipids [226-228]. The PLP/DM20 proteins comprise˜50% of the total protein in the CNS myelin [229]. The myelin sheath is a highly ordered spiral structure formed by specialized extensions of oligodendrocyte plasma membrane which wraps around neighboring axons [1). PLP (277 amino acids, 29.9 kDa), and DM20 (242 amino acids, 26.5 kDa) are produced from a single gene by alternative splicing [10,14]. DM20 differs from the PLP isoform by an internal deletion of 35 amino acids, Val¹¹⁶ through Lys¹⁵⁰ [35,14]. The hydrophobicity profiles of PLP/DM20 suggested a pattern of alternating hydrophobic and hydrophilic domains, which is characteristic of integral membrane proteins (See FIG. 1a). PLP/DM20 proteins are also present in the membranes of the endoplasmic reticulum (ER), Golgi complex (GC), Golgi vesicles, and myelin sheath [40;230;231;37]. The hydrophilic N-terminus, intracellular loop, and C-terminus of PLP/DM20 are in the cytoplasm [32-42].

PLP/DM20 undergo several cotranslational and posttranslational modifications. Met¹ is removed cotranslationally, so that Gly² is the first amino acid of both mature proteins [46, 47)]. (The sequences shown herein designate Met¹ as “0” and number Gly² as #1). Two disulfide bridges are present in the second extracellular loop and a number of covalently bound fatty acids on 4-6 cytoplasmic Cys residues [41]. Fatty acid composition (i.e., palmitate, oleate, and stearate) and the extent of PLP/DM20 acylation are ontogenetically and phylogenetically conserved in amphibians, birds, and mammals [48, 491. Indirect evidence exists for heteromeric complexes composed of the two proteolipid protein isoforms [232-234]

The 35 amino acid PLP-specific sequence (located in the intracellular loop) and numbered from residues 116-150 in SEQ ID NO:2, confers several unique structural features to the protein. The cluster of positive charges in the PLP-specific sequence increases affinity for the negatively charged lipids, which might be important for protein targeting to specialized membrane domains [235;236]

This sequence also contains binding sites for inositol hexaphosphate (IP₆) and the C-terminus of αᵥ-integrin, which is thought to regulate PLP-mediated signal transduction pathways [237;238]. Furthermore, the PLP Asp149 residue in the PLP-specific sequence interacts with dicyclohexylcarbodiimide (DCCD), which binds to dicarboxylic amino acid residues present in proton channels from a variety of species [240;241].

Structure of the PLP Gene

The PLP gene is located on the X chromosome (Xq13-Xq22 in humans) and spans a genomic region of˜17-20 kb (17 kb in humans). Initial analyses identified seven exons and six introns, with intron 1 being the largest (˜6-8 kb) ([3-9].

The coding specificity of the various exons is as follows:

| EXON(s) | Encodes: |
|---|---|
| 1 | 5′UTR, Met¹ codon, first base of the Gly² codon |
| 2, 3, 4 | TMD with flanking hydrophilic sequences |
| 5 | regions associated with growth factor activity |
| 6 | Most of TMD IV. |
| 7 | Hydrophilic C-terminus and 3′ UTR. |

[Refs: 3, 4, 14, 40, 106}

An additional short exon (exon 1.1) has been identified within the intron 1 sequence. This exon encodes an alternative start codon and a 12 amino acid leader sequence, which may be responsible for the soma restricted subcellular localization of the srPLP and srDM20 protein isoforms [15].

The PLP gene displays considerable evolutionary conservation. The open reading frame (ORF) of the chicken, rabbit, dog, pig, cow, mouse, rat, and human genes exhibit >95% amino acid sequence identity. The rat, mouse, and human amino acid sequences are 100% identical, the dog and rabbit sequences differ by 1 residue, and the cow sequence differs by 2. In mammals, the sequence conservation extends to codon wobble positions and non-coding sequences. In the 831 nucleotides (nt) encoding the 277 amino acid PLP protein, 11 nt differences are found between mouse and rat mRNAs, and 25 nt differences are found between mouse and human sequences, and the 5′ and 3′ UTRs are >90% identical and intron 3 sequences are ˜78% identical ]4,6-9,243]. The major transcription initiation sites in the mouse, rat, pig, baboon, and human genes map 147-160 nt upstream of Met¹ [4,8]. Several alternative polyadenylation signals are used in mammals, giving rise to 3200-3500 nt, 2400 nt, and 1500-1600 nt transcripts in most species [3-5, 8, 9]

A number of transcription factors are thought to actively regulate myelin gene synthesis. For the PLP gene, a variety of zinc finger transcription factors (CREB, SP1, MyT1), nuclear hormone receptor proteins (PPAR) and homeodomain proteins (GTX) potentially bind proximal and distal promoter sequences. In many cases, these transcriptional regulators display preferential expression during OL differentiation and myelination and appear to temporally regulate gene expression[16-20]. Expression of the PLP gene begins as early as embryonic day 9 (E9), nearly a week before the first myelinated axons are detected in the brainstem. In rodents, the DM20 mRNA is detected in OL progenitors as well as undifferentiated neuroepithelial progenitors and possibly neuronal progenitors. Little or no PLP mRNA or protein is detected during this phase of development. In contrast, differentiation and myelination stimulates a large increase in myelin-specific lipids and membrane-associated proteins. At this time, the isoform expression profile reverses with the PLP mRNA becoming preeminent (the ratio of PLP to DM20 about 2:1) and the PLP/DM20 genes accumulating to about 3% of total brain mRNA or 10% of the total mRNA in OLs [2,21-24,199].

In addition to temporal expression differences during CNS development, DM20 mRNA is found in peripheral nervous system (PNS) Schwann cells, thymus, cardiomyocytes, spleen, lymph nodes and testes with little or no detectable PLP transcript[25-30]. This implies that the DM20 protein/mRNA functions in cells other than CNS cells and undifferentiated oligodendrocytes (OL); whereas, the PLP protein/mRNA is needed for terminally differentiated OL and normal myelin function.

Putative Function of the PLP/DM20 proteins.

Sequence differences and variations in the expression patterns as well as the inability of DM20 protein to fully compensate for the absence of PLP, suggest that these proteins have independent functions. It has been proposed that PLP provides structural stability to CNS myelin [59-61;228;244;246;247; displays proton channel activity[239-241]; regulates endo- and exocytosis by interacting with IP₆ [237,251], and facilitates signal transduction between the extracellular matrix (ECM) and intracellular cytoskeleton by interacting with the integrin/calreticulin complexes [238]. In contrast, it has been proposed that the DM20 protein isoform modulates the trafficking of PLP and other molecules through the secretory pathway and acts as a developmental regulator in neural and nonneural tissues [232;233;31;196;104-106;108].

“Natural” Mutations in the PLP/DM20 gene

The myelin diseases associated with PLP gene mutations are a heterologous group of neurological diseases with a wide spectrum of symptoms in animals and humans. In general, PLP gene mutations are detected as myelin deficiencies which result from the breakdown of myelin after formation (demyelination) or the failure to synthesize myelin during development (hypomyelination or dysmyelination). Furthermore, these myelin diseases are invariably associated with a variety of abnormalities in glial cell structure and function [52-55].

Because the PLP gene is on the X-chromosome, these diseases are maternally transmitted and are expressed in (hemizygous) males and homozygous females (the latter being very rare, since most affected males are unable to breed). In contrast, heterozygous females are generally asymptomatic due to random X inactivation and the selective loss of cells expressing the mutant protein. Mutations that result in a mild phenotype are more likely to cause symptoms in heterozygous females [55;245;242;252-254].

Mutant Phenotypes

The most thoroughly characterized PLP mutation occurs in the jimpy mouse (PLP^{Jp}). An AG-to-GG transition in the 3′ acceptor splice site of intron 4 removes exon 5 during splicing (deleting 74 bp from the PLP/DM20 mRNAs) and produces a frameshift in the ORF after Tyr²⁰⁶. Therefore, jimpy mRNA encodes wtPLP/DM20 sequence up to Tyr²⁰⁶, but contains an altered 36 residue C-terminal sequence which is unusually rich in Cys [67,68]. Jimpy mice develop tremor, followed by convulsions and premature death [70]. There is a severe deficiency in mature OLs, accompanied by astrocytosis and increased proliferation of OL precursors [71-73;77;79;80]. Jimpy OLs develop normally before the premyelinating stage, but then arrest and die at the onset of myelination. The surviving OLs (˜10% of the normal number) myelinate <2% of the jimpy CNS axons, and the CNS myelin is either thin and poorly compacted, or displays an abnormal periodicity and lacks radial component. The jimpy mutation also represses transcription of myelin-specific genes, especially the PLP and myelin basic protein (MBP) genes. At the peak of myelination jimpy PLP/DM20 mRNAs are expressed at 5-10% of normal levels, and a PLP:DM20 ratio of 1 reflects the immature state of OL. The jimpy PLP/DM20 proteins are expressed at <0.5% of normal levels and cannot be detected in myelin. They appear to be retained in the endoplasmic reticulum (ER) and rapidly degraded [69;70;256].

In contrast, the rumpshaker mouse (Rsh, or PLP^{Jp-rsh}) which carries a single amino acid alteration in the PLP and DM20 proteins (I¹⁸⁶T) displays a considerably milder phenotype. These animals exhibit tremor and limb paresis, but reproduce effectively and have a near-normal lifespan. Although the Rsh brain displays significant hypomyelination, abnormal myelin periodicity and collapsed intraperiod lines, no significant oligodendrocyte loss is detected. In fact, oligodendrocyte number is slightly elevated, even though the cells appear to be developmentally delayed. This developmental delay is expressed as a low PLP-to-DM20 ratio, a ˜50% reduction in myelin gene expression and the detection of only the DM20 protein isoform in compact myelin [99;257-259]

In humans, mutations in the PLP gene are associated with two allelic diseases, Pelizaeus-Merzbacher Disease (PMD) and Spastic Paraplegia Type 2 (SPG2) [260;261.] PMD is characterized by severe CNS dysmyelination and loss of oligodendrocytes and can, be classified into three subtypes: connatal, transitional, and classic [262].

SPG2 patients have less dysmyelination than in PMD, and exhibit hyperreflexia, spastic gait, and some autonomic dysfunction. These patients can reproduce, have a normal lifespan, and show little impairment of speech and cognition. The SPG2 phenotype resembles mild PMD patients [263]. PLP gene duplications are the most common genetic abnormality associated with PMD, accounting for 60-70% of cases [57;58;264-267].

In-Vivo Experimental Systems.

The complexity of the PLP-associated disease phenotypes can be explained by three distinct mechanisms: (1) gene dosage effects, that is, hypo- or hyperactivity of the PLP gene products; (2) loss of function of the PLP gene products, or (3) gain of function (i.e., abnormal activity of the PLP gene products) [53]. Whereas the PLP null phenotype could be completely rescued by breeding with PLP/DM20 overexpressing strains supplying both proteolipid proteins, it was only partially rescued by supplying each protein isoform individually [61]. This contrasted with complementation studies in the jimpy (severe phenotype) and rumpshaker (mild phenotype) mutant mice using proteolipid overexpressing rodent strains. For the jimpy mouse cross, the “rescued” animals remained clinically indistinguishable from the nontransgenic jimpy mouse even though ultrastructural examination revealed that animals overexpressing both normal proteolipid protein isoforms showed increased numbers of myelinated axons and OL survival, coupled with improved myelin structure [103]. This inability to rescue the jimpy phenotype is attributed to a “dominant negative phenotype” associated with this mutation [102,103].

Similar attempts to rescue the rumpshaker phenotype by breeding PLP^{Jp-rsh} animals with a PLP protein overexpressing strain found that the “rescued” animals displayed the mutant phenotype and no obvious reduction in tremor severity. This occurred even though the transgenic myelin sheaths appeated structurally normal and stained strongly with PLP specific antibodies. Furthermore, PLP transgene expression did not significantly enhance myelination or OL maturation. Therefore, it has been suggested that the phenotypic severity associated with proteolipid gene mutations is defined by two functional activities: one function essential for glial cell survival during development (presumably regulated by DM20 expression) and another function essential for normal myelin compaction and stability in adulthood (presumably regulated by PLP expression) [54;259;268;269].

“Internal Ribosome Entry Site” (IRES)

The presence of low molecular weight (“LMW”) proteolipids in myelin [234;258;270-273] was thought to result from proteolysis of full length PLP/DM20 proteins. The present inventors' discoveries, disclosed herein, provide a better best explanation.

Translation initiation in eukaryotes mostly occurs via a “cap-dependent” scanning mechanism that involves the recognition m⁷G cap at the 5′end of the mRNA by initiation factor eIF4F which recruits the eIF2-GTP-Met-tRNA^{Met} and 40S ribosomal complexes to the mRNA. The resultant preinitiation complex scans the mRNA until it encounters an initiator AUG codon. An alternative, “cap-independent” mechanism of translation initiation involves ribosomal binding to a cis-acting mRNA element known as an internal ribosome entry site (IRES) which allows translation from internal AUG codons. This mechanism requires all of the canonical initiation factors (except for eIF4F) and one or more IRES trans-acting factors (ITAFs). ITAFs represent a diverse group of ssRNA binding proteins that act as RNA chaperones and thus facilitate IRES folding into an active conformation. It has been proposed that differences in temporal and spatial availability of ITAFs determine IRES activity [reviewed in 121;132;135;139].

According to the “first AUG rule,” 90-95% of all vertebrate mRNAs are translated starting at the first proximal AUG in a favorable sequence context. In higher eukaryotes, the consensus sequence GCCGCCA/GCCAUGG [SEQ ID NO:36] has been defined as the optimal translation initiation sequence. Although each of the nucleotides from position −1 through −6 (where the A of AUG is designated as position 1) are important for efficient start codon recognition, a purine in position −3 (usually A) is the most highly conserved nucleotide in eukaryotic mRNAs. If a purine nucleotide is present in position −3, sequence deviations can be tolerated in the remainder of the consensus sequence. However, in the absence of a purine in position −3, a G in position +4 becomes essential for efficient translation initiation.128 (part of the so-called “Kozak” rules).

The remaining 5-10% of vertebrate mRNAs do not follow the “first AUG rule” and, under restricted conditions, initiate from downstream AUG codons. If the distance between the 5′ end of mRNA and the first AUG codon is <10 nucleotides, ribosome formation often occurs on the second AUG codon [129] Similarly, an unfavorable sequence at the first AUG codon can promote ribosome read-through to downstream codons. This process is commonly termed “leaky scanning” translation [130]. Finally, when the first proximal AUG codon lies within a short ORF (often termed an upstream ORFs or uORFs), ribosomes can reinitiate at proximal start codons. In general, upstream AUG codons and short uORFs effectively limit downstream translation reinitiation [121,128,131].

IRES elements have been identified in a number of eukaryotic mRNAs [140], including genes that encode key regulatory proteins, such as translation initiation factors [152;186], transcription factors [145;187;190], oncogenes [187;274], kinases [142], growth factors [151;167;181;275], survival factors [176], and regulators of cell death [186;188]. IRES elements ensure the efficient expression of these proteins during nuclear inactivity or acute cellular stress when “cap-dependent” translation initiation is inhibited (i.e., apoptosis, starvation, γ-irradiation, hypoxia, mitosis, or terminal differentiation) [reviewed in 132;135;139;140;172;175;176].

The present inventors disclose herein and have unpublished evidence for two C-terminal LMW PLP peptides using their cell based ex-vivo system; production of these fragments could not be linked to any of the proteolytic systems tested. As described herein, a dramatic induction of the LMW proteolipid protein synthesis accompanies apoptotic cell death. Rather, the Examples below describes the production of these novel proteolipid proteins by internal translation initiation at an IRES.

Growth Factor Control of Oligodendrocyte Development.

The number of mature myelinating OLs is determined by the proliferative rate of OL progenitors (OLPs) and by elimination of the extra oligodendroglial lineage cells via the process of programmed cell death (i.e., apoptosis). Growth factors derived from the neighboring neuronal, astroglial, and oligodendroglial cells regulate both of these processes. For example, the proliferation and survival of OLPs (pre-progenitor and progenitor stages) is largely determined by the availability of PDGF and a series of synergistic trophic factors (i.e. inulin-like growth factor I (IGF-I), and members of the neuregulin family) secreted by astrocytes [86-89]. bFGF, NT3, ciliary neurotrophic factor (CNTF), and astrocyte derived chemokine CRO-1 stimulate PDGF receptor synthesis and produce a synergistic response with PDGF-A in OL precursors [90-94]. In contrast, premyelinating and myelinating OLs lose their dependence on astrocytic signals and seek survival factors associated with axons. In this manner, transforming growth factor-β (TGFβ) inhibits PDGF-A action and promotes differentiation of OLPs into pre-OLs [95]. At later stages of OL differentiation (when immature and mature OLs appear), IGF-I, NT3, LIF, CNTF, and GGF/NRG function as survival factors [88,92,93,96,97]. In the late stage myelinating cell, PDGF-A, GGF/NRG, thyroid hormone and cAMP signaling systems promote myelin-specific gene expression and induce myelination, while LIF exhibits an inhibitory effect^{97,98}. Therefore, the temporal stages of oligodendrocyte differentiation require distinct growth factor systems for commitment to terminal differentiation. Given the complexity of OL differentiation and the excessive number of currently defined growth factor responses, it seems likely that many undiscovered trophic factors regulate proliferation, migration, commitment and terminal differentiation of NS cells in the adult brain. The present invention is directed to PIRP growth factors and their activity tin the neural/glial differentiation process.

Revisiting the Function of PLP/DM20 Gene Products.

In general, the mutations associated with jimpy mice and myelin deficient (md) rats increased proliferation and reduced maturation of oligodendrocyte progenitors in-vivo, followed by the death of mature oligodendrocytes at the onset of myelination. Whereas jimpy and md OLs could reach the immature OL stage in culture, (, the mutant OLs failed to elaborate myelin-like membranous sheets and died via caspase-3 mediated apoptosis at an immature stage. The onset of cell death correlated with a strong induction of PLP protein expression that could not be prevented by treatment with CNTF, NT3, LIF, or bFGF [81;84;85;276;277]. Even though caspase-3 inhibitors promoted md rat oligodendrocyte survival ex-vivo, the “rescued” cells failed to differentiate and express mature myelin markers [277]. Moreover, survival of jimpy oligodendrocytes could be improved by the addition of medium conditioned by cells expressing wild type PLP/DM20 proteins. The “rescued” jimpy oligodendrocytes expressed higher amounts of myelin specific proteins (i.e., PLP and MBP) and elaborated myelin-like membranous sheets. Furthermore, cells expressing the PLP isoform had a greater effect on the numbers of surviving jimpy oligodendrocytes, while cells expressing the DM20 protein had a greater effect on the number and size of the membranous sheets [105]. These results suggest that the PLP/DM20 proteins promote myelination and regulate survival of immature and differentiated oligodendrocytes. It has been proposed that this activity is regulated by an undefined function of the PLPs. Preliminary studies using nonglial cells transduced to express the wild type PLPs described a secreted, soluble factor that stimulated proliferation of oligodendroglial and astroglial lineage cells. This activity could be mimicked by a peptide fragment corresponding to the residues 215-232 in the PLP protein sequence (180-197 in the DM20) [104;106]. This sequence is indicated below in the annotated PLP/DM20 sequence.

The specificity of this activity was defined by cells expressing mutant PLP/DM20 proteins (i.e., PLP^{Jp}, PLP^{Jp-msd}, and PLP^{Jp-rsh} variants) which failed to produce a similar proliferative effect. Inspection of these mutants suggested that a C-terminal proteolipid protein sequence was involved in OL-neuronal signaling, and the absence of this signal contributed to the axonal abnormalities of PLP^{Jp} and plp^{null} mice [107]. Indeed, the present inventors have shown that the PLP/DM20 gene products exert a direct effect on neuronal survival, and that PLP isoform overexpression is associated with decreased neuronal viability [108].

Role of Cell Death in Oligodendrocyte Development.

Programmed cell death is a normal feature of OL development that adjusts the number of myelinating cells to the number of axons. Studies in rats show that as many as 50% of the newly formed OLs in the optic nerve die within 2-3 days after they are generated [204-207]. Survival of OLPs is largely determined by the availability of PDGF-A and the synergistic trophic factors (see above) secreted by astrocytes [278;279]. In contrast, premyelinating and myelinating oligodendrocytes lose their dependence of the astrocytic signals and begin to derive their survival factors from axons. Any cell that fails to establish axonal contact is eliminated through the lack of trophic support, so that the number of myelinating oligodendrocytes always equals the number of axons to be myelinated [280;281].

In certain cases, developmental cell death occurs independently of the trophic factor availability. Programmed cell death can be initiated when a cell receives opposing signals regulating proliferation and growth arrest. Thus, the oligodendrocyte progenitors expressing the proliferative cell cycle molecules are eliminated in response to differentiative signals, while mature oligodendrocytes undergo apoptosis in upon exposure to strong mitogens [282]

PLP/DM20 gene products could regulate OL death and survival in several ways. First, a secreted C-terminal peptide might have a direct effect on oligodendrocyte survival at different developmental stages. Spassky et al. [283;284] defined two distinct lineages of OLPs. The first lineage expresses the PDGFα receptor and depends upon astrocytic PDGF-A for proliferation and survival. In contrast, the second lineage is defined by early expression of the DM20 mRNA and a lack of PDGFα expression. Since the second lineage does not depend upon PDGF-A for growth and survival, these functions may be regulated by a DM20 derived trophic factor. This factor could enhance survival of the PDGF-A dependent lineage by increasing the number of astrocytes [283;284, 106] ). At a later stage of development, the PLP/DM20 proteins may contribute to OL survival by mediating OL-neuronal cell communications Indeed, studies with compound heterozygous animals showed that wild type oligodendroglial cells were much more likely to establish axonal contact and survive than PLP deficient or mutant oligodendrocytes [285;254].

Recapitulation of Developmental Program in Demyelinating Disease.

Demyelination in MS and its animal model, EAE, is associated with loss of OLs by both apoptotic and necrotic mechanisms [286;287;282]. Dowling et al. [287] showed that 14-40% of all dying cells within acute and chronic MS lesions are of oligodendroglial lineage. However, the same authors detected a remarkable amount of cell proliferation coexisting in the same white matter areas where oligodendroglial death was observed, reflecting the attempts at remyelination and lesion repair.

Remyelination may involve full or partial recapitulation of molecular events occurring during OL development and myelinogenesis. Consistently, the onset of remyelination in MS and EAE is accompanied by increased expression of developmentally specific isoforms of PLP (i.e., the DM20 protein) and MBP (i.e., the exon 2 containing 20.2 and 21.5 kDa proteins) [109, 110]. Moreover, the extent of spontaneous recovery from EAE correlates directly with increased expression of the DM20 isoform, regardless of whether the remission occurs after EAE onset or relapse. The reinduction of DM20 expression appears to be specific for the active phase of sustained remission, since the level of DM20 returns to normal during the long-term remission phase [109].

### SUMMARY OF THE INVENTION

In their previous efforts to define proteolipid protein turnover via the ubiquitin/proteasome system, the present inventors produced a series of stably transfected cell lines that expressed various wild type (wt) and mutant PLP/DM20 proteins (primarily as readily detectable fluorescent fusion proteins). In addition to the expected recombinant full length polypeptide, Western blot analysis consistently revealed the presence of low molecular weight (LMW) PLPs, the expression of which dramatically increased in response to cellular stress and apoptotic cell death. Immunochemnical detection indicated that the LMW polypeptides were derived from C-terminal sequences of PLP/DM20. However, repeated efforts could not link their biosynthesis to any proteolytic system. Since cellular stress/death affects cap-dependent translation, the present inventors conceived, and went on to prove, that the LMW polypeptides, now known as PIRP-M and PIRP-L, are produced by alternative translation of the PLP/DM20 mRNAs from internal AUG codons. Using site directed mutagenesis of potential translation initiation codons and deletion of the vector promoter sequences, a functional IRES element was identified in the proteolipid gene sequence which produced two LMW polypeptides (a 7 kDa polypeptide termed the PIRP-L protein and 10 kDa polypeptide named PIRP-M). The discovery of the PLP IRES represents the fifth major gene expressed in OLs to include IRES sequences. It is important to note that one of the IRES-specific proteins encompasses the PLP/DM20 growth factor sequence whilch had tentatively been assigned to the C-terminus in earlier studies.

Thus, according to the present invention, the 7 kDa polypeptide (SEQ ID NO:8) named PIRP-L and the 10 kDa polypeptide (SEQ ID NO: 6) named PIRP-M are OL growth factors. As conceived by the present inventors, and supported by the regulated biosynthesis and sequence of the PLP IRES proteins, the PIRPs are part of an important regulatory system that govern OL responses during development, stress and remyelination. Novel, alternative biological activities that are characteristic of the transcripts and/or the proteins encoded by normal and mutant PLP/DM20 genes are not necessarily related to the function of the previously described and well-known full-length PLP/DM20 proteins.

Throughout this document, mutations (resulting in amino acid substitutions) are indicated by a single amino acid code letter representing the wt residue, followed by a number, indicating sequence position in the PLP protein, followed by the letter representing the mutant (substituted) residue at that position. If more than one mutation was present, it is set off by a slash (“/”).

According to the present invention, translation of the PIRPs during apoptosisresults in production of trophic factors which are released by dying OLs that recruit and promote the survival of remyelinating cells. A similar relationship between dying and surviving cells of OL lineage are predicted to exist during development.

In addition to its potential protective role in demyelinating disease, prolonged secretion of the PLP/DM20 derived growth factor, and specifically, PIRP-M (SEQ ID NO:6) is associated with increased risk of two types of brain tumors, oligodendrogliomas and astrocytomas. Indeed, a subpopulation of patients with relatively mild MS has been reported to develop gliomas 8-15 years after the initial diagnosis [111-119]. According to the present invention, an association between a mild course of MS and neoplasia is rooted in high levels of remyelination and growth factor secretion that triggers and drives neoplastic transformation of glial stem cells. Alternatively, enhanced growth factor secretion by tumor cells could positively effect myelin repair and contribute to a mild MS phenotype.

Specifically, the present invention is directed to an isolated, recombinant t polypeptide molecule comprising a first amino acid sequence which is a fragment of a native proteolipid protein (preferably mammalian or human PLP/DM20) having a wild type or mutant sequence as compared with the native sequence of said proteolipid protein, and optionally comprising a second amino acid sequence fused in frame thereto to create a fusion polypeptide, which first polypeptide is encoded by an mRNA having an Internal Ribosome Entry-Site ((IRES) wherein translation of the mRNA initiates at said IRES, such that the N-terminal amino acid residue of said first polypeptide corresponds to an internal residue of said proteolipid protein.

The first polypeptide above may be
(a) PIRP-M, having the amino acid sequence SEQ ID NO:6;
(b) PIRP-L, having the amino acid sequence SEQ ID NO:8;
(c) a fusion polypeptide of (a) or (b) wherein said second amino acid sequence encodes a naturally fluorescent protein or peptide, preferably yellow or green green fluorescent protein (GFP) or a fluorescent homologue thereof;
(d) a His-tagged fusion polypeptide of PIRP-M having the amino acid sequence SEQ ID NO:12;
(e) a His-tagged fusion polypeptide of PIRP-L having the amino acid sequence SEQ ID NO:16; or
(f) PIRP-J having a mutant sequence compared to said proteolipid protein, the sequence of said PIRP-J being SEQ ID NO:18, or a human homologue thereof.

Also provided is an isolated nucleic acid encoding any of the above polypeptides, the mutant sequences thereof, or fusion polypeptides thereof. The nucleic acid may be a DNA molecule or an RNA molecule.

A preferred nucleic acids encodes
PIRP-M and has a nucleotide sequence SEQ ID NO:5 or SEQ ID NO:9;
PIRP-L and has a nucleotide sequence SEQ ID NO:7 or SEQ ID NO:13;
PIRP-J and has a nucleotide sequence SEQ ID NO:17.
His-tagged fusion polypeptide of PIRP-M and has a nucleotide sequence SEQ ID NO:11;
His-tagged fusion polypeptide of PIRP-L and has a nucleotide sequence SEQ ID NO:15;

The above nucleic acid may be operatively linked to a promoter, preferably one which is expressed in a mammalian cell such as a neuronal cell, a glial cell or a stem cell. A preferred glial cell is an oligodendrocyte. A preferred stem cells are neural stem cell, an oligodendrocyte progenitor cell, an embryonic stem cell or a hemopoietic stem cell.

Also provided is a vector comprising the above nucleic acid. Preferred vectors include PLP-GFP/DM20-GFP; PLP-GFP/DM20-GFP Tet-On; PLP-GFP/DM20-GFP M1L; PLP-GFP/DM20-GFP M1L/M205L; PLP-GFP/DM20-GFP M1L/M234L; PLP-GFP/DM20-GFP M1L/M205L/M234L; PLP-GFP/DM20-GFP Pro-; JPLP-GFP/JDM20-GFP; JPLP-GFP/JDM20-GFP M1L; JPLP-GFP/JDM20-GFP M1L/M205L; RshPLP-GFP/RshDM20-GFP M1L; PLP-GFP/DM20-GFP M1L/K268R; PLP-GFP/DM20-GFP M1L/K275R; PLP-GFP/DM20-GFP M1L/K268R/K275R; and PLP-GFP/DM20-GFP M1L/R272K.

One embodiment is directed to a an expression vector or cassette comprising the above nucleic acid operatively linked to
(a) a promoter; and
(b) optionally, additional regulatory sequences that regulate expression of said nucleic acid in a eukaryotic cell.

The expression vector or cassette preferably comprises a vector selected from the group consisting of pCMV; pEGFP-N1; pEYFP-N1; pEGFP-Tet-On; pBluescript II KS+; and pET-14b. Preferred expression vectors or cassettes include 205M-CMV/234M-CMV; 205M-His-CMV/234M-His-CMV; 205M-BsKS+/234M-BsKS+; 205M-His-BsKS+/ 234M-His-BsKS+; and 205M-ET-14b/234M-ET 14Also included is a cell which has been modified to comprise the above nucleic acid, vector, preferably expression vector and preferably to express the polypeptide. Preferably, the cell is a mammalian cell, more preferably a human cell. The cell types noted above are preferred

Also provided are pharmaceutical compositions comprising pharmaceutically acceptable excipient in combination with any of the above polypeptides, nucleic acids, expression vectors or cells, as may be used to treat a disease or condition treatable by administration of, or expression of the various PIRP molecules described herein.;

Also provided are treatment methods to treat such conditions, or to stimulate neural stem cell differentiation in a subject or to promote neural cell survival, or, when appropriate, differentiation and or proliferation. Preferred expression vectors are translated during apoptosis of neural cells or OL leading to positive regulation of downstream sequences, for example after binding apoptosis-derived ITAF proteins which bind to the “Exon 4-requlatory sequences or the PLP/DM20 gene.

### BRIEF DESCRIPTION OF THE DRAWINGS

FIGS. 1A-1C show the proposed structure of plasma membrane-bound PLP protein and exemplify proteolipid protein expression in mammalian cells. FIG. 1A is a schematic shows is a tetraspan membrane structure with two C-terminal ubiquitination sites (Ub), two extracellular disulfide bonds and six fatty acylated intracellular cysteines. 293 cells expressing the fluorescent PLP-GFP fusion protein are shown in FIG. 1B. 293 cells expressing DM20-GFP fusion protein is shown in FIG. 1C. Both recombinant proteins localize to a perinuclear Golgi structure (solid arrow) and plasma membrane microvilli (hatched arrows).

FIGS. 2A-2D characterizes internal translation initiation events. FIG. 2A is schematic showing PLP/DM20-GFP mRNAs with all potential in-frame (top arrows) and out-of-frame (bottom arrows) initiation codons. Ribosome scanning from AUG 1 to AUG 205/234 would be blocked by these out of frame codons. The AUG “cluster” surrounding the AUG 205 codon is noted. Lines below the mRNA diagram represent a series of deletion clones designed to map the proteolipid IRES element. FIG. 2B shows schematics of the Met¹Leu (M1L) and CMV promoter deletion (Pro⁻) vectors. Internal proteins were synthesized from the M205 and M234 codons in cells expressing the MIL mutant RNA. FIGS. 2C1, 2C2-2D show gel patterns of Western blot analyses detecting low molecular weight (LMW) protein expression. In FIG. 2C1, expression of LMW proteins (<40 kDa) is induced in PLP/DM20-GFP-expressing cells by MG132 treatment. In FIG. 2D, MG132-induced apoptosis produces the same LMW proteins in PLP/DM20-GFP and M1L PLP/DM20-GFP expressing cells. FIG. 2C2 shows exclusion of an internal cryptic promoter activity from the PLP and DM20 cDNAs by deletion of the CMV promoter.

FIGS. 3A-3C: Mutagenesis localizes the IRES and internal translation initiation sites in the PLP and DM20 mRNA sequences. FIG. 3A is a set of schematics showing Met¹ (M1L), Met²⁰⁵ (M205L) and Met²³⁴ (M234L) mutant transcripts. FIGS. 3B-3C are gel patterns showing Western blot analysis of the Proteolipid IRES Proteins (PIRPs) expressed from the Met mutant templates. PIRP-M (˜38 kDa) is translated from M205, whereas PIRP-L (˜34 kDa) is synthesized from M234.

FIGS. 4A-4C: Translation of PIRP proteins from jimpy (jp) and rumpshaker (rsh) transcripts. FIG. 4A is a set of schematics of vectors designed to detect PIRP protein expression from the jp and rsh transcripts. The jp mutation results in the deletion of exon 5 and a C-terminal frameshift which alters the M205 PIRP sequence (this frameshifted protein was termed “PIRP-J”). The rsh mutation converts Ile186 to Thr. To verify that a jp-specific PIRP protein is derived by M205 initiation, the M205L mutation was introduced into the jp M1L transcripts. FIGS. 4B and 4C are gel patterns showing Western blot analysis of cell lines expressing the jp and rsh transcripts. FIG. 4B shows that a M205L mutation eliminates translation of the PIRP-J protein. FIG. 4ECshows that the rsh mutation does not affect PIRP-M and PIRP-L protein synthesis.

FIGS. 5A-5C are directed to native and 6×His-tagged PIRP proteins and the detection of secreted proteins in serum free media. FIG. 5A shows schematics of monocistronic PIRP-M expressed from the pCMV vector (top) and the 6×His-tagged derivative (bottom). FIG. 5B shows schematics of monocistronic PIRP-L expressed from the pCMV vector (top) and a 6×His-tagged derivative (bottom). FIG. 5C is a gel pattern demonstrating detection of LMW proteins secreted by PIRP-L expressing cells. Confluent cultures were maintained for >48 hrs prior to collection of conditioned media (CM). CM (50 ml) was concentrated using CentriPrep-10 (general concentration and desalting step) and CentriCon-30 filtration. Protein concentrates were resolved on 20% SDS-PAGE gels and secreted proteins detected by silver staining. PIRP-L cell lines secreted a 7 kDa protein which is unique to these cells. A related protein was detected in PIRP-M cells (not shown).

FIGS. 6A-6C are bar graphs showing growth factor activity associated with the PIRP-M and PIRP-L proteins. FIG. 6A shows a positive effect of PIRP protein expression on cellular transformation. Colony number was determined after calcium phosphate transfection of 293 cells and G418 selection. The control plasmid was pBSII vector comprising the PIRP-M gene but no mammalian selectable marker. The PLP/DM20-GFP Pro-constructs are missing the CMV promoter and the PLP/DM20-GFP M1L vectors contain the Met¹ mutation and are unable to translate the full-length proteolipid proteins (see description of FIGS. 2B-2C). The PIRP expression vectors are shown in FIGS. 5A and 5B. Increase in colony number following PIRP expression was highly stastically significant (Student's t-test). These results were replicated with independent DNA preparations. FIG. 6B shows the anti-apoptotic phenotype associated with PIRP protein expression. Pools of 293 cells derived from the transfectants (see above) were grown in toxic (50 μM) and subtoxic (25 μM) concentrations of MG132 for 24 hrs. Viable cells were identified using Trypan Blue exclusion. The graph shows the % of control cell number (taken as 100%). PIRP-M expressing cells exhibited increased cell number in untreated and treated samples. The PIRP-L and PIRP-M/PIRP-L expressing cell lines displayed a similar trend but the absolute increase in cell number was lower. These results did not reach statistical significance (two replications, n=2). FIG. 6C shows proliferation and viability of 293 cells grown in PIRP CM prepared from PIRP expressing and 293 control cell lines. Subconfluent 293 cultures were treated with CM for 48 hrs and 72 hrs. Total and viable cell numbers were determined using Trypan Blue exclusion and expressed as % of control (293 cells treated with 293 CM) cell number. Consistent with the results in FIG. 6B, the PIRP-M CM induced a statistically significant increase in cell number at both timepoints. A similar trend was observed with the PIRP-L and PIRP-M/PIRP-L CMs, but with greater variability in responses. These results indicate that the PIRP proteins are growth factors which are either secreted or induce the secretion of factors which increase cellular viability and anti-apoptotic activity.

FIGS. 7A and 7B show two models for internal start codon selection in the PLP/DM20 mRNAs. In Model 1 (FIG. 7A), a single ribosome binding site is located upstream of M205 and used during steady state and stress conditions. Under steady state conditions, the ribosome does not initiate translation from M205 but scans to M234. In contrast, a stress response induces binding of stress-specific IRES trans-acting factors (ITAFs), a conformational change in the IRES structure, and preferential translation from M205. Model 2 (FIG. 7B assumes that two distinct ribosome binding sites (RBSs) are located upstream of M205 in Exon 4 and of M234 in Exon 5. During steady state conditions, the Exon 4 RBS is inactive and ribosomes bind to Exon 5. During stress, ITAF protein binding changes the conformation of Exon 4 and allows ribosomes to access. The model accounts for the possibility that Exon 4 and Exon 5 simultaneously bind ribosomes on the same mRNA (top right), or whether ribosome binding to Exon 4 prevents translation from Exon 5 (bottom right).

FIG. 8 is a diagram describing analysis of the effect of the PIRP proteins on the 4−/4+ Embryonic Stem (ES) Cell in vitro differentiation protocol. The 4−/4+ retinoic acid protocol for differentiating ES cells into neurons was developed by others to produce ologodendrocyte-enriched cultures. The first study (“black box” on the right) tests the PIRP proteins for their alteration of D3 ES cell differentiation. Purified PIRP-M and PIRP-L proteins are added (or transgenes are expressed) at defined steps of the differentiation process to determine whether these proteins augment, replace or inhibit the activity of growth factors used for such differentiation in vitro. During the initial retinoic acid (RA) treatment, the PIRP proteins might augment effects of RA (increasing its potency), replace RA (induce differentiation in its absence) or inhibit RA action. The role of PIRP proteins as the secreted factors in “oligosphere” (OS) conditioned media needed in the last step of the procedure are also tested. A second analysis (“black box” on left) test whether the jp PIRP protein (PIRP-J) is the dominant negative factor responsible developmental defects in jp animals. Addition of the purified PIRP-J protein or expression of the PIRP-J transgene during D3 differentiation should may reduce cell viability and lead to stage-specific cell death.

### DESCRIPTION OF THE PREFERRED EMBODIMENTS

The present inventors have discovered novel products of the PLP/DM20 gene.

The nucleotide sequence encoding human PLP (SEQ ID NO:1 and the full length protein SEQ ID NO:2, are shown below. The stop codon, TGA, is shown. Human genomic DNA includes 3′ untranslated segment, the first 16 nucleotides of which are TACACTGGTITCCCTG. Numbering of nucleotides is above, and of amino acid residues is below, the relevant sequences. Annotations are explained following the full length sequence.

```
FULL LENGTH PLP SEQUENCE (ANNOTATED*)
                                  15 30 45
ATG GGC TTG TTA GAG TGC TGT GCA AGA TGT CTG GTA GGG GCC CCC TTT GCT TCC
Met Gly Leu Leu Glu Cys Cys Ala Arg Cys Leu Val Gly Ala Pro Phe Ala Ser
(0) 5 10 15
                                                                                                                        >>>>>>>>>>>
          60 75 90 105
CTG GTG GCC ACT GGA TTG TGT TTC TTT GGG GTG GCA CTG TTC TGT GGC TGT GGA
Leu Val Ala Thr Gly Leu Cys Phe Phe Gly Val Ala Leu Phe Cys Gly Cys Gly
                  20 25 30 35
>>>>>>>>>>>>>>>>>>>>>>>>>>TMD-1 >>>>>>>>>>>>>>>>>>>>>>>>>>>>>>>>>>>>>>
                        120 135 150
CAT GAA GCC CTC ACT GGC ACA GAA AAG CTA ATT GAG ACC TAT TTC TCC AAA AAC
His Glu Ala Leu Thr Gly Thr Glu Lys Leu Ile Glu Thr Tyr Phe Ser Lys Asn
                                  40 45 50
165 180 195 210
TAC CAA GAC TAT GAG TAT CTC ATC AAT GTG ATC CAT GCC TTC CAG TAT GTC ATC
Tyr Gln Asp Tyr Glu Tyr Leu Ile Asn Val Ile His Ala Phe Gln Tyr Val Ile
          55 60 65 70
                                        >>>>>>>>>>>>>>>>>>>>>>>>>>>>>>>>>>>>TMD-2 >>>>>>>>
                225 240 255 270
TAT GGA ACT GCC TCT TTC TTC TTC CTT TAT GGG GCC CTC CTG CTG GCT GAG GGC
Tyr Gly Thr Ala Ser Phe Phe Phe Leu Tyr Gly Ala Leu Leu Leu Ala Glu Gly
                          75 80 85
>>>>>>>>>>>>>>>>>>>>>>>>>>>>>>>>>>>>>>>>>>>>>>>>>>>>>>>>>>>>>>>>>
                                285 300 315
TTC TAC ACC ACC GGC GCA GTC AGG CAG ATC TTT GGC GAC TAC AAG ACC ACC ATC
Phe Tyr Thr Thr Gly Ala Val Arg Gln Ile Phe Gly Asp Tyr Lys Thr Thr Ile
  90 95 100 105
        330 345 360 375
TGC GGC AAG GGC CTG AGC GCA ACG GTA ACA GGG GGC CAG AAG GGG AGG GGT TCC
Cys Gly Lys Gly Leu Ser Ala Thr Val Thr Gly Gly Gln Lys Gly Arg Gly Ser
                110 115 120 125
                                                                ***************************************
                        390 405 420
AGA GGC CAA CAT CAA GCT CAT TCT TTG GAG CGG GTG TGT CAT TGT TTG GGA AAA
Arg Gly Gln His Gln Ala His Ser Leu Glu Arg Val Cys His Cys Leu Gly Lys
                                130 135 140
*******************************PLP-specific sequence****************
435 450 465 480
TGG CTA GGA CAT CCC GAC AAG TTT GTG GGC ATC ACC TAT GCC CTG ACC GTT GTG
Trp Leu Gly His Pro Asp Lys Phe Val Gly Ile Thr Tyr Ala Leu Thr Val Val
        145 150 155 160
*************************** >>>>>>>>>>>>>>>>>>>>>>>>>>>>>>>>>>>>>>>>>>>
                495 510 525 540
TGG CTC CTG GTG TTT GCC TGC TCT GCT GTG CCC GTG TAC ATT TAC TTC AAC ACC
Trp Leu Leu Val Phe Ala Cys Ser Ala Val Pro Val Tyr Ile Tyr Phe Asn Thr
                        165 170 175
>>>TMD-3 >>>>>>>>>>>>>>>>>>>>>>>>>>>>>>>>>>>>>>>>>>>>>>>>>>>>
                                555 570 585
TGG ACC ACC TGC GAC TCT ATT GCC TTC CCC AGC AAG ACC TCT GCC AGT ATA GGC
Trp Thr Thr Cys Asp Ser Ile Ala Phe Pro Ser Lys Thr Ser Ala Ser Ile Gly
180 185 190 195
        600 615 630 645
AGT CTC TGT GCT GAC GCC AGA ATG TAT GGT GTT CTC CCA TGG AAT GCT TTC CCT
Ser Leu Cys Ala Asp Ala Arg Met Tyr Gly Val Leu Pro Trp Asn Ala Phe Pro
                200 205 210 215
                        660 675 690
GGC AAG GTT TGT GGC TCC AAC CTT CTG TCC ATC TGC AAA ACA GCT GAG TTC CAA
Gly Lys Val Cys Gly Ser Asn Leu Leu Ser Ile Cys Lys Thr Ala Glu Phe Gln
                                220 225 230
705 720 735 750
ATG ACC TTC CAC CTG TTT ATT GCT GCA TTT GTG GGG GCT GCA GCT ACA CTG GTT
Met Thr Phe His Leu Phe Ile Ala Ala Phe Val Gly Ala Ala Ala Thr Leu Val
        235 240 245 250
                                >>>>>>>>>>>>>>>>>>>>>>>>>>>>>>>TMD-4 >>>>>>>>>>>>>>>>>
                765 780 795 810
TCC CTG CTC ACC TTC ATG ATT GCT GCC ACT TAC AAC TTT GCC GTC CTT AAA CTC
Ser Leu Leu Thr Phe Met Ile Ala Ala Thr Tyr Asn Phe Ala Val Leu Lys Leu
                        255 260 265
>>>>>>>>>>>>>>>>>>>>>>>>>>>>>>>>>>>>>>>>>>>>>>>>>>>>>>>>>>>>>>>>
                                825
ATG GGC CGA GGC ACC AAG TTC TGA TACACTGGTTTCCCTG
Met Gly Arg Gly Thr Lys Phe
270 275
```

Comments on Annotations

TMD-1, 2, 3 and 4: Four transmembrane domains (TMD1-TMD4) are indicated by >>>>>. A PLP-specific sequence indicated by ***** (35 aa's absent from DM20).

Underscored amino acid residues 215-232 in the PLP protein sequence (180-197 in the DM20) are described by Yamada et al., 1999) as being important to growth factor activity.

Exon 4 at the nucleotides TTT (at nt456-458) and continues through nt 622, the first G in the codon for Gly²⁰⁷ . Thus Exon 4 corresponds to nt 456-622 of SEQ ID NO:1. As will be discussed below, Exon 4 serves as a regulatory sequence that can help drive translation of an immediately downstream sequence after Exon 4 activation with, for example, and ITAF-1 protein is produced as part of an apoptotic response.

The polypeptide “fragment” of the sequences provided above, corresponding to human DM20 (nucleotide sequence is SEQ ID NO:3 and amino acid sequence is SEQ ID NO:4) is given below. It differs from the full length PLP sequence by removal of the segment designated “PLP-specific sequence” above The numbering is adjusted accordingly.

```
Annotated DM20 Sequence (annotations as above)
                                15 30 45
ATG GGC TTG TTA GAG TGC TGT GCA AGA TGT CTG GTA GGG GCC CCC TTT GCT TCC
Met Gly Leu Leu Glu Cys Cys Ala Arg Cys Leu Val Gly Ala Pro Phe Ala Ser
(0) 5 10 15
                                                                                                                        >>>>>>>>>>>
        60 75 90 105
CTG GTG GCC ACT GGA TTG TGT TTC TTT GGG GTG GCA CTG TTC TGT GGC TGT GGA
Leu Val Ala Thr Gly Leu Cys Phe Phe Gly Val Ala Leu Phe Cys Gly Cys Gly
                  20 25 30 35
>>>>>>>>>>>>>>>>>>>>>>>>>>TMD-1 >>>>>>>>>>>>>>>>>>>>>>>>>>>>>>>>>>>>>>
                        120 135 150
CAT GAA GCC CTC ACT GGC ACA GAA AAG CTA ATT GAG ACC TAT TTC TCC AAA AAC
His Glu Ala Leu Thr Gly Thr Glu Lys Leu Ile Glu Thr Tyr Phe Ser Lys Asn
                                  40 45 50
165 180 195 210
TAC CAA GAC TAT GAG TAT CTC ATC AAT GTG ATC CAT GCC TTC CAG TAT GTC ATC
Tyr Gln Asp Tyr Glu Tyr Leu Ile Asn Val Ile His Ala Phe Gln Tyr Val Ile
          55 60 65 70
                                        >>>>>>>>>>>>>>>>>>>>>>>>>>>>>>>>>>>>TMD-2 >>>>>>>>
                225 240 255 270
TAT GGA ACT GCC TCT TTC TTC TTC CTT TAT GGG GCC CTC CTG CTG GCT GAG GGC
Tyr Gly Thr Ala Ser Phe Phe Phe Leu Tyr Gly Ala Leu Leu Leu Ala Glu Gly
                          75 80 85
>>>>>>>>>>>>>>>>>>>>>>>>>>>>>>>>>>>>>>>>>>>>>>>>>>>>>>>>>>>>>>>>>
                                285 300 315
TTC TAC ACC ACC GGC GCA GTC AGG CAG ATC TTT GGC GAC TAC AAG ACC ACC ATC
Phe Tyr Thr Thr Gly Ala Val Arg Gln Ile Phe Gly Asp Tyr Lys Thr Thr Ile
  90 95 100 105
        330 345 360 375
TGC GGC AAG GGC CTG AGC GCA ACG TTT GTG GGC ATC ACC TAT GCC CTG ACC GTT
Cys Gly Lys Gly Leu Ser Ala Thr Phe Val Gly Ile Thr Tyr Ala Leu Thr Val
                110 115 120 125
                                                                >>>>>>>>>>>>>>>>>>>>>>>>>>>>>>>>>>>>>>>
                        390 405 420
GTG TGG CTC CTG GTG TTT GCC TGC TCT GCT GTG CCC GTG TAC ATT TAC TTC AAC
Val Trp Leu Leu Val Phe Ala Cys Ser Ala Val Pro Val Tyr Ile Tyr Phe Asn
                                130 135 140
>>>>>>TMD-3 >>>>>>>>>>>>>>>>>>>>>>>>>>>>>>>>>>>>>>>>>>>>>>>>>>>>>>
435 450 465 480
ACC TGG ACC ACC TGC GAC TCT ATT GCC TTC CCC AGC AAG ACC TCT GCC AGT ATA
Thr Trp Thr Thr Cys Asp Ser Ile Ala Phe Pro Ser Lys Thr Ser Ala Ser Ile
                145 150 155 160
                495 510 525 540
GGC AGT CTC TGT GCT GAC GCC AGA ATG TAT GGT GTT CTC CCA TGG AAT GCT TTC
Gly Ser Leu Cys Ala Asp Ala Arg Met Tyr Gly Val Leu Pro Trp Asn Ala Phe
                        165 170 175
                                555 570 585
CCT GGC AAG GTT TGT GGC TCC AAC CTT CTG TCC ATC TGC AAA ACA GCT GAG TTC
Pro Gly Lys Val Cys Gly Ser Asn Leu Leu Ser Ile Cys Lys Thr Ala Glu Phe
180 185 190 195
        600 615 630 645
CAA ATG ACC TTC CAC CTG TTT ATT GCT GCA TTT GTG GGG GCT GCA GCT ACA CTG
Gln Met Thr Phe His Leu Phe Ile Ala Ala Phe Val Gly Ala Ala Ala Thr Leu
                200 205 210 215
                                        >>>>>>>>>>>>>>>>>>>>>>>>>>>>>>>TMD-4 >>>>>>>>>>>>>
                        660 675 690
GTT TCC CTG CTC ACC TTC ATG ATT GCT GCC ACT TAC AAC TTT GCC GTC CTT AAA
Val Ser Leu Leu Thr Phe Met Ile Ala Ala Thr Tyr Asn Phe Ala Val Leu Lys
                                220 225 230
>>>>>>>>>>>>>>>>>>>>>>>>>>>>>>>>>>>>>>>>>>>>>>>>>>>>>>>>>>>>>>>>>>>>
705 720
CTC ATG GGC CGA GGC ACC AAG TTC TGA
Leu Met Gly Arg Gly Thr Lys Phe
        235 240
```

As described in detail in the Examples, the present inventors have discovered two additional products produced from PLP/DM20 transcripts as a result of initiation from internal sites under conditions of stress and apoptosis. These fragments have functions and utilities that are described below.

One polypeptide fragment of with a molecular mass of about 10 kDa is synthesized upon initiation of translation from the Met 205-encoding ATG codon (AUG at the RNA level) when cap-mediated translation from Met¹ is inhibited in wt cells (or in the case of mutations that have eliminated the Met¹ codon). This polypeptide is interchangeably designated PIRP-M, the 10 kDa fragment, peptide, polypeptide or protein, as well as the Met²⁰⁵ protein. Its coding sequence, SEQ ID NO:5, is a fragment of SEQ ID NO:1 and its amino acid sequence, SEQ ID NO:6, is a fragment of SEQ ID NO:2. The numbering of the nucleotides retains the numbers of the full length coding PLP ORF.

```
PIRP M: 10 kDa PLP fragment (encoded by nt's 616-831 of SEQ ID NO:1)
                                630 645 660
ATG TAT GGT GTT CTC CCA TGG AAT GCT TTC CCT GGC AAG GTT TGT GGC TCC AAC
Met Tyr Gly Val Leu Pro Trp Asn Ala Phe Pro Gly Lys Val Cys Gly Ser Asn
                                  5 10 15
        675 690 705 720
CTT CTG TCC ATC TGC AAA ACA GCT GAG TTC CAA ATG ACC TTC CAC CTG TTT ATT
Leu Leu Ser Ile Cys Lys Thr Ala Glu Phe Gln Met Thr Phe His Leu Phe Ile
          20 25 30 35
                      735 750 765
GCT GCA TTT GTG GGG GCT GCA GCT ACA CTG GTT TCC CTG CTC ACC TTC ATG ATT
Ala Ala Phe Val Gly Ala Ala Ala Thr Leu Val Ser Leu Leu Thr Phe Met Ile
                          40 45 50
780 795 810 825
GCT GCC ACT TAC AAC TTT GCC GTC CTT AAA CTC ATG GGC CGA GGC ACC AAG TTC
Ala Ala Thr Tyr Asn Phe Ala Val Leu Lys Leu Met Gly Arg Gly Thr Lys Phe
55 60 65 70 (72)
```

Yet another internally initiated ˜7 kDa polypeptide “fragment is synthesized upon initiation of translation from the Met²³⁴-encoding ATG codon (AUG at the RNA level) when cap-mediated translation from Met¹ is inhibited in wt cells (or in the case of mutations that have eliminated the Met¹ and Met²⁰⁵ codons). This polypeptide is interchangeably designated PIRP-M, the PIRP-L fragment, peptide, polypeptide or protein, as well as the Met 234 protein. Its coding sequence, SEQ ID NO:7, is a fragment of both SEQ ID NO:1 and SEQ ID NO:5. Its amino acid sequence, SEQ ID NO:8, is a fragment of both SEQ ID NO:2 and SEQ ID NO:6. The numbering of the nucleotides retains the numbers of the full length coding PLP ORF.

```
PIRP L 7 kDa PLP Fragment (encoded by nt's 705-831 of SEQ ID NO:1)
705 720 735 750
ATG ACC TTC CAC CTG TTT ATT GCT GCA TTT GTG GGG GCT GCA GCT ACA CTG GTT
Met Thr Phe His Leu Phe Ile Ala Ala Phe Val Gly Ala Ala Ala Thr Leu Val
                                  5 10 15
                765 780 795 810
TCC CTG CTC ACC TTC ATG ATT GCT GCC ACT TAC AAC TTT GCC GTC CTT AAA CTC
Ser Leu Leu Thr Phe Met Ile Ala Ala Thr Tyr Asn Phe Ala Val Leu Lys Leu
          20 25 30 35
                                825
ATG GGC CGA GGC ACC AAG TTC
Met Gly Arg Gly Thr Lys Phe
                          40 (43)
```

The insert that is used for cloning the native PIRP-M or PIRP-L sequence or “optimized” or modified PIRP-M or PIRP-L sequence (shown below) has additional 5′ and 3′ nucleotides that introduce Kozak sequences to optimize these sequences for eukaryotic translation. Such sequences are discussed above in the Background Section. These untranslated sequences are also used in the His-tagged analogues of PIRP-M and PIRP-L also described below. These flanking sequences also provide a SacI restriction site at the 5′ end and a SacII site at the 3′ end for use in cloning. These flanking sequences are shown in the optimized and His-tagged sequences given below.

The nuelcotide and amino acid sequences of optimized PIRP-M are shown below and are SEQ ID NO:9 and SEQ ID NO:10, respectively. The nucleotide sequence is annotated and explained below.

```
PIRP-M (optimized)
−10 1 15 30 45
GAGCTCCACC ATG TAC GGT GTT CTC CCT TGG AAC GCT TTC CCT GGC AAG GTT TGT
                        Met Tyr Gly Val Leu Pro Trp Asn Ala Phe Pro Gly Lys Val Cys
                                  60 75 90
GGC TCC AAC CTT CTG TCC ATC TGC AAA ACA GCC GAG TTC CAA ATG ACC TTC CAC
Gly Ser Asn Leu Leu Ser Ile Cys Lys Thr Ala Glu Phe Gln Met Thr Phe His
        105 120 135 150
CTG TTT ATT GCT GCG TTT GTG GGT GCT GCG GCC ACA CTA GTT TCC CTG CTC ACC
Leu Phe Ile Ala Ala Phe Val Gly Ala Ala Ala Thr Leu Val Ser Leu Leu Thr
                        165 180 195
TTC ATG ATT GCT GCC ACT TAC AAC TTC GCC GTC CTT AAA CTC ATG GGC CGA GGC
Phe Met Ile Ala Ala Thr Tyr Asn Phe Ala Val Leu Lys Leu Met Gly Arg Gly
210 225
ACC AAG TTC TGA CCG CGG
Thr Lys Phe ***
```

Unlike the earlier sequences, here the coding sequence is numbered beginning at position 1 for the A or the ATG start codon. The 5′ flanking sequence GAGCTCCACC is numbered from −10 to −1. The 3′ flanking sequence CCGGCC is numbered sequentially 220-225. In addition, in this as well as the other optimized sequences shown below, three nucleotides of the coding sequence were chaged, as shown underscored and boldfaced (C⁶, T²¹ and C²⁷. These changes optimized the sequence in that they did not change the encoded amino acid but they eliminated any downstream ATG codons so that translation must begin from the 1^{st} start codon.

The sequence below is the His-tagged PIRP-M insert showing a coding sequence that is the same as that shown above but includes a run of 6 His codons at the 3′ end. As is well known in the art, the His is added to provide a “tail” that can be bound by certain affinity probes (here, a Nickel column) for purposes of isolation and purification. The His residues and their codons are underscored. PIRP-M-His (nt sequence is SEQ ID NO:11 and amino acid sequence is SEQ ID NO:12)

```
−10 1 15 30 45
GAGCTCCACC ATG TAC GGT GTT CTC CCT TGG AAC GCT TTC CCT GGC AAG GTT TGT
                        Met Tyr Gly Val Leu Pro Trp Asn Ala Phe Pro Gly Lys Val Cys
                                  60 75 90
GGC TCC AAC CTT CTG TCC ATC TGC AAA ACA GCC GAG TTC CAA ATG ACC TTC CAC
Gly Ser Asn Leu Leu Ser Ile Cys Lys Thr Ala Glu Phe Gln Met Thr Phe His
        105 120 135 150
CTG TTT ATT GCT GCG TTT GTG GGT GCT GCG GCC ACA CTA GTT TCC CTG CTC ACC
Leu Phe Ile Ala Ala Phe Val Gly Ala Ala Ala Thr Leu Val Ser Leu Leu Thr
                        165 180 195
TTC ATG ATT GCT GCC ACT aTAC AAC TTC GCC GTC CTT AAA CTC ATG GGC CGA GGC
Phe Met Ile Ala Ala Thr Tyr Asn Phe Ala Val Leu Lys Leu Met Gly Arg Gly
210 225 240
ACC AAG TTC CAT CAT CAC CAT CAC CAT TGA CCG CGG
Thr Lys Phe His His His His His His ***
```

The nuelcotide and amino acid sequences of optimized PIRP-L are shown below and are SEQ ID NO: 13 and SEQ ID NO:14, respectively. The nucleotide sequence is annotated and explained below. The ‘5 and 3’ untranslated sequences are as shown above.

```
PIRP-L (Optimized)
−10 1 15 30 45
GAGCTCCACC ATG ACC TTC CAC CTG TTT ATT GCT GCG TTT GTG GGT GCT GCG GCC
                        Met Thr Phe His Leu Phe Ile Ala Ala Phe Val Gly Ala Ala Ala
        105 120 135 150
ACA CTA GTT TCC CTG CTC ACC TTC ATG ATT GCT GCC ACT TAC AAC TTC GCC GTC
Thr Leu Val Ser Leu Leu Thr Phe Met Ile Ala Ala Thr Tyr Asn Phe Ala Val
                        165 180
CTT AAA CTC ATG GGC CGA GGC ACC AAG TTC TGA CCG CGG
Leu Lys Leu Met Gly Arg Gly Thr Lys Phe ***
```

The sequence below is the His-tagged PIRP-M insert showing a coding sequence that is the same as that shown above but includes a run of 6 His codons at the 3′ end. As is well known in the art, the His is added to provide a “tail” that can be bound by certain affinity probes (here, a Nickel column) for purposes of isolation and purification. The His residues and their codons are underscored.

```
PIRP-L-His (nt sequence is SEQ ID NO:15 and
amino acid sequence is SEQ ID NO:16)
−10 15 30 45
GAGCTCCACC ATG ACC TTC CAC CTG TTT ATT GCT GCG TTT GTG GGT GCT GCG GCC
                        Met Thr Phe His Leu Phe Ile Ala Ala Phe Val Gly Ala Ala Ala
        105 120 135 150
ACA CTA GTT TCC CTG CTC ACC TTC ATG ATT GCT GCC ACT TAC AAC TTC GCC GTC
Thr Leu Val Ser Leu Leu Thr Phe Met Ile Ala Ala Thr Tyr Asn Phe Ala Val
                        165 180 195
CTT AAA CTC ATG GGC CGA GGC ACC AAG TTC CAT CAT CAC CAT CAC CAT TGA CCG
Leu Lys Leu Met Gly Arg Gly Thr Lys Phe His His His His His His ***
210
CGG
```

The PIRP present in the jimpy mouse mutant, termed PIRP-J, is shown below, and differs substantially in sequence from the wild-type or optimzed PIRP-M and PIRP-L sequences. The nucleotide sequence is SEQ ID NO:17 and includes a coding sequnce and a 5′ and 3′ untranslated region. The amino acid is SEQ ID NO:18:

```
PIRP-J:
−10 1 15 30 45
GAGCTCCACC ATG TAT GTT CCA AAT GAC CTT CCA CCT GTT TAT TGC TGC GTT TGT
                        Met Tyr Val Pro Asn Asp Leu Pro Pro Val Tyr Cys Cys Val Cys
                                  60 75 90
GGG TGC TGC GGC CAC ACT AGT TTC CCT GCT CAC CTT CAT GAT TGC TGC CAC TTA
Gly Cys Cys Gly His Thr Ser Phe Pro Ala His Leu His Asp Cys Cys His Leu
        105 130 140 150
CAA CTT CGC CGT CCT TAA ACT CATGGGCCGA GGCACCAAGT TCTGACCGCG G
Gln Leu Arg Arg Pro *
```

The present invention is also directed to the IRES in the mRNA molecules described hereein The IRES of the present invention are useful in vectors for use in, for example, cell or gene therapy. Such vectors can be rendered specific for oligodendrocytes either by the choice of expression control elements or by physical or chemically- targeting, for example, route of administration (see discussion below for polypeptide administration). These IRES elements, because of their activation in the context of cell death or stress, can be targeted as vectors to dying cells prior to irreversible cell damage, where they may express the desired proteins in the apoptotic (or stressed) cell,

PLP/DM20-Coding Nucleic Acid and Polypeptide Product

The PIRP polypeptides of the present invention may be produced using conventional recombinant methods and, alternatively, by chemical synthesis. Such methods are well-known in the art and need not be repeated here. Appropriate mammalian, other eukaryotic and prokaryotic expression systems are well-known in the art.

Basic texts disclosing general methods of molecular biology, all of which are incorporated by reference, include: Sambrook, J et al., Molecular Cloning: A Laboratory Manual, 2^{nd} Edition, Cold Spring Harbor Press, Cold Spring Harbor, N.Y., 1989; Ausubel, F M et al. Current Protocols in Molecular Biology, Vol. 2, Wiley-Interscience, New York, (current edition); Kriegler, Gene Transfer and Expression: A Laboratory Manual (1990); Glover, D M, ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J D et al., Recombinant DNA, 2^{nd} Ed., Scientific American Books, New York, 1992; and Old, R W et al., Principles of Gene Manipulation. An Introduction to Genetic Engineering, 2^{nd} Ed., University of California Press, Berkeley, Calif. (1981).

Unless otherwise indicated, a particular nucleic acid sequence is intended to encompasses conservative substitution variants thereof (e.g., degenerate codon substitutions) and a complementary sequence. The term “nucleic acid” is synonymous with “polynucleotide” and is intended to include a gene, a cDNA molecule, an mRNA molecule, as well as a fragment of any of these such as an oligonucleotide, and further, equivalents thereof (explained more fully below). Sizes of nucleic acids are stated either as kilobases kb) or base pairs (bp). These are estimates derived from agarose or polyacrylamide gel electrophoresis (PAGE), from nucleic acid sequences which are determined by the user or published.

Protein size is stated as molecular mass in kilodaltons (kDa) or as length (number of amino acid residues). Protein size is estimated from PAGE, from sequencing, from presumptive amino acid sequences based on the coding nucleic acid sequence or from published amino acid sequences.

This invention includes isolated nucleic acids (=polynucleotides) derived from a natural source or of synthetic origin and having a nucleotide sequence encoding the novel PIRP-M or PIRP-L polypeptides, active fragments thereof or homologues or analogues thereof. The term nucleic acid or polynuelcotide, as used herein is can be DNA or RNA, and are intended to include such fragments, homologues, analogues, or equivalents.

A preferred nucleic acid is DNA having the sequence SEQ ID NO:5, 7, 9, 11, 13, 15 or 17 or equivalents thereof. Another preferred nucleic acid is an mRNA (unmodified or stabilized) encoded thereby.

The DNA can be made from mRNA extracted from cells, preferably human cells, naturally expressing these PIRP polypeptides, or from genomic DNA of such cells. Thus, the present DNA can be cloned from a cDNA or a genomic library in accordance with known protocols.

Fragment of the Nucleic Acid

A fragment of the nucleic acid sequence is defined as a nucleotide sequence having fewer nucleotides than the nucleotide sequence encoding the full length PIRP-L or PIRP-M proteins described above. This invention includes such nucleic acid fragments that encode polypeptides which retain the ability to stimulate growth or survival of oligodendrocytes, preferably human oligodendrocytes, in vitro or in vivo or to bind to the natural ligand(s) for PLP/DM20 protein or biologically active fragments thereof on oligodendrocytes or on other cells.

Generally, the nucleic acid sequence encoding a fragment of the PIRP-M, PIRP-L, or PIRP-J polypeptide comprises of nucleotides from the sequence encoding the mature protein. However, in some instances it may be desirable to include all or part of the leader sequence portion of the nucleic acid. Nucleic acid sequences of this invention may also include linker sequences, natural or modified restriction endonuclease sites and other sequences that are useful for manipulations related to cloning, expression or purification of encoded protein or fragments. These and other modifications of nucleic acid sequences are described herein or are well-known in the art.

The techniques for assembling and expressing DNA encoding the present proteins or fusion proteins thereof such as synthesis of oligonucleotides, PCR, transforming or transfecting cells, constructing vectors, expression systems, and the like are well-established in the art as indicated above. Those of ordinary skill are familiar with the standard resource materials for specific conditions and procedures.

In other embodiments, the DNA is a homologue that encodes another protein or a domain or fragment of another protein (termed a “fusion partner”). Preferred fusion proteins have been described in the Examples, particularly ones wherein the fusion partner acts as a tag or detectable label. Preferred fusion partners are Green Fluorescent Protein (GFP), either green or yellow GFP.

Additional fusion partners include a targeting moiety that helps target the polypeptide to cells or tissue of interest. Examples of these are antibody chains with antigen-binding activity, such as single chain antibodies (scFv molecules) (Skerra, A. et al. (1988) Science, 240: 1038-1041; Pluckthun, A. et al. (1989) Methods Enzymol. 178: 497-515; Winter, G. et al. (1991) Nature, 349: 293-299); Bird et al., (1988) Science 242:423; Huston et al. (1988) Proc. Natl. Acad. Sci. USA 85:5879; Jost C R et al,. J Biol Chem. 1994 269:26267-26273; U.S. Pat. Nos. 4,704,692, 4,853,871, 4,946,778, 5,260,203, 5,455,030).

Prokaryotic or eukaryotic host cells transformed or transfected to express the PIRP polypeptides of the present invention or a homologue or functional derivative thereof are within the scope of the invention. For example, the PIRP-L or PIRP-M polypeptide may be expressed in bacterial cells such as E. coli, insect cells (baculovirus), yeast, or mammalian cells such as Chinese hamster ovary cells (CHO) or human cells. Other suitable host cells may be found in Goeddel, (1990) supra or are otherwise known to those skilled in the art.

Expression in eukaryotic cells leads to partial or complete glycosylation and/or formation of relevant inter- or intra-chain disulfide bonds of the recombinant protein.

Examples of vectors for expression in yeast S. cerevisiae include pYepSec1 (Baldari et al., (1987) EMBO J. 6:229-234), pMFa (Kurjan et al. (1982) Cell 30:933-943), pJRY88 (Schultz et al., (1987) Gene 54:113-123), and pYES2 (Invitrogen Corporation, San Diego, Calif.). Baculovirus vectors available for expression of proteins in cultured insect cells (SF 9 cells) include the pAc series (Smith et al., (1983) Mol. Cell Biol. 3: 2156-2165,) and the pVL series (Lucklow, V. A., and Summers, M. D., (1989) Virology 170: 31-39). Generally, COS cells (Gluzman, Y., (1981) Cell 23: 175-182) are used in conjunction with such vectors as pCDM 8 (Aruffo A. and Seed, B., supra, for transient amplification/expression in mammalian cells, while CHO (dhfr-negative CHO) cells are used with vectors such as pMT2PC (Kaufman et al. (1987), EMBO J. 6: 187-195) for stable amplification/expression in mammalian cells. The NSO myeloma cell line (a glutamine synthetase expression system) is available from Celltech Ltd.

Often, in fusion expression vectors, a proteolytic cleavage site is introduced at the junction of the reporter group and the target protein to enable separation of the target protein from the reporter group subsequent to purification of the fusion protein. Proteolytic enzymes for such cleavage and their recognition sequences include Factor Xa, thrombin and enterokinase.

Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase, maltose E binding protein, or protein A, respectively, to the target recombinant protein.

Inducible non-fusion expression vectors include pTrc (Amann et al., (1988) Gene 69: 301-315) and pET 11 d (Studier et al., Gene Expression Technology: Methods in Enzymology 185, Academic Press, San Diego, Calif. (1990) 60-89). While target gene expression relies on host RNA polymerase transcription from the hybrid trp-lac fusion promoter in pTrc, expression of target genes inserted into pET 11 d relies on transcription from the T7 gn10-lacO fusion promoter mediated by coexpressed viral RNA polymerase (T7 gn1). Th is viral polymerase is supplied by host strains BL21(DE3) or HMS 174(DE3) from a resident λ prophage harboring a T7 gn1 under the transcriptional control of the lacUV 5 promoter.

One embodiment of this invention is a transfected cell which expresses the novel polypeptide of this invention de novo. In the case of a cell already expressing these products, the transfected cell expresses increased amounts of these proteins or fragments thereof on the cell surface or intracellularly.

Promoters and Enhancers

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. Two sequences of a nucleic acid molecule, such as a promoter and a coding sequence, are “operably linked” when they are linked to each other in a manner which permits both sequences to be transcribed onto the same RNA transcript or permits an RNA transcript begun in one sequence to be extended into the second sequence. Thus, two sequences, such as a promoter sequence and a coding sequence of DNA or RNA are operably linked if transcription commencing in the promoter sequence will produce an RNA transcript of the operably linked coding sequence. In order to be “operably linked” it is not necessary that two sequences be immediately adjacent to one another in the linear sequence.

The preferred promoter sequences of the present invention must be operable in mammalian cells and may be either eukaryotic or viral promoters. Suitable promoters may be inducible, repressible or constitutive. An example of a constitutive promoter is the viral promoter MSV-LTR, which is efficient and active in a variety of cell types, and, in contrast to most other promoters, has the same enhancing activity in arrested and growing cells. Other preferred viral promoters include that present in the CMV-LTR (from cytomegalovirus) (Bashart, M. et al., Cell 41:521 (1985)) or in the RSV-LTR (from Rous sarcoma virus) (Gorman, C. M., Proc. Natl. Acad. Sci. USA 79:6777 (1982). Also useful are the promoter of the mouse metallothionein I gene (Hamer, D. et al., J. Mol. Appl. Gen. 1:273-288 (1982)), the TK promoter of Herpes virus (McKnight, S., Cell 31:355-365 (1982)); the SV40 early promoter (Benoist, C. et al., Nature 290:304-310 (1981)); and the yeast gal4 gene promoter (Johnston, S. A. et al., Proc. Natl. Acad Sci. USA 79:6971-6975 (1982); Silver, P. A. et al., Proc. Natl. Acad. Sci. USA 81:5951-5955 (1984)). Other illustrative descriptions of transcriptional factor association with promoter regions and the separate activation and DNA binding of transcription factors include: Keegan et al., Nature (1986) 231:699; Fields et al., Nature (1989) 340:245; Jones, Cell (1990) 61:9; Lewin, Cell (1990) 61:1161; Ptashne et al., Nature (1990) 346:329; Adams et al., Cell (1993) 72:306. The relevant disclosure of all of these above-listed references is hereby incorporated by reference.

The promoter region may further include an octamer region which may also function as a tissue specific enhancer, by interacting with certain proteins found in the specific tissue. The enhancer domain of the DNA construct of the present invention is one which is specific for the target cells to be transfected, or is highly activated by cellular factors of such target cells. Examples of vectors (plasmid or retrovirus) are disclosed in (Roy-Burman et al., U.S. Pat. No. 5,112,767). For a general discussion of enhancers and their actions in transcription, see, Lewin, B. M., GENES IV, Oxford University Press, Oxford, (1990), pp. 552-576. Particularly useful are retroviral enhancers (e.g., viral LTR). The enhancer is preferably placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency on the coding DNA molecule of the present invention.

The nucleic acid sequences of the invention can also be chemically synthesized using standard techniques. Various methods of chemically synthesizing polydeoxynucleotides are known, including solid-phase synthesis which, like peptide synthesis, has been fully automated with commercially available DNA synthesizers (See, e.g., Itakura et al U.S. Pat. No. 4,598,049; Caruthers et al. U.S. Pat. No.4,458,066; and Itakura U.S. Pat. Nos. 4,401,796 and 4,373,071, incorporated by reference herein).

Proteins and Polypeptides

The present invention includes an “isolated” PIRP-L or PIRP-M polypeptides having the human sequences SEQ ID NO:6 or SEQ ID NO:8. It is to be understood that homologues from other mammalian species and mutants thereof that possess the characteristics disclosed herein are intended within the scope of this invention.

Also included is a “functional derivative” of these polypeptides which means an amino acid substitution variant, a “fragment,” or a “chemical derivative” (which terms are defined below). A functional derivative retains measurable activity of the “parent” sequence, preferably that of stimulating growth of oligodendrocytes (in the case of the PIRP-M protein) or regulating the activity of the PIRP-M protein (in the case of the PIRP-L protein). Such regulation may occur by hydrophobic interactions of the TM domains common to both molecules. Any activity which permits their utility in accordance with the present invention is intended. “Functional derivatives” encompass “variants” and “fragments” regardless of whether the terms are used in the conjunctive or the alternative herein.

A functional homologue must possess the above biochemical and biological activity. Because of the strong conservation of these sequences (discussed herein), any homologous polypeptide from another species falls within the scope of the invention.

For synthetic peptides or molecules yet undiscovered from other species, one determines the percent identity of two amino acid sequences or of two nucleic acid sequences by aligning them for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned.

In a preferred embodiment, the length of a sequence being compared is at least 30%, preferably at least 40%, more preferably at least 50%, even more preferably at least 60%, and even more preferably at least 70%, 80%, or 90% of the length of the reference sequence. The amino acid residues (or nucleotides) at corresponding amino acid positions (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to human nucleic acid sequences encoding PLP LMW polypeptides. BLAST protein searches can be performed with the XBLAST program, score=50, wordlength=3 to obtain amino acid sequences homologous to the native protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Thus, a homologue of the PIRP polypeptide described above is characterized as having (a) functional activity of native polypeptide, and (b) sequence similarity to a native polypeptide (such as SEQ ID NO:6 or SEQ ID NO:8, when determined above, of at least about 30% (at the amino acid level), preferably at least about 50%, more preferably at least about 70%, even more preferably at least about 90%.

It is within the skill in the art to obtain and express such a protein using DNA probes based on the disclosed sequences. Then, the protein's biochemical and biological activity can be tested readily using art-recognized methods such as those described herein, for example, a standard oligodendrocyte proliferation or survival assay using cell lines or primary cells (or cells is/ vivo) to indicate whether the homologue has the requisite activity to qualify as a “functional” homologue.

A “variant” of the reference polypeptide refers to a molecule substantially identical to either the full protein or to a fragment thereof in which one or more amino acid residues have been replaced (substitution variant) or which has one or several residues deleted (deletion variant) or added (addition variant). A “fragment” refers to any subset of the molecule that is, a shorter polypeptide of, for example, SEQ ID NO:6 or SEW ID NO:8.

A number of processes can be used to generate fragments, mutants and variants of the isolated DNA sequence. Small subregions or fragments of the nucleic acid can be prepared by standard, chemical synthesis.

A preferred functional derivative is a fusion protein, a polypeptide that includes a functional fragment of the PLP polypeptide. As noted above, fusion proteins with peptide or polypeptides sequences that serve as markers, tags for purification or as targeting structures (e.g., scFv polypeptides) are preferred.

By “soluble PLP LMW polypeptide” is intended a cell-free form of the polypeptide that may be shed, secreted or otherwise extracted from the producing cells. This includes, but is not limited to, soluble fusion proteins.

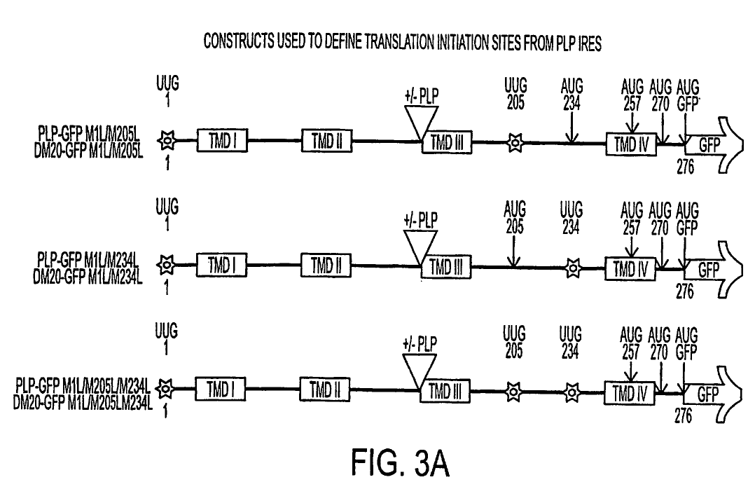

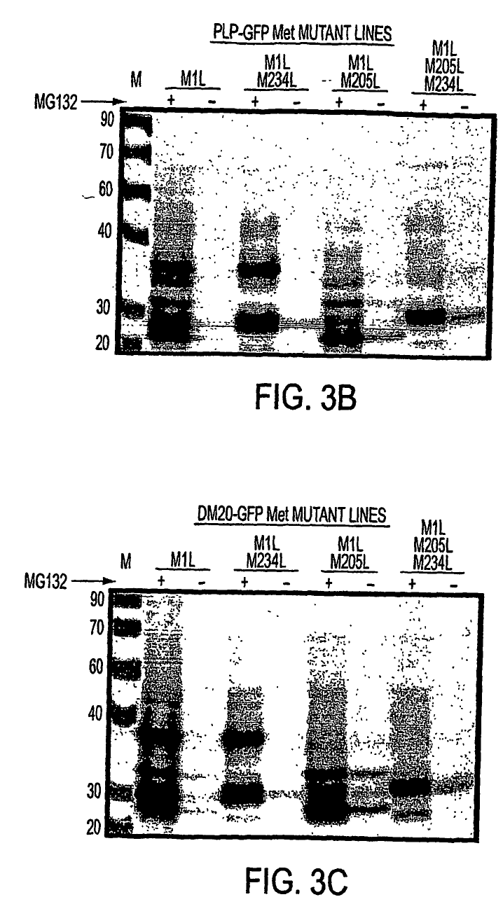

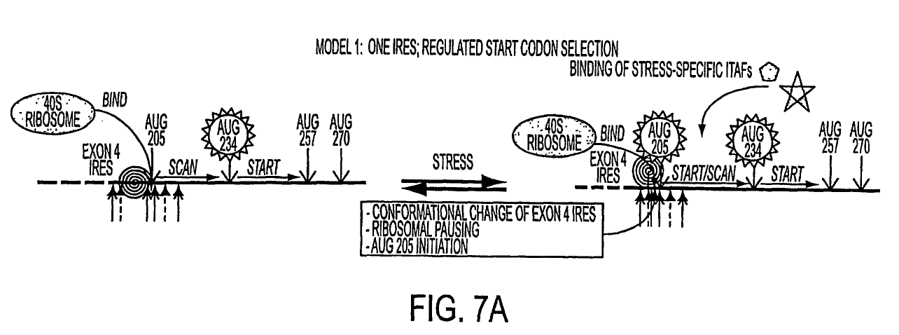

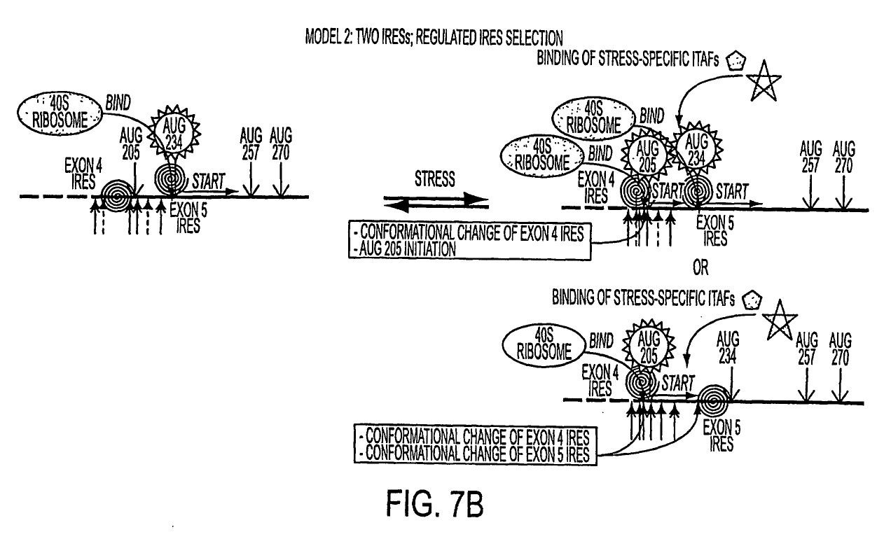

A preferred group of variants are those in which at least one amino acid residue and preferably, only one, has been substituted by different residue. For a detailed description of protein chemistry and structure, see Schulz, G E et al., Principles of Protein Structure, Springer-Verlag, New York, 1978, and Creighton, T. E., Proteins: Structure and Molecular Properties, W. H. Freeman & Co., San Francisco, 1983, which are hereby incorporated by reference. The types of substitutions that may be made in the protein molecule may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

| | | |
|---|---|---|
| 1 | Small aliphatic, nonpolar or slightly polar | Ala, Ser, Thr (Pro, Gly); |
| | residues | |
| 2 | Polar, negatively charged residues and | Asp, Asn, Glu, Gln; |
| | their amides | |
| 3 | Polar, positively charged residues | His, Arg, Lys; |
| 4 | Large aliphatic, nonpolar residues | Met, Leu, Ile, Val (Cys) |
| 5 | Large aromatic residues | Phe, Tyr, Trp. |

The three amino acid residues in parentheses above have special roles in protein architecture. Gly is the only residue lacking a side chain and thus imparts flexibility to the chain. Pro, because of its unusual geometry, tightly constrains the chain. Cys can participate in disulfide bond formation, which is important in protein folding.

More substantial changes in biochemical, functional (or immunological) properties are made by selecting substitutions that are less conservative, such as between, rather than within, the above five groups. Such changes will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ile, Phe, Val or Ala; (iii) substitution of a Cys residue for (or by) any other residue; (iv) substitution of a residue having an electropositive side chain, e.g., Lys, Arg or His, for (or by) a residue having an electronegative charge, e.g., Glu or Asp; or (v) substitution of a residue having a bulky side chain, e.g., Phe, for (or by) a residue not having such a side chain, e.g., Gly.

Most acceptable deletions, insertions and substitutions according to the present invention are those that do not produce radical changes in the characteristics of the B7-DC protein in terms of its T cell costimulatory activity. However, when it is difficult to predict the exact effect of the substitution, deletion or insertion in advance of doing so, one skilled in the art will appreciate that the effect can be evaluated by routine screening assays such as those described here, without requiring undue experimentation.

Whereas shorter chain variants can be made by chemical synthesis, for the present invention, the preferred longer chain variants are typically made by site-specific mutagenesis of the nucleic acid encoding the PIRP polypeptide, expression of the variant nucleic acid in cell culture, and, optionally, purification of the polypeptide from the cell culture, for example, by immunoaffinity chromatography using specific antibody immobilized to a column (to absorb the variant by binding to at least one epitope).

Chemical Derivatives of the PIRP Polypeptide

“Chemical derivatives” of these molecules contain additional chemical moieties not normally a part of the protein. Covalent modifications of the polypeptide are included within the scope of this invention. Such derivatized moieties may improve the solubility, absorption, biological half life, and the like. Moieties capable of mediating such effects are disclosed, for example, in Remington's Pharmaceutical Sciences, 16^{th} ed., Mack Publishing Co., Easton, Pa. (1980).

Such modifications may be introduced into the molecule by reacting targeted amino acid residues of the polypeptide with an organic derivatizing agent that is capable of reacting with selected side chains or terminal residues. Another modification is cyclization of the protein.

Examples of chemical derivatives of the polypeptide follow.

Lysinyl and amino terminal residues are derivatized with succinic or other carboxylic acid anhydrides. Derivatization with a cyclic carboxylic anhydride has the effect of reversing the charge of the lysinyl residues. Other suitable reagents for derivatizing amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Carboxyl side groups, aspartyl or glutamyl, may be selectively modified by reaction with carbodiimides (R—N═C═N—R′) such as 1-cyclohexyl-3-(2-morpholinyl-(4-ethyl) carbodiimide or 1-ethyl-3-(4-azonia4,4-dimethylpentyl) carbodiimide. Furthermore, aspartyl and glutamyl residues can be converted to asparaginyl and glutaminyl residues by reaction with ammonia.

Other modifications include hydroxylation of proline and lysine, phosphorylation of hydroxyl groups of seryl or threonyl residues, methylation of the amino group of lysine (Creighton, supra, pp. 79-86 ), acetylation of the N-terminal amine, and amidation of the C-terminal carboxyl groups.

Also included are peptides wherein one or more D-amino acids are substituted for one or more L-amino acids.

Pharmaceutical and Therapeutic Compositions and Their Administration

The polypeptides of this invention or a cell expressing this polypeptide (or a cell expressing the IRES) is administered to a mammalian subject, preferably a human in need of such treatment.

The PIRP-M polypeptide or a functional derivative is useful for the treatment of any disorder where remyelination or stimulation of oligodendroglia or Schwann cells is desirable. This includes any of a number of demyelinating or dysmyelintation diseases, including multiple sclerosis (MS), closed head trauma associated with Parkinson's-like symptoms, hypoxic ischemia such as that associated with surgery, or spinal cord trauma.

The PIRP-M polypeptide or a functional derivative is useful for stimulating neural stem cells at various stages of differentiation or commitment, and promoting their differentiation, maturation along the oligodendrocytic cell lineage and for proliferation of the oligodendrocytes and their precursors. Similarly, the polypeptides act to protect oligodendrocytes (or other cells, including non-neural cells) from apoptotic death. These agents are therefore useful for treating any diseases in which such differentiation, maturation and proliferation or inhibition of cell death is palliative or curative.

Once receptors for these molecules have been identified, they can be harnessed to increase selectivity and efficiency of delivery of the polypeptides to the cells, enhance their internalization in an environment in which the polypeptide acts in an intracrine manner.

The PIRP-L polypeptide is useful due to its ability to regulate or inhibiting the action of PLP/DM20 or of the PIRP-M polypeptide fragment if and under conditions that PIRP-M is naturally (i.e., pathogenically) produced. Preferred examples of this are brain tumors, particularly oligodendrogliomas which are major killers, but also various benign glial tumors. This shorter fragment is expected to shut down production of the PIRP-M molecule in vivo.

The present polypeptides are intended to be used alone or in combination with conventional drugs or biologics know to be effective or partially effective in treating the appropriate disease or conditions. Thus, in the case of MS, a preferred embodiment is a therapeutic composition (and method) comprising the PIRP-M polypeptide described herein, or a functional derivative thereof, in combination with an “ABC” drug (acyclovir, betaseron®, copaxone®).

A composition comprising the PIRP-M polypeptide or derivative, is administered in a pharmaceutically acceptable carrier in a biologically effective in a therapeutically effective amount, either alone or in combination with another agent.

A therapeutically effective amount is a dosage that, when given for an effective period of time, achieves the desired neurological or clinical effect.

A therapeutically active amount of the polypeptide may vary according to factors such as the disease state, age, sex, and weight of the individual, and the ability of the peptide to elicit a desired response in the individual. Dosage regimes may be adjusted to provide the optimum therapeutic response. For example, several divided doses may be administered daily or the dose may be proportionally reduced as indicated by the exigencies of the therapeutic situation. A therapeutically effective amounts of the protein, in cell associated form may be stated in terms of the protein or cell equivalents.

Thus an effective amount is between about 1 ng and about 1 gram per kilogram of body weight of the recipient, more preferably between about 1 μg and 100 mg/kg, more preferably, between about 100 μg and about 100 mg/kg. Dosage forms suitable for internal administration preferably contain (for the latter dose range) from about 0.1 mg to 500 mg of active ingredient per unit. The active ingredient may vary from 0.5 to 95% by weight based on the total weight of the composition. Alternatively, an effective dose of cells expressing the polypeptide is between about 10⁴ and 10⁹ cells, more preferably between about 10⁶ and 10⁸ cells per subject, preferably in split doses. Those skilled in the art of cell therapy will be able to adjust these doses without undue experimentation.

The active compound (e.g., the polypeptide or cell transduced with encoding DNA) may be administered in a convenient manner, e.g., by injection or infusion, by a convenient and effective route. Preferred routes include intravenous, intrathecal, intracerebroventricular, subcutaneous, intradermal, and intramuscular routes. Other possible routes include oral administration, inhalation, or rectal administration. For the treatment of tumors which have not been completely resected, direct intratumoral injection is also intended.

Depending on the route of administration, the active compound may be coated in a material to protect the compound from the action of enzymes, acids and other natural conditions which may inactivate the compound. Thus, to a administer a polypeptide or peptide by an enteral route, it may be necessary to coat the composition with, or co-administer the composition with, a material to prevent its inactivation. For example, a peptide may be administered to an individual in an appropriate carrier, diluent or adjuvant, co-administered with enzyme inhibitors (e.g., pancreatic trypsin inhibitor, diisopropylfluorophosphate (DEP) and trasylol) or in an appropriate carrier such as liposomes (including water-in-oil-in-water emulsions as well as conventional liposomes (Strejan et al., (1984) J. Neuroimmunol 7:27).

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Preferred pharmaceutically acceptable diluents include saline and aqueous buffer solutions. Pharmaceutical compositions suitable for injection include sterile aqueous solutions (where water soluble) or dispersions and sterile powders for the extemporaneous preparation of sterile injectable solutions or dispersion. Isotonic agents, for example, sugars, polyalcohols such as mannitol, sorbitol, sodium chloride may be included in the pharmaceutical composition. In all cases, the composition should be sterile and should be fluid. It should be stable under the conditions of manufacture and storage and must include preservatives that prevent contamination with microorganisms such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal, and the like.

Prolonged absorption of the injectable compositions can be brought about by including in the composition an agent which delays absorption, for example, aluminum monostearate and gelatin.

Parenteral compositions are preferably formulated in dosage unit form for ease of administration and uniformity of dosage. Dosage unit form refers to physically discrete units suited as unitary dosages for a mammalian subject; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

In a sprayable aerosol preparations, the active protein may be in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

Other pharmaceutically acceptable carriers for the polypeptide according to the present invention are liposomes, pharmaceutical compositions in which the active protein is contained either dispersed or variously present in corpuscles consisting of aqueous concentric layers adherent to lipidic layers. The active protein is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature.

Reagent/Diagnostic Compositions and Methods

The present polypeptide have additional uses, including as reagents in methods for isolating ligands or binding partners for these polypeptides or epitopes thereof, including antibodies, cellular receptors, and the like. Thus, the present invention includes assays, including immunoassays, which can be used in the research setting or a diagnostic setting to determine the presence or measure the levels of an agent which, for example, binds to the PIRP-M or PIRP-L fragment. The present invention is not intended to be limited to therapeutic uses of the compositions disclosed herein.

For example, the PIRP-M polypeptide, that includes a growth factor activity, can be used to screen for receptors for the growth factor sequence. To the extent that the PIRP-L and PIRP-M polypeptide interact, presumably via their hydrophobic regions to form transient or stable dimers (or higher oligomers), either or both of these agents can be used in assays to screen for compounds that either inhibit or promote that interaction.

Having now generally described the invention, the same will be more readily understood through reference to the following examples which are provided by way of illustration, and are not intended to be limiting of the present invention, unless specified.

### EXAMPLE I

### Materials and Methods

Cloning PLP and DM20 cDNA Sequences into Mammalian Expression Vectors.

The vectors are described in Table 1. Information about PCR primers can be found in Table 2.

TABLE 1
Parental Vector Information Summary.

| | Genbank | | | Protein | Antibiotic resistance | |
|---|---|---|---|---|---|---|
| Vector | Accession # | Type | Promoter | tag | Mammalian | Bacterial |
| pCMV | — | Mammalian | constitutive | None | G418 | Kan¹ |
| | | expression | CMV | | | |
| pEGFP-N1 | U55762 | mammalian | constitutive | green | G418 | Kan |
| | | expression | CMV | GFP² | | |
| pEYFP-N1 | U55762 | mammalian | constitutive | yellow | G418 | Kan |
| | | expression | CMV | GFP | | |
| pEGFP-Tet-On | — | Tet response | Tet-inducible | green | G418 | Kan |
| | | mammalian | bidirectional | GFP | | |
| | | expression | CMV-Tet-On | | | |
| pBluescript II | X52328 | bacterial | T7 promoter | none | none | Amp⁴ |
| KS+ | | expression/ | | | | |
| | | in vitro | | | | |
| | | expression | | | | |
| pET-14b | — | bacterial | IPTG⁵ - | 6XHis | none | Amp |
| | | expression | inducible T7 | | | |
| | | | promoter | | | |

All GFP tagged vectors except “pEGFP-Tet-On” were purchased from Clontech. pBluescript II KS+ and pET-14b vectors were purchased from Stratagene and Novagen, respectively. pEGFP-Tet-On was constructed by replacing the CMV promoter cassette in pEGFP-N1 with the bidirectional Tet inducible CMV-Tet-On promoter cassette from pBI vector, Genbank accession # U89932 (Clontech).
pCMV vector was derived from pGFP-C3 vector (Clontech) by removing the GFP coding sequence.
¹Kan = kanamycin
²GFP = green fluorescent protein
³Tet = tetracycline
⁴Amp = Ampampicillin
⁵IPTG = Isopropyl-1-thio-β-D-galactopyranoside

pCMV Constructs Containing the Proteolipid cDNAs

The full length wt and jimpy mouse PLP/DM20 cDNA's (NPLP, NDM20, JPLP, and JDM20) were cloned into the BamHI site of pGEM 7Z vector (provided by Dr. A. Fannon, Mt. Sinai School of Medicine, New York). The inserts were released by BamHI digestion and the purified NPLP, NDM20, JPLP, and JDM20 cDNA's ligated into the BamH I site of the pCMV expression vector (Clontech), using T4 DNA ligase. The ligation products were transformed into competent LE392 cells (Stratagene). Competent LE392 cells were prepared using standard moelcular techniques or purchased from commercial vendors. Small scale plasmid preparations were produced from colonies using standard alkaline lysis protocol or via a Wizard® Plus Minipreps DNA Purification System (Promega) following the manufacturer's protocol. Insert orientation was verified by HindIII/SpeI digestion. Sense orientation clones were obtained for all four constructs and named pNPLP, pNDM20, pJPLP, and pJDM20. Sequence analysis of selected clones was performed at the WSU DNA Sequencing Facility.

Constructs Expressing Proteolipid EGFP Fusion Proteins.

Plasmids expressing the wt PLP/DM20 EGFP fusion proteins were prepared in the pCMV vector by insertion of the EGFP gene from the pEGFP-N1 vector (Clontech). A Sac I site in the 3′ end of the NPLP and NDM20 ORF's was used to insert the EGFP ORF and delete the proteolipid stop codon.

However prior to cloning the EGFP ORF, a Sac I site in the pCMV polylinker was removed. The pNPLP and pNDM20 vectors were cut with XbaI and EcoRI and incubated with 4 units of the large Klenow fragment of DNA polymerase I and 33,μM dNTPs (30 min, RT). Following phenol extraction and ethanol precipitation, the blunt ends were ligated using T4 DNA ligase. The ligation products were transformed into LE392 competent cells, selected for Kan resistance, and sized using gel electrophoresis. Wizard® Plus minipreps were scored for the absence of SacI and HindIII restriction sites in the polylinker. Positive constructs were named pΔ2 (PLP vector containing the polylinker deletion) and pΔ6 (a DM20 deletion construct).

pΔ2 and pΔ6 were digested at the unique Sac I site, which overlaps the PLP/DM20 termination codon. The linear products were purified by phenol extraction and ethanol precipitation, then blunted in the presence of 25 units of T4 DNA polymerase and 100 μM dNTPs (20 min, 12° C.) to delete the stop codon. The DNA fragments were purified and cut with MunI. In a separate reaction the pEGFP-N1 was cut at a unique NcoI site, located within the initiation codon of the EGFP ORF, and incubated with the Klenow fragment of DNA polymerase. Following phenol extraction and ethanol precipitation, the linear product was cut with MunI.

The pΔ2 and pΔ6 vector and the pEGFP fragment were gel purified and ligated using T4 DNA ligase. Ligation products were transformed into LE392 competent cells, selected for Kan resistance, and sized as described. To verify the integrity of the EGFP insertion, Wizards Plus minipreps were cut with SpeI/MunI and assayed for SacI resistance. In addition, positive constructs were DNA sequenced and named Δ2 EGFP and Δ6 EGFP.

Constructs Expressing Jimpy EGFP Fusion Proteins.

Since the Sac I site in the JPLP and JDM20 ORF's does not overlap the jimpy stop codon, a different strategy was used to generate the jimpy EGFP fusion clones. Jimpy sequences were PCR amplified from pJPLP and pJDM20 templates with PLP3 and BP2 primers (see Table 2).

The 50 μL PCR reactions contained 50 ng of template DNA, 250 ng of each primer, 1×reaction buffer (Buffer F or J, Invitrogen PCR Optimizer™ Kit), 1 unit Taq DNA polymerase (Qiagen), and 1 mM dNTPs (added during the 15 min 80° C. incubation, see below). The reactions were overlaid with 50 μL mineral oil, preincubated for 5 min 95° C., and a 15 min 80° C. incubation (during which the dNTPs were added). Amplification was performed using 35 cycles of 1 min at 94° C., 2 min at 55° C., and 3 min at 72° C. The JPLP/JDM20 amplification products were purified using Wizard® PCR Preps DNA Purification System (Promega), cut with BamHI and SacII, and purified using Wizard® DNA Clean-Up System (Promega). Both purification steps were performed as directed by Promega. The pEGFP-N1 plasmid was cut with Bgl II and Sac II, purified using the Wizard® DNA Clean-Up System (Promega), and ligated with the BamHI/SacII digested JPLP and JDM20 PCR products using T4 DNA ligase. The ligation products were transformed into LE392 competent cells, selected for Kan resistance, and sized as described vs. a pEGFP-N1 size control. To confirm the presence of the insert, Wizard® Plus minipreps were cut with NheI and SacII and positive clones were DNA sequenced and named JPLP-EGFP and JDM20-EGFP.

Large Scale Plasmid DNA Preparation.

All plasmid DNAs used for transfection experiments and site directed mutagenesis were isolated from LE392 or XL1-Blue bacteria using EndoFree Plasmid Maxi Kit (Qiagen) as directed by the manufacturer. This method is well-known in the art.

Cell Lines and Tissue Culture Conditions.

All constitutive expression studies were performed in three cell lines: HEK 293 (hereafter referred to as 293 cells), NTera2D (subsequently termed NT2 cells) and Cos-7 cells. The 293 cell line is a human embryonic kidney cell transformed with the adenovirus E1A gene (obtained from the AIDS Research and Reference Reagent Program, NIH, Bethesda, Md.). The NT2 cell line is a human teratocarcinoma cell capable of terminal differentiation into neurons in response to retinoic acid (purchased from Stratagene, La Jolla, Calif.). The Cos-7 cell line is a monkey fibroblast cell transformed with the SV40 T antigen, obtained from American Type Culture Collection (Manassas, Va.), ATCC CRL-1651 . All cells were maintained at 37° C., 5% CO₂ in Dulbecco's Modified Eagle Medium (DMEM) (Invitrogen Life Technologies), supplemented with 10% fetal bovine serum (Hyclone), 3.7 g/L sodium bicarbonate, and 30 mg/L gentamnicin sulfate (Invitrogen Life Technologies).

Transient Transfection Assays

Transfections were performed in 100×20 mm dishes using the Profection® Mammalian Transfection System—a calcium phosphate transfection kit (Promega, Madison, Wis.). Cells grown to 50-70% confluence were fed 3 hrs prior to addition of the calcium phosphate/DNA mixture. A standard transfection assay contained 15 μg of plasmid DNA, while double transfections were done with 10 μg of each plasmid (20 μg total). The culture medium was removed 24 hrs post transfection and cells were processed for microscopic analysis or passed for G418 or Hygromycin B selection 48 hrs post transfection.

For microscopic analysis, equal numbers of cells were passed into each well of a six-well tissue culture tray containing uncoated sterile glass coverslips. For time course studies, slides were removed and mounted every 24 hr, for six days after passage. For mounting, coverslips were rinsed in 1× PBS-T (1× PBS, 0.1% Tween 20), fixed in 4% paraformaldehyde (10 min, RT), rinsed in 1× PBS-T (twice), and attached to a slide using AquaPolyMount (Polysciences Inc., Warrington, Pa.).

TABLE 2
Primer Summary

| | | | | | SEQ |
|---|---|---|---|---|---|
| | | | | | ID |
| Primer | Type | Mutation | Linker | Sequence | NO: |
| PLP3 | PCR/ | (wt) | BamHI | 5′ CGGGATCCTCAGAGTGCCAAAGACATG 3′ | 19 |
| | Sequencing | | | | |
| PLP4 | PCR/ | Removes stop | Sac II | 5′ TTTCCGCGGGAACTTGGTGCCTCGGCC 3′ | 20 |
| | Sequencing | codon | | | |
| PLP4TGA | PCR/ | inserts stop | Sac II | 5′ TTTCCGCGGTCAGAACTTGGTGCCTCGGCC 3′ | 21 |
| | Sequencing | codon | | | |
| PLP4-His | PCR/ | inserts 6XHis tag | Sac II | 5′ TTTCCGCGGTCAATGGTGATGGTGATGATGGAACTTGGTGCCTCGGCC 3′ | 22 |
| | Mutagenic | and stop codon | | | |
| BP2 | PCR/ | Removes stop | Sac II | 5′ TTTCCGCGGAGGACGGCGAAGTTGTA 3′ | 23 |
| | Sequencing | codon | | | |
| | | (jimpy specific) | | | |
| MC5 | PCR/ | M1 < L* | EcoRI | 5′ CGGAATTCTCAGAGTGCCAAAGACAT 3′ | 24 |
| | Mutagenic | | | | |
| MC1 | PCR/ | K268 < R | Sac II | 5′ TCCCCGCGGGAACTTGGTGCCTCGGCCCATGAGTCTAAGGAC 3′ | 25 |
| | Mutagenic | | | | |
| MC2 | PCR/ | K275 < R | Sac II | 5′ TCCCCGCGGGAACCTGGTGCCTCGGCCCATGAGTTTAAGGAC 3′ | 26 |
| | Mutagenic | | | | |
| MC3 | PCR/ | K268 < R/ | Sac II | 5′ TCCCCGCGGGAACCTGGTGCCTCGGCCCATGAGTCTAAGGAC 3′ | 27 |
| | Mutagenic | K275 < R | | | |
| MC5 | PCR/ | M1 < L* | EcoRI | 5′ CGGAATTCTCAGAGTGCCAAAGACAT 3′ | 28 |
| | Mutagenic | | | | |
| MC7 | PCR/ | R272 < K | Sac II | 5′ TTTCCGCGGGAACTTGGTGCCTTTGCCCATGAG 3′ | 29 |
| | Mutagenic | | | | |
| MC5.5 | QuikChange | M1 < L | — | 5′ CCTCAGAGTGCCAAAGACTTGGGCTTGTTAGAGTG 3′ | 30 |
| MC5.3 | Mutagenic | | | 5′ CACTCTAACAAGCCCAAGTCTTTGGCACTCTGAGG 3′ | |
| L205.5w | QuikChange | M205 < L | — | 5′ CTGCGCTGATGCCAGATTGTATGGTGTTCTCCC 3′ | 31 |
| L205.3w | Mutagemc | (wt specific) | | 5′ GGGAGAACACCATACAATCTGGCATCAGCGCAG 3′ | |
| L205.5j | QuikChange | M205 < L | — | 5′ CTGCGCTGATGCCAGATTGTATGTTCCAAATGACCTTCC 3′ | 32 |
| L205.3j | Mutagenic | (jimpy specific) | | 5′ GGAAGGTCATTTGGAACATACAATCTGGCATCAGCGCAG 3′ | |
| L235.5 | QuikChange | M234 <M L | — | 5′ CTGCAAAACAGCTGAGTTCCAATTGACCTTCCACCTG 3′ | 33 |
| L235.3 | Mutagenic | | | 5′ CAGGTGGAAGGTCAATTGGAACTCAGCTGTTTTGCAG 3′ | |
| 205M | PCR/ | (see 1) | Sac I | 5′ TCGAGAGCTCCACCATGTACGGTGTTCTCCCTTGGAACGCTTTCCCT | 34 |
| | Mutagenic | | | GGC 3′ | |
| 234M | PCR/ | (see 2) | Sac I | 5′ TCGAGAGCTCCACCATGACCTTCCACCTGT 3′ | 35 |
| | Mutagenic | | | | |

All primers were synthesized at the Macromolecular Structure Facility (Michigan State University, East Lansing, MI). Mutated codons are marked in bold. Linker sequences are underlined.
*This primer does not contain an M1 < L mutation, but the removal of 3′ terminal G (vs PLP3) causes polymerase slippage during PCR, generating an ATG < TTG transversion. This effect is not observed with the Pfu Turbo DNA polymerase.
1 Removes all out-of-frame start codons adjacent to M205 and creates a Kozak consensus start site at Met 205
2 Creates a Kozak consensus start site at Met 234

Isolation of Stably Transfected Cell Lines.

G418 Selection Procedure.

All constitutive expression cell lines were isolated by selection with the G418 antibiotic. 24 hrs after cell passage (see above), transfected 293 and NT2 cells were treated with 500 μg/mL G418 (Invitrogen Life Technologies) in DMEM. The selection medium was changed every second day for 2-2.5 weeks, during which the majority of cells detach and G418 resistant colonies emerge. Depending upon the number and density of colonies, surviving cells were grown for 3-5 days in G418-free medium prior to subcloning. Once colonies reached the appropriate size, each plate was briefly examined for fluorescence, and colonies with the lowest number of nonfluorescent, contaminating cells were marked for subcloning.

To isolate marked colonies, the medium was removed and flame sterilized cloning rings were placed around the colonies with a light coating of grease. The cloning rings were filled with trypsin-EDTA (Invitrogen Life Technologies) and the medium on the plate was carefully replaced to prevent dehydration of the nonselected colonies. After a short incubation (˜1 min, RT), trypsin-EDTA treated cells were passed into six-well trays. The cloning rings were removed and the remaining cells grown for pool samples. Both the subclones and the pool plates were fed 24 hrs later. Subclones were grown to ˜80% confluence, then passed for slide preparation and into 60 mm×15 mm stock plates (1:4 ratio). At 50% confluence, the coverslips were mounted on slides . Fluorescence microscopy was then used to assess cell phenotype and the proportion of fluorescent cells. Only subclones with >70% fluorescent cells and pooled samples were retained and frozen for storage. For freezing, cells were grown to 90% confluence, treated with trypsin-EDTA (1 min, RT), collected in 1.5 mL freezing medium (90% fetal bovine serum, 10% DMSO), and transferred to cryotubes. Cryotubes were placed on dry ice for 1 hr to slow freeze cells, then submerged in liquid N₂ for long-term storage.

When transfection efficiency was high, the number of colonies recovered on a selection plate may be too dense for subcloning. In this case, cells were diluted and replated for subcloning. Selection plates were grown in G418-free medium to ˜60% confluence, pooled cells were collected in 10 mL medium, and 1 ml was diluted using 1:2500, 1:3000, 1:3500, and 1:4000 ratios. These dilutions were then passed onto 100 mm×20 mm dishes and allowed to produce colonies. The remaining 9 mL portion was used to prepare a pool sample and frozen at 90% confluence. The dilution plates were grown until colonies were observed (˜1 week), then processed as described for the original selection plates.

Fluorescent Staining of Cellular Structures.

Various cellular compartments of cells expressing the proteolipid proteins were visualized using organelle-specific fluorescent stains. Organelle-specific staining was then compared to the EGFP fluorescence pattern for colocalization analysis.

BODIPY® TR Ceramide.

To visualize the Golgi complex, cells grown on coverslips were rinsed in 1× PBS and fixed in 4% paraformaldehyde (10 min, RT). Fixed cells were rinsed in 1× PBS (twice) and treated with 500 nM BODIPY® TR ceramide in 3% BSA (3% bovine serum albumin in 1× PBS-T) (1 hr, RT). Following incubation, coverslips were rinsed in 1× PBS (three times) and mounted for fluorescent microscopy.

BODIPY® TR-X Phallacidi.

To visualize actin filaments, cells grown on coverslips were rinsed in 1× PBS and fixed in 4% paraformaldehyde (10 min, RT). Fixed cells were washed in 1× PBS (twice) and pretreated with 0.1% Triton X-100 (5 min, RT). These samples were then rinsed in 1× PBS (twice) and incubated with 3% BSA (20 min, RT), prior to addition 165 nM BODIPY® TR-X phallacidin (dissolved in methanol and directly added to 3% BSA). Cells were stained for 20 min at RT, washed in 1× PBS (twice), and the coverslips were mounted for fluorescent microscopy.

LysoTracker® Red DND-99.

To visualize lysosomes, cells grown on coverslips were treated with the 75 nM LysoTracker® Red DND-99 in DMEM (1 hr 15 min; 37° C.). Upon stain removal, cells were rinsed in 1× PBS, fixed in 4% paraformaldehyde (10 min, RT), and rinsed in 1× PBS (three times). The coverslips were then mounted on slides and examined by fluorescent microscopy.

Immunocytochemistry

A comprehensive list of the antibodies used in this work is described in Table 3.

Visualizing PLP/DM20 Proteins in Cells

Transfected cells were stained with the “Nokes” anti-PLP antibody which binds amino acids 269-276 at the C-terminus of PLP/DM20 (Benjamins et al., 1994). Coverslips were rinsed in 1× PBS-T and fixed in 4% paraformnaldehyde (12 min, RT). Fixed cells were then rinsed in 1× PBS-T (twice), permeabilized in 100% methanol (2 min, RT), rinsed in 1× PBS-T (twice), and blocked in 3% BSA (5 min RT). Following two rinses in 1× PBS-T, cells were treated with the Nokes antibody (diluted 1:100, 1 hr, RT). Excess antibody was removed by two rinses in 1× PBS-T. Slides were treated with 3% BSA (5 min, RT) and two rinses in 1× PBS-T. Cells were then stained with goat anti-rabbit FITC (fluorescein isothiocyanate) conjugated IgG (diluted 1:100, 5 min, RT, in the dark). The coverslips were washed in 1× PBS-T (twice), mounted, and examined by fluorescent microscopy.

To visualize the JPLP and JDM20 proteins, pJPLP and pJDM20 transfected cells were stained with the “Morris” anti-Jimpy antibody, which binds to amino acids 235-242 at the C-terminus of the JPLP and JDM20 proteins [241]. The antibody detection procedure was as described above. The primary antibody (anti-Jimpy) was diluted 1:50, while the secondary antibody (goat anti-rabbit FITC conjugate) was diluted 1:100.

TABLE 3
Summary of Antibodies Used

| Antibody | Species | Source | Application |
|---|---|---|---|
| Anti-GFP | rabbit | Molecular Probes; Eugene, OR | Western blotting, 1°° |
| Anti-GFP | rabbit | Clontech; Palo Alto, CA | Immunocytochemistry, 1° |
| Anti-DsRed | rabbit | Clontech; Palo Alto, CA | Western blotting, 1° |
| Nokes(anti- | rabbit | Dr. Skoff, Wayne State University Detroit, MI | Immunocytochemistry, 1° |
| PLP/DM20) | | | |
| Morris(anti-Jimpy) | rabbit | Dr. Skoff, Wayne State University Detroit, MI | Immunocytochemistry, 1° |
| O1° 1 | mouse | Dr. Nave, Max-Planck Institute of Experimental | Immunocytochemistry, 1° |
| (anti-PLP/DM20) | | Medicine, Gottingen, Germany | |
| Anti-PARP (Poly(ADP- | mouse | BIOMOL Research Laboratories; Plymouth | Western blotting, 1° |
| ribose) polymerase) | | Meeting, PA | |
| Anti-Ubiquitin | rabbit | Chemicon; Temecula, CA | Western blotting, 1° |
| Anti-Actin | rabbit | Sigma-Aldrich; St Louis, MO | Immunocytochemistry, 1° |
| Anti-BiP | mouse | StressGen Biotechnologies; Victoria, BC | Immunocytochemistry, 1° |
| Anti-SP1° | rabbit | Chemicon; Temecula, CA | Immunocytochemistry, 1° |
| Golgi Zone | mouse | Chemicon; Temecula, CA | Immunocytochemistry, 1° |
| Anti-Rabbit-HRP | goat | Amersham Pharmacia Biotech, UK | Western blotting, 2° |
| conjugate | | | |
| Anti-Mouse-HRP | goat | Amersham Pharmacia Biotech, UK | Western blotting, 2° |
| conjugate | | | |
| Anti-Rabbit-FITC | goat | Kirkegaard & Perry; Gaithersburg, MD | Immunocytochemistry, 2° |
| conjugate | | | |
| Anti-Rabbit- | goat | Kirkegaard & Perry; Gaithersburg, MD | Immunocytochemistry, 2° |
| Rhodamine conjugate | | | |
| Anti-Mouse- | goat | Kirkegaard & Perry; Gaithersburg, MD | Immunocytochemistry, 2° |
| Rhodamine conjugate | | | |
| Anti-Mouse-TRITC | goat | Kirkegaard & Perry; Gaithersburg, MD | Immunocytochemistry, 2° |
| conjugate | | | |

To examine folding and transport of the fluorescent fusion proteins in Δ2 EGFP and Δ6 EGFP transfectants, cells were stained with the undiluted supernatant from cultured O10 hybridoma cells. The O10 monoclonal antibody (mAb) recognizes an extracellular epitope in the wt PLP/DM20 proteins which is conformation sensitive [288]. Slides were rinsed in 1× PBS, incubated with undiluted O 10 supernatant for 45 min at RT, rinsed in 1× PBS, and fixed in 4% paraformaldehyde (I 5 min; RT). The samples were then washed in 1× PBS (5 min; RT) and stained with goat anti-mouse TRITC (tetramethylrhodamine isothiocyanate) conjugated IgM (diluted 1:300, 30 min, RT). The coverslips were washed in 1× PBS (5 min, RT) and mounted for fluorescent microscopy.

EGFP fluorescence decreases in cells expressing mutant PLPs. To ensure that the decline in EGFP fluorescence was not due to chromophore misfolding or destabilization in a mutant protein, JPLP-EGFP and JDM20-EGFP transfected cells were stained with an anti-GFP antibody (Clontech). The procedure was as described for the “Nokes” anti-PLP staining procedure. The primary antibody (anti-GFP) was diluted 1:1000, while the secondary antibody (goat anti-rabbit rhodamine conjugated IgG) was diluted 1:250. Following the final washes in 1× PBS-T, coverslips were either mounted for fluorescence microscopy or stained with DAPI prior to mounting.

Antibody Detection of Subcellular Structures

As an alternative to organelle-specific fluorescent dyes, organelle-specific antibodies were also used to colocalize the PLP-EGFP and DM20-EGFP proteins. For anti-SP1 and anti-Actin primary antibodies, the secondary antibody was the same as in the anti-GFP procedure. For anti-BiP and Golgi Zone primary antibodies, the secondary antibody was goat anti-mouse rhodamine conjugated IgG.

Fluorescent Microscopy and Photography.

Live cells and slides were examined by fluorescent microscopy using a Leica DM IRB inverted research microscope (Leica Microsystems, Wetzlar, Germany). Cells expressing fluorescent fusion proteins were visualized with the following filters: HQ:GFP filter (EGFP cells), HQ:Yellow GFP filter (EYFP cells), Cyan GFP filter (ECFP cells) and HQ:TRITC (DsRed cells). PI and DAPI stained slides were examined with HQ:Texas Red and DAPI/Hoechst/AMCA filters, respectively. BODIPY® TR ceramide, BODIPY® TR-X phallacidin, and LysoTracker® Red DND-99 stained slides, as well as rhodamine or TRITC labeled immunocytochemistry slides, were examined with the HQ:TRITC filter. FITC labeled immunocytochemistry slides were examined with a FITC filter. Colocalization of EGFP fusion proteins and fluorescent stains or antibody conjugates was visualized with a Triple DAPI/FITC/TRITC filter (which allows the simultaneous detection of red, green, and blue fluorescence). All filters were purchased from Chroma Technology Corporation (Brattleboro, Vt.).

TABLE 4
Visualization Methods for Cellular Structures

| Cellular | Fluorescent Staining | | Immunocytochemistry | | |
|---|---|---|---|---|---|
| Structure | Stain | Color | 1° Ab | 2° Ab | Color |
| Nucleus | DAPI | Blue | Anti-SP1 | Anti-rabbit, rhodamine | red |
| | | | 1:100 | conjugate 1:100-1:250 | |
| Endoplasmic | — | — | Anti-BiP | Anti-mouse, rhodamine | red |
| Reticulum (ER) | | | 1:100 | conjugate 1:100-1:250 | |
| Golgi Complex | BODIPY ® TR | red | Golgi Zone | Anti-mouse, rhodamine | red |
| | ceramide | | 1:100 | conjugate 1:100-1:250 | |
| Actin Filaments | BODIPY ® TR-X | red | Anti-Actin | Anti-rabbit, rhodamine | red |
| | phallacidin | | 1:100 | conjugate 1:100-1:250 | |
| Lysosomes | LysoTracker ® Red | red | — | — | — |
| | DND-99 | | | | |

Protein Preparation and Western Blotting.

Cell Harvesting and Protein Extraction.

Cells were grown in 100 mm or 60 mm dishes to ˜80% confluence, then harvested in native medium. The cells were pelleted to remove medium (5 min, 800 rpm, RT), washed in 1 mL 1× PBS, and centrifuged (1 min, 6K, RT). The pellets were often stored at −70° C. For protein extraction, cells were thawed on ice and resuspended in equal volumes of Protein Extract Suspension Buffer (100 mM NaCl, 10 mM Tris-HCl [pH 7.6], 1 mM EDTA, 1 mg/mL aprotinin, 100 μg/mL PMSF) and 2× SDS Buffer (100 mM Tris-HCl [pH 6.8], 4% SDS, 20% glycerol, 200 mM DTT). The extracts were homogenized by several passages through a 1 mL syringe fitted with a 26 G needle. Samples were then incubated at RT for 1 hr. Samples were then frozen at −70° C. for storage until SDS PAGE and Western blot analysis. It was vital that samples were not heated above 37° C., since the PLP/DM20 proteins aggregate at high temperatures.

SDS PAGE and Western Blotting

SDS PAGE gels were prepared with OmniPur Pro-Gel® gel concentrates and buffers [6%, 8%, 10%, and 12%] (EM Science) following the commercial procedure. To insure equal sample loading on Western blots, 10 μL of each protein extract was resolved on 6% gels, fixed overnight in Preblot gel fixer (25% isopropanol, 10% acetic acid), and stained in 0.05% Coomassie blue (0.05% brilliant blue R, 50% methanol, 10% acetic acid) (20 min, RT). Gels were destained in 10% acetic acid (2-4 hrs, RT) and dried. Based on protein staining in each lane, any necessary volume adjustments were made to each sample prior to Western blotting. In general, optimal resolution of the PLP, DM20, JPLP, and JDM20 fluorescent fusion proteins was achieved on 8% SDS PAGE gels.

Following SDS PAGE, proteins were transferred onto a nitrocellulose membrane (Amersham) by electrophoresis (1 hr, 100 V, 4° C.) using a vertical transfer system (Hoeffer) and the membranes were dried overnight. The membranes were rehydrated in 1× PBS-T (4×5 min) and blocked with 5% milk powder dissolved in 1× PBS-T (4×15 min, RT). The milk solution was removed with 1× PBS-T (3×5 min, RT). Prior to antibody addition, the membranes were washed in 3% BSA (5 min, RT). The samples were then incubated with an anti-GFP antibody (Molecular Probes) diluted 1:1000-1:1200 in 3% BSA. Excess antibody was removed with 1× PBS-T (3×5 min, RT) and the membranes washed in 3% BSA (5 min, RT). The membranes were then incubated with horseradish peroxidase (HRP) conjugated goat anti-rabbit IgG (Amersham) for 1 hr, RT [1:5000-1:6500 dilution]. Excess antibody was removed with 1× PBS-T (30 min, RT, then 6×5 min, RT) and detected with the ECL reagent system as described by the manufacturer (Amersham). Individual protein bands were quantitated by densitometry using the Gel Area Scan software on a Beckman DU7400 Spectrophotometer.

Proteasome Inhibition Assays.

Proteasome inhibition assays were carried out in stably transfected cell lines using MG132 (Z-Leu-Leu-Leucinal)—a potent, cell permeable inhibitor of the 26 S proteasomal complex (Calbiochem, San Diego, Calif.). The MG132 concentration in all experiments (50 μM) was sufficient to insure 100% cell death of all 293, NT2, and 293-Tet-On cell lines in 24 hrs.

Visualizationi of Cell Death in MG132 Treated 293 and NT2Cell Lines.

To confirm that 293 and NT2 cultures undergo apoptosis within 24 hrs post treatment with 50 μM MG132, cells were assayed for poly(ADP-ribose) polymerase (PARP) cleavage by caspase-3. The 293 and NT2cultures were grown in 100 mm dishes to ˜90% confluence and passed at equal density into a six well tray. At 70-80% confluence, wells were treated with 50 μM MG132 in DMEM and harvested for proteins at 0.5 hrs, 1 hr, 2 hrs, 4 hrs, 6 hrs, and 24 hrs after treatment. The PARP cleavage products were resolved on 4% SDS PAGE gels. The Western blots were incubated with an anti-PARP (BIOMOL Research Laboratories) primary antibody diluted 1:2000 in 3% BSA, and a HRP conjugated goat anti-mouse secondary antibody (Amersham) diluted 1:2500 in 3% BSA.

Site Directed Mutagenesis

AUG1 (M1L) Start Codon Mutants

M1L/K268R, M1L/K275R, M1L/K268R/K275R, and M1L/jimpy mutants were generated during the construction of the K268R, K275R, K268R/K275R, and jimpy EYFP fusion constructs by PCR mutagenesis. When the MC5 primer (see Table 2) was used with the Qiagen or Invitrogen Taq polymerases to amplify the proteolipid cDNAs, this primer generated an ATG to TTG mutation in the start codon (M1L mutation).

The amplification products were cut with EcoRI and SacII, gel purified and cloned into the EYFP-N1 vector. Ligation products were transformed into LE392 competent cells, selected for kanamycin (Kan) resistance, and sized versus the EYFP vector. To confirm the presence of K268R, K275R, K268R/K275R, and jimpy mutations, positive clones were sequenced.

QuikChange® Production of M1L, M205L, and M234L Mutants.

To insert methionine mutations into other vector sites, the QuikChange® Site-Directed Mutagenesis Kit (Stratagene) was employed. All QuikChange® mutagenic primer sets are described in Table 2. All vector sequences were confirmed by DNA sequence analysis.

Table 5 below lists vectors encoding various wt and mutant PLPs and PIRPs including optimized sequences. Experssion vectors are indicated

TABLE 5
Vector Information Summary.

| Vector | Parental Vector | Mutation/Added Sequences | Protein tag * |
|---|---|---|---|
| PLP-GFP/DM20-GFP | pEGFP-N1/EYFP-N1 | PLP and DM20 cDNAs | g/y GFP |
| PLP-GFP/DM20-GFP Tet-On | pEGFP-Tet-On | PLP and DM20 cDNAs | g GFP |
| PLP-GFP/DM20-GFP M1L | PLP-GFP/DM20-GFP | start codon mutation (M1L) | y GEP |
| PLP-GFP/DM20-GFP M1L/M205L | PLP-GFP/DM20-GFP M1L | M1L and M205L mutations | y GFP |
| PLP-GFP/DM20-GFP M1L/M234L | PLP-GFP/DM20-GFP M1L | M1L and M234L mutations | y GEP |
| PLP-GFP/DM20-GFP M1L/M205L/M234L | PLP-GFP/DM20-GFP M1L/M205L | M1L, M205L and M234L mutations | y GFP |
| PLP-GFP/DM20-GFP Pro- | pEGFP-Tet-On | promoter deletion | g GFP |
| JPLP-GFP/JDM20-GFP | pEGFP-N1/EYFP-N1 | Jp¹ PLP and DM20 cDNA | g/y GFP |
| JPLP-GFP/JDM20-GFP M1L | JPLP-GFP/JDM20-GFP | start codon mutation (M1L) Jp bkgrd | g/y GEP |
| JPLP-GFP/JDM20-GFP M1L/M205L | JPLP-GFP/JDM20-GFP M1L | M1L and M205L in Jp background | g/y GFP |
| RshPLP-GFP/RshDM20-GFP M1L | PLP-GFP/DM20-GFP M1L | M1L and I186T (Rsh²) mutations | y GFP |
| PLP-GFP/DM20-GFP M1L/K268R | PLP-GFP/DM20-GFP M1L | M1L and K268R mutations | y GFP |
| PLP-GFP/DM20-GFP M1L/K275R | PLP-GFP/DM20-GFP M1L | M1L and K275R mutations | y GFP |
| PLP-GFP/DM20-GFP M1L/K268R/K275R | PLP-GFP/DM20-GFP M1L | M1L, K268R, and K275R mutations | y GFP |
| PLP-GFP/DM20-GFP M1L/R272K | PLP-GFP/DM20-GFP M1L | M1L and R272K mutations | y GFP |
| Expression Vector | | (Proteins Expressed) | |
| 205M-CMV/234M-CMV | PCMV | PIRP-M³ /PIRP-L⁴ ORFs only, | None |
| | | out-of-frame start codon deletions | |
| 205M-His-CMV/234M-His-CMV | 205M-CMV/234M-CMV | PIRP-M³ /PIRP-L⁴ ORFs only, | 6XHis |
| | | out-of-frame start codon deletions | |
| 205M-BsKS+/234M-BsKS+ | PBluescript II KS+ | PIRP-M³ /PIRP-L⁴ ORFs only, | none |
| | | out-of-frame start codon deletions | |
| 205M-His-BsKS+/ 234M-His-BsKS+ | PBluescript II KS+ | PIRP-M³ /PIRP-L⁴ ORFs only, | 6XHis |
| | | out-of-frame start codon deletions | |
| 205M-ET-14b/234M-ET-14b | pET-14b | PIRP-M³ /PIRP-L⁴ ORFs only, | 6XHis |
| | | out-of-frame start codon deletions | |

¹Jp = murine jimpy mutation (exon 5 deletion and frameshift in exons 6 and 7)
²Rsh = murine rumpshaker mutation (I186T)
³PIRP-M = PLP IRES Protein M, M205 initiation product
⁴PIRP-L = PLP IRES Protein L, M234 initiation product
* g = Green y = yellow GFP-green fluorescent protein

### EXAMPLE II

### Discovery of Novel Protein Isoforms Synthesized from the PLP and DM20 mRNA Transcripts During Apoptosis

In addition to their structural role in myelin, the PLP and DM20 proteins exhibit growth factor activity, participate in cell-cell and cell-ECM communications, and regulate the survival and differentiation of OL progenitors, OLs, astrocytes, and neurons. The present inventors developed a cell based expression system to examine the synthesis, transport and turnover of the PLPs. This system detected previously unknown translational events in the PLP and DM20 transcripts which appear to be produced by IRES translational regulation. As previously described, cellular genes containing IRES sequences encode an elite group of proteins that regulate cell growth, differentiation, survival, and apoptotic death. Therefore, it seems apparent that this important type of translational regulation is significant for the myelin proteolipid protein gene.

Expression of PLP and DM20 cDNA Constructs Tagged with the GFP

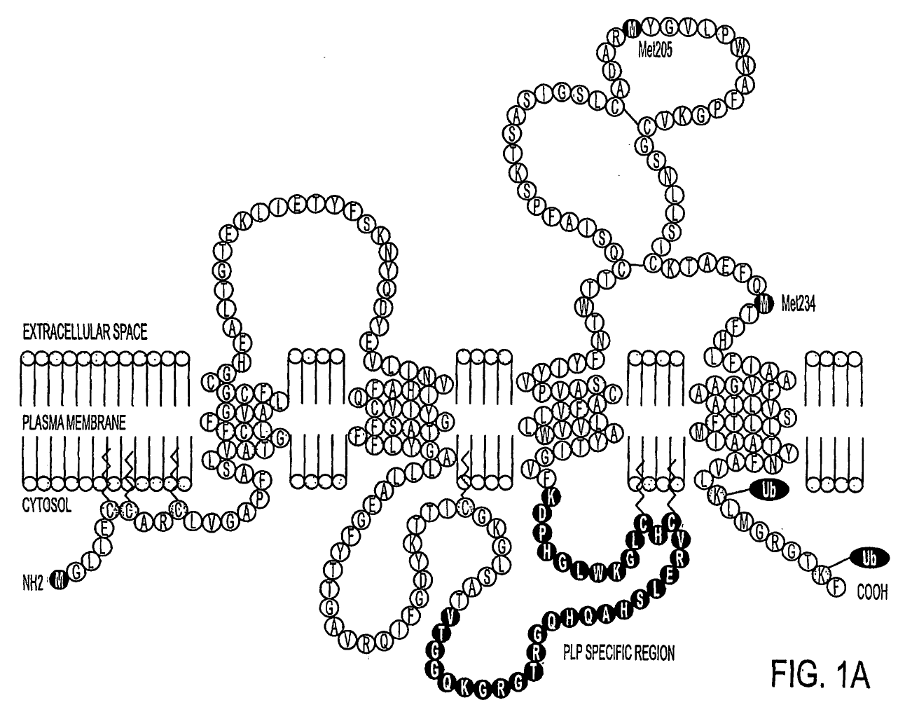

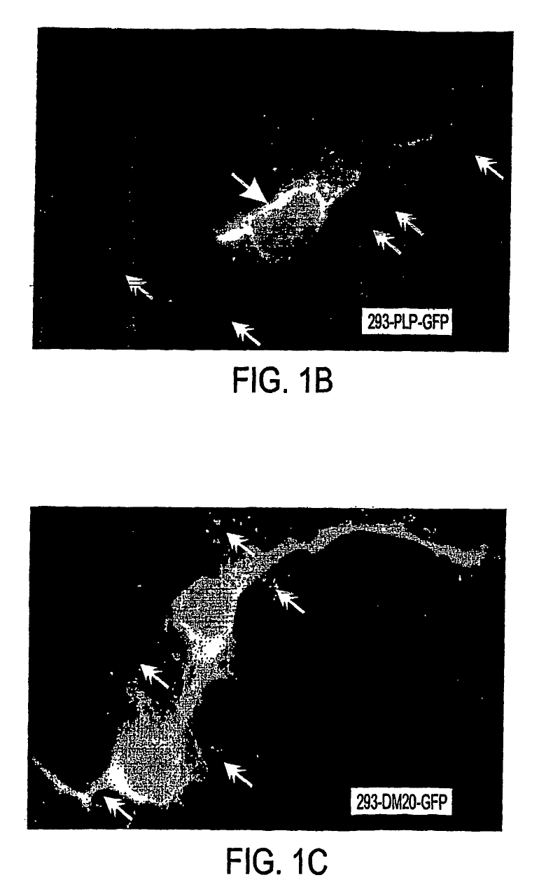

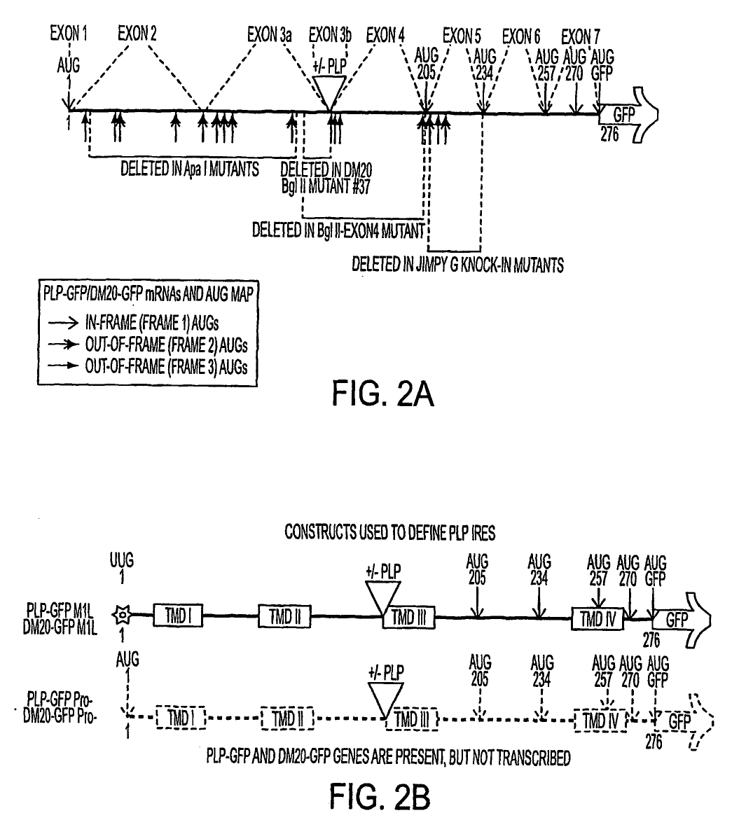

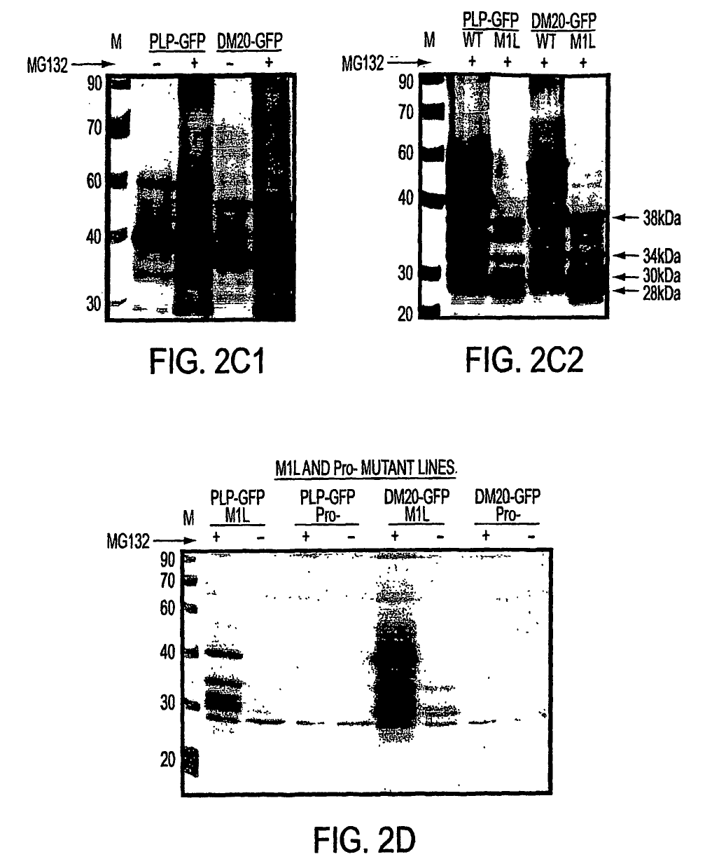

Two cytomegalovirus (CMV) promoter vectors were prepared which express the PLP and DM20 cDNAs as either native proteins (the pCMV vector) or as a fusion protein with the GFP (pEGFP plasmid) (FIG. 2A). Since the C-terminus of the PLP/DM20 protein is a charged, cytosolic sequence, it seemed likely that fusing the soluble EGFP protein to this sequence would not significantly alter the structure of the PLP. A variety of immunostaining assays using anti-PLP antibodies detected no differences in the synthesis or transport of the native or fluorescent fusion proteins in transiently or stably transfected 293 (human embryonic kidney) and NT2 (human teratocarcinoma) cells (FIG. 1B).

Initial Evidence of Internal Translation Initiation

Western blot analysis of normal and mutant PLP/DM20 fusion protein samples consistently revealed the presence of LMW (Low Molecular Weight) proteins in the 25-40 kDa range. Treating cells with MG132 resulted in a dramatic increase in the concentration of several of these LMW proteins. Studies were performed to show that one or more of these LMW species were distinct from the C-terminal PLP/DM20 fragment shown to act as a secreted regulator of oligodendrocytic maturation and survival—for which little was known about the size or the mechanism for its generation. Because the present inventors' protease inhibitor studies failed to identify a protease responsible for generating the LMW peptides from the full length PLP/DM20 proteins, the present inventors predicted, and then discovered, that these peptides were produced by internal translation initiation towards the 3′ end of the PLP/DM20 ORF.

Met¹ Mutant Phenotypes

During construction of external K→R mutant plasmids by PCR based mutagenesis, a point mutation (ATG→TTG) was introduced into the native PLP/DM20 start codon of the Δ2 EYFP and Δ6 EYFP templates. As a result, the (−AUG) PLP-EYFP M1L/K268R, PLP-EYFP M1L/K275R, PLP-EYFP M1L/K268R/K275R, DM20-EYFP M1L/K268^{Plp}R, DM20-EYFP M1L/K275^{Plp}R, and DM20-EYFP M1L/K268^{Plp}R/K275^{Plp}R plasmids were recovered.

All of the (−AUG) external K→R mutant plasmids were transfected into 293, NT2, and Cos-7 cells. Slides were prepared every 24 hrs from Day3 to Day8 post transfection and stained with DAPI. Since the UUG codon is rarely employed for translation initiation (Williams et al., 2001), normal mRNA translation was not expected from the PLP-EYFP M1L/K268R, PLP-EYFP M1L/K275R, PLP-EYFP M1L/K268R/K275R, DM20-EYFP M1L/K268^{Plp}R, DM20-EYFP M1L/K275^{Plp}R, and DM20-EYFP M1L/K268^{Plp}R/K275^{Plp}R vectors. However, every slide contained EYFP positive cells. The majority of transfected cells displayed very low levels of the fluorescent fusion protein (i.e., dim cell bodies). High fluorescence levels were associated with cell death at all time points. In dim cells, EYFP fluorescence was excluded from the nucleus arid appeared to associate with the membranes of the proximal secretory compartments, such as the endoplasmic reticulum and cis-Golgi complex (ER/cis-GC). None of the fusion protein appeared to reach the cell surface, as judged by the absence of the microvilli labeling. In moderate to bright cells, the fusion protein often formed bright aggregates in the perinuclear region, which persisted in dead and dying cells.

To determine whether protein synthesis was initiated from inside the PLP-EYFP and DM20-EYFP ORF's, transfected 293 pooled cultures were prepared by G418 selection. These pools were collected and treated with MG132 for 24 hrs, then harvested and the proteins prepared as described. Western blot analyses using an anti-GFP antibody found that MG132 treatment of cells expressing the wildtype or M1L mutant transcripts produced a ladder of indistinguishable low molecular weight (LMW) proteins (˜38 kDa, ˜34 kDa, ˜30 kDa and ˜28 kDa) (FIG. 2D and Table 5). No full length PLP or DM20 proteins were detected in M1L samples. Since the molecular weight of the EGFP protein is ˜27 Da, the PLP/DM20 C-terminal peptide in the LMW proteins would range from 1-11 kDa (Table 6).

TABLE 6

| Putative Internal | | Calculated | |
|---|---|---|---|
| Translation Initiation | Calculated length | Size | Apparent Size, |
| Codons | (aa) | (kDa) | kDa (Western) |
| Met²⁰⁵ | 322 | 37.3 | ˜38 |
| Met²³⁴ | 293 | 33.6 | ˜34 |
| Met²⁵⁷ | 270 | 30.8 | ˜32 |
| Met²⁷⁰ | 257 | 29.1 | ˜31 |
| EYFP Met¹ | 240 | 27.0 | ˜30.5 (very faint) |

Since cells expressing the wildtype PLP/DM20 proteins contained both the full-length and LMW proteins, the cap-dependent initiation codon and internal open reading frames did not eliminate synthesis of the C-terminal proteins (FIG. 3B&C). Furthermore, detection of the LMW proteins in M1L mutants eliminates any possibility of proteolytic processing of the full-length protein to generate the LMW proteins. Alternative explanations for these observations include;
(1) The LMW proteins could be produced by cryptic splicing in the proteolipid cDNAs. If one splicing event produces the LMW proteins, then Met¹ and each intervening ORF must be removed. When constructing the proteolipid expression vectors, virtually all of the PLP/DM20 5′ untranslated sequences were removed to eliminate residual transcriptional regulatory elements and translational inhibitor sequences (retaining only 10 bp upstream of the AUG codon). Since the LMW proteins are synthesized from the native cDNAs (FIGS. 2 and 3), Met¹ would be removed by splicing within this 10 bp or within the CMV promoter. Since cryptic splice sites are generally larger than 10 bp and no splicing activity has been detected from the CMV promoter, it seems unlikely that a single splicing event could produce the LMW proteins. A second possibility is that several internal splicing events generate multiple transcripts which initiate translation from Met¹. However, RT PCR analysis using primers which encompass Met¹ and the proteolipid stop codon did not detect any internally spliced RNA products. Therefore, it appears that cDNA splicing cannot generate the C-terminal LMW proteolipid proteins.
(2) The LMW proteins could be produced by a cryptic promoter in the PLP and DM20 cDNA sequences. This promoter would map close to the carboxyl terminus, respond to apoptotic stress and synthesize truncated mRNA species. However, deletion of the CMV promoter eliminated LMW protein synthesis during apoptosis (see below). This observation is inconsistent with the hypothesis that the LMW proteins are derived from a cDNA-specific transcriptional activity.
(3) The LMW proteins could be produced by translation from internal initiation codons (see below). All of the evidence is consistent with the conclusion that the LMW proteolipid proteins are synthesized by internal translation activity regulated by a proteolipid IRES element.
   The Proteolipid cDNA Does Not Contain a Cryptic Promoter.

Standard recombinant techniques were used to delete the CMV promoter from the PLP and DM20 expression vectors and the new vectors were named PLP-GFP Pro- and DM20-GFP Pro-. Stable cell lines were examined for steady state and apoptotic induction of LMW protein synthesis (FIG. 2B-2E). Western blot analysis did not detect any protein synthesis which indicated that the LMW proteolipid proteins were not generated from a cryptic cDNA promoter.

IRES Structural and Sequence Elements in the Proteolipid Transcripts

During cap-dependent translation, the majority of eukaryotic mRNAs select a start codon using interactions between the 5′ mRNA cap and the preinitiation complex, followed by ribosome scanning to the first Met codon. In the PLP/DM20 M1L transcript, ribosome scanning should initiate translation at the next downstream AUG codon which would be out-of-frame with the EGFP moiety. In fact, sequence analysis detected a number of open reading frames (ORFs) between Met¹ and any C-terminal initiation codon which would be in-frame with the EGFP gene (i.e., Met²⁰⁵ or Met²³⁴ in the PLP sequence) (FIG. 2A). These upstream ORFs prevent ribosome scanning to Met²⁰⁵/Met²³⁴ and indicate that translation initiation from these codons must employ an alternative system, such as cap-independent translation.

Cap-independent translation requires a cis -acting RNA sequence termed an “internal ribosome entry site” or IRES. Although IRES regulatory sequences can functionally substitute for one or more components of the cap-dependent translation initiation machinery, any given IRES element shares little primary sequence identity to any other IRES sequence. Nonetheless, IRES elements tend to form stable secondary structures and possess one or more of the following sequences: (1)18S rRNA homology or complementarity regions; (2) polypyrimidine tracts; (3) GNRA elements, where N is any nucleotide and R is a purine and (4) AGACA sequences (see Background). Although not well defined, many of these IRES sequences apparently bind translational effector proteins which are required for IRES function.

Examination of the PLP/DM20 mRNAs for “IRES-like” sequence or structures revealed the following:
(a) The capacity to form a stable stem-loop RNA secondary structure immediately upstream of the internal, in-frame AUG codons (Met²⁰⁵ and Met²³⁴) which is similar to the stem-loop A structure of picornaviruses.
(b) An 18S rRNA complementarity region located 90 nucleotides upstream of Met 205 in exon 3a which is highly similar to a related sequence in the Gtx IRES.
(c) Multiple polypyrimidine tracts (PPT) and GNRA elements scattered throughout the proteolipid gene upstream of the Met²⁰⁵ and Met²³⁴ codons. The PLP transcript contains 17 PPTs (5-18 nucleotides long and 13 GNRA elements, while the DM20 mRNA contains 16 PPTs and 11 GNRA elements.
(d) Significant evolutionary conservation of the proteolipid gene sequence encompassing the putative IRES element. Alignment of the mouse, rat, human, dog, rabbit, pig, cow, chicken, Chinese turtle, and frog gene sequences between the 18S complementarity region and Met²⁰⁵ proved that these sequences were 87-97% identical. Furthermore, Met²⁰⁵ and Met²³⁴ were present in higher vertebrate genomes.

Therefore, strong sequence conservation, even in the codon wobble position of these divergent species, suggest that this segment of the proteolipid transcript contains a functional element which is most likely the IRES sequence.

Assignment of Internal Translation Initiation Codons to PLP/DM20 IRES Proteins

Since none of the alternative translation initiation codons were found in-frame with the EGFP protein (i.e., ACG, AUU, CUG, or GUG), the LMW proteins appeared to initiate translation from ˜Met²⁰⁵ and ˜Met²³⁴. Site directed mutagenesis was used to alter the Met¹, Met²⁰⁵ and Met²³⁴ codons to UUG and LMW protein expression examined in stable cell lines treated with MG132 (FIG. 3A-3C). As before, MG132 treatment of M1L mRNA expressing cells induced the expression of a complete set of LMW proteins ˜38 kDa, ˜34 kDa, ˜30 kDa and ˜28 kDa. However, further study found that some of these proteins were resistant to SDS denaturation and produced a condensed protein structure which migrated faster than its calculated molecular weight on SDS-PAGE gels. Generally, these SDS resistant proteins (i.e., the 30 kDa Met²⁰⁵-specific and 28 kDa Met²³⁴-specific proteins) aggregated after samples were heated above 55° C. and did not enter the gels. Therefore, the loss of the 38 kDa and 30 kDa proteins in M1L/M205L samples reflect the translation of the 38 kDa protein species from Met²⁰⁵. Similarly, the loss of 34 kDa and 28 kDa proteins in the M1L/M234L sample correlates with translation of the 34 kDa protein species from Met²³⁴. These results strongly suggest that two internal translation sites are utilized during cap-independent translation of the proteolipid transcript and that 10.3 kDa and 6.6 kDa proteins (actual sizes calculated from protein sequence) are produced from the Met²⁰⁵ and Met²³⁴ codons, respectively. The ˜10 kDa protein has been named the proteolipid IRES protein M (PIRP-M) and the ˜7 kDa protein termed the proteolipid IRES protein L (PIRP-L).

Regulation of Proteolipid IRES Translation

Initially, the synthesis of a 30 kDa protein from the M1L/M205L/M234L plasmid appeared puzzling to the inventors. These mutations should have removed all of the relevant initiation codons and eliminated IRES-mediated protein production. However, the lack of heat sensitivity indicated that the 30 kDa protein is not an SDS-resistant form of the PIRP-M or PIRP-L protein and suggests that this protein is produced by ribosome scanning to downstream Met²⁵⁷, Met²⁷⁰ or Met^{GFP} codons (FIG. 3A).

Support for this proposal was provided by the observation that mRNAs containing the Met234 codon exhibit little 30 kDa protein. Furthermore, Western analysis found that the PIRP-L protein, translated from Met²³⁴, is expressed in steady state cells. Similarly, cells expressing a M1L/M234L transcript now synthesized the 30 kDa product during steady state conditions. These results suggested that ribosome binding occurs upstream of Met²³⁴ and produces steady state synthesis of the PIRP-L protein, but when Met²³⁴ is absent, ribosomes scan to the next available initiation codon.

It was also noticed that preferential PIRP protein synthesis was detected in cells expressing a DM20 transcript. When PLP and DM20 samples were analyzed in adjacent gel lanes, PIRP protein synthesis from the DM20 cDNA was invariably 25-50% higher than PLP levels (FIGS. 2C-2E and 3B-C).

This suggested that PLP-specific sequences or structures alter IRES activity and result in higher protein synthesis from the DM20 mRNA. Preferential translation might affect PIRP protein expression during development, a time when the DM20 mRNA is preferentially expressed.

Internal Translation Initiation Events in the Presence of the Full Length PLP and DM20 Fusion Proteins

The studies exemplified thus far only examined cap-independent stress-induced translation of the PIRP/DM20 proteins in the absence of the native cap-dependent initiation codon (i.e., the Met¹ AUG-mutants). It had not been proved that the LMW proteins could be synthesized from the wt proteolipid mRNA. Although correlative Western blots shown in Chapters 1 and 2 contain LMW proteins, a side-by-side comparison had not been performed.

To compare stress induced translation from the PLP/DM20 IRES in Met¹ AUG+ and Met¹AUG⁻ cells; PLP-EYFP, PLP-EYFP M1L, DM20-EYFP, and DM20-EYFP M1L expressing cell lines were treated with MG132 and protein samples were examined using Western blot analysis (see FIG. 2C). The M1L (−AUG) samples treated with MG132 generated LMW proteins that were indistinguishable from the wt LMW proteins present in the PLP-EYFP and DM20-EYFP samples. Moreover, since both Met¹⁻ and Met¹⁺ samples initiated translation at internal sites in response to MG132 treatment, the mere presence of the cap-dependent initiation codon on the native mRNA did not prevent cap-independent translation of the LMW species during cellular stress.

It was concluded that internal translation initiation occurs from the (biologically relevant) native PLP/DM20 mRNA structure and that this translation is modulated by cellular stress. These findings further support the biological significance of the PLP/DM20 IRES element and the role of PIRP proteins (particularly as growth factors) following IRES activation.

### EXAMPLE III

### Synthesis of a Novel PIRP Protein from the Jp PLP/DM20 Gene

As described above, the severe jp mutation introduces a gain of function phenotype into affected animals which cannot be overcome by gene replacement technology. It has been suggested that this mutation interferes with developmental processes through signal transduction systems in developing OLs. Therefore, it was of interest to determine if the jp mutation which alters the PIRP-M protein sequence inactivates the proteolipid IRES.

In contrast to jimpy animals, animals with the milder rumpshaker (rsh) mutation, which maps to exon 4 and does not directly affect the PIRP proteins, exhibit no obvious developmental deficits. Therefore, a mutant PIRP protein contributes to the distinct developmental defect observed in jp animals which is not evident in rsh mutants.

Since the jp splicing mutation removes exon 5 and causes a frameshift in the PIRP sequences in exons 6 and 7, the present inventors predicted that the PIRP-M protein should be replaced with a Cys-rich 7 kDa peptide (the PIRP-J protein, SEQ ID NO:18, encoded by the nucleic acid having the sequence SEQ ID NO:17.

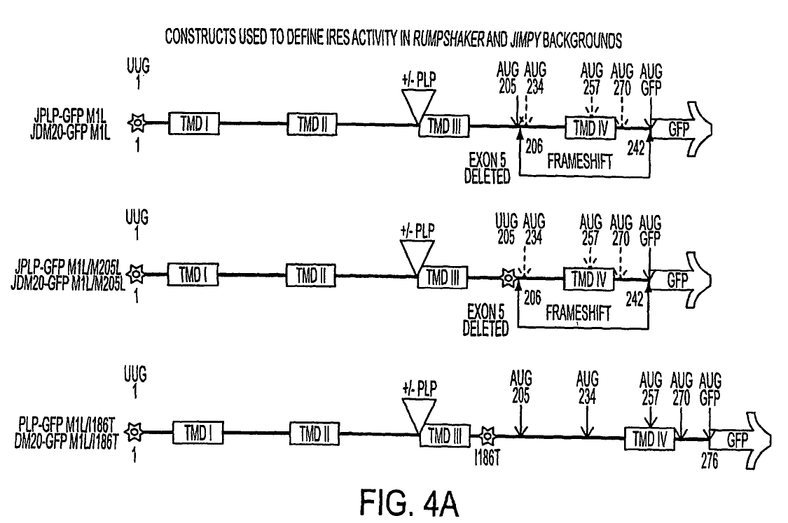

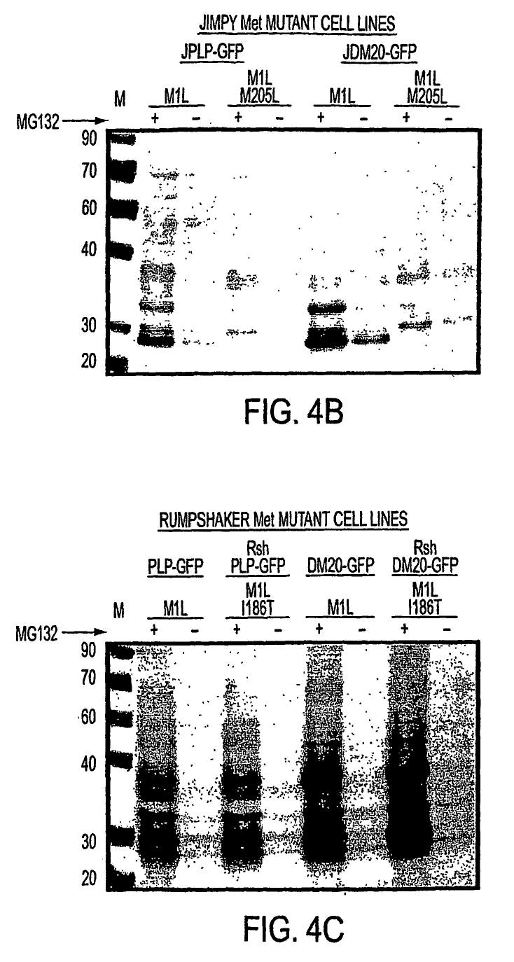

In contrast, the Met²³⁴ ORF which initiates in exon 6 is unaffected by the jp mutation; however, the position of the Met²³⁴ codon is shifted severely within the IRES structure and any control sequences or ribosome binding sites between Met²⁰⁵ and Met²³⁴ would be deleted. In this initial study, expression of the PIRP-J protein from the jp PLP and DM20 transcripts was verified (FIG. 4A-C). Western analysis showed that MG132 treatment of cells expressing the jp PLP/DM20 M1L transcript synthesized a novel set of LMW proteins of ˜34, ˜30 and ˜28 kDa. In contrast, the jp M1L/M205L transcript expressed only minimal amounts of a 30 kDa protein.

It was concluded that the 34 kDa and 28 kDa PIRP-J proteins are translated from Met²⁰⁵ and the 30 kDa protein species is derived from Met^{GFP} via ribosome scanning. The presence of two Met²⁰⁵ protein species suggest that the Cys-rich PIRP-J protein is partially denatured by SDS and migrates unusually fast on SDS-PAGE gels similar to the PIRP-M/L proteins. While it has not been proved that the Met234 codon is blocking ribosome scanning, the extremely low level of 30 kDa MetGFP protein is consistent with this mechanism. In any case, these studies verify that a jp-specific PIRP protein is synthesized from the jp PLP and DM20 transcripts.

In contrast, cells expressing the M1L/I186T rsh transcript did not exhibit any change in the expression levels of the PIRP-M or PIRP-L proteins or in the the size of the product. Therefore, as verified in the 293 cell line expression system, the rsh mutation does not significantly lower IRES activity or inhibit PIRP production.

### EXAMPLE IV

### Expression of PIRP Proteins and PIRP-Expressing Cell Lines

Construction of PIRP Expression Vectors

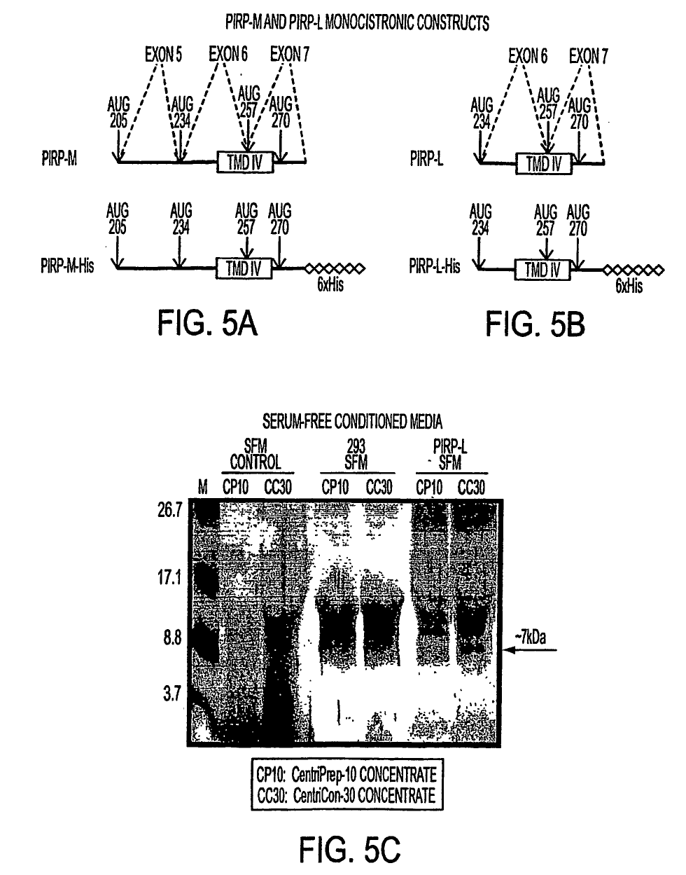

Since PIRP expression is regulated by apoptotic induction of the proteolipid IRES, a variety of long term cellular studies were not possible. To overcome this limitation, the PIRP-M and PIRP-L cDNAs were subcloned into the pCMV vector, so that protein synthesis was regulated by the CMV promoter (FIG. 5A). Although the PIRP-M coding sequence was not altered, several out of frame AUG codons flanking Met²⁰⁵ were removed. In addition, the sequences flanking Met²⁰⁵ and Met²³⁴ were mutated to match a “Kozak” consensus start site [128]. These protein and nucleic acid sequence are provided above (SEQ ID NO:9-16, including ths His-tagged constructs). The vectors are summarized in Table V in Example I.

These changes optimized the PIRP gene cassette for expression in mammalian cells. After transfection, these optimized expression vectors provided convincing evidence of PIRP-assocaited growth factor activity as discussed below.

Immunodetection of PIRPs

Detection of the small PIRPs has not been simple. In addition to their tendency to pass through membranes during electroblotting, these proteins contain a limited number of charged epitopes. This has limited immunological detection to a single commercial monoclonal antibody which reacts with the proteolipid C-terminus, the epitope of which appears to depend on the presence of the terminal Phe residue [192]. The effectiveness of this antibody was limited by the unusual hybridization procedure required to detect this epitope (long incubations at low temperatures). Preliminary studies with this antibody did not detect the recombinant PARP proteins on Western blots, even though their biological activity was apparent. This may be a technical issue (epitope inaccessibility on Western blots) may be related to protein processing that removes the C-terminal epitope or even the one Phe residue. This issue is addressed by the preparation of additional anti-PLP antibodies.

Bacterial Clones

To complement the mammalian expression system, the PIRP cDNAs were also cloned into the bacterial pBSII vector. This DNA functions as a template for in vitro transcription and translation systems. The in vitro synthesized product will be enriched for the recombinant proteins and contain fewer growth factor contaminants than the conditioned media described below.

Synthesis of His-Tagged PIRP Proteins

Epitope tagging provides a powerful method for detecting recombinant proteins using epitope-specific antibodies or other binding partners and simplifies protein purification. For this effort, an oligonucleotide containing the 6×His-tag was used to fuse this sequence to the carboxyl terminus of the PIRP-M and PIRP-L cDNAs (SEQ ID NO:11 and 15, respectively) (FIG. 5A). These PCR products were cloned into the pCMV and pBSII vectors and sequenced and will be used for transfection assays; their effect on PIRP growth factor activity will be evaluated in colony formation assay (described below); it is expected that the His tag will not alter PIRP growth factor activity.

Cell Lines Expressing Tagged or Untagged PIRPs

Tables 7 and 8 below summarize results of expression of PIRP-L or PIRP-M, tagged or untagged in various transfected cell lines.

Table 9 provides similar information for stable Cell lines expressing GFP-tagged mutant PIRP-M and PIRP-L

TABLE 7
Expression Profiles for Stable Cell Lines
Expressing untagged PIRP-M and PIRP-L Proteins

| | | PIRP | | | PIRP |
|---|---|---|---|---|---|
| | PIRP mRNA | Protein | | PIRP mRNA | Protein |
| Cell Line* | Expressed | Expressed | Cell Line* | Expressed | Expressed |
| 293 PIRP-M | M | M (L?) | 293 PIRP-M-His | M-His | M-His |
| | | | | | (L-His ?) |
| 293 PIRP-M | M | M (L?) | 293 PIRP-M-His | M-His | M-His |
| SFM* adapted | | | SFM adapted | | (L-His ?) |
| 293 PIRP-L | L | L | 293 PIRP-L-His | L-His | L-His |
| 293 PIRP-L | L | L | 293 PIRP-L-His | L-His | L-His |
| SFM adapted | | | SFM adapted | | |
| 293 PIRP-M+PIRP-L | M & L | M & L | 293 PIRP-M-His + | M-His & | M-His & |
| | | | PIRP-L-His | L-His | L-His |
| 293 PIRP-M+PIRP-L | M & L | M & L | 293 PIRP-M-His + | M-His & | M-His & |
| SFM adapted | | | PIRP-L-His, SFM | L-His | L-His |
| | | | adapted | | |

*derived from 293 cells

TABLE 8
Expression Profiles for Stable Cell Lines
Expressing GFP-tagged PIRP-M and PIRP-L Proteins.

| | Full Length | | | | | |
|---|---|---|---|---|---|---|
| | PLP/DM20 | | PIRP-M | | PIRP-L | |
| Cell Lines | mRNA | Protein | mRNA | Protein | mRNA | Protein |
| 293 PLP-GFP | PLP | PLP | PLP | PIRP-M* | PLP | PIRP-L* |
| NT2 PLP-GFP | PLP | PLP | PLP | PIRP-M* | PLP | PIRP-L* |
| 293 DM20-GFP | DM20 | DM20 | DM20 | PIRP-M* | DM20 | PIRP-L* |
| NT2 DM20-GFP | DM20 | DM20 | DM20 | P1RP-M* | DM20 | PIRP-L* |
| 293 PLP-GFP M1L | PLP | — | PLP | PIRP-M* | PLP | . 8PIRP-L* |
| NT2 PLP-GFP M1L | PLP | — | PLP | ↑PIRP-M* | PLP | . 8PIRP-L* |
| 293 DM20-GFP M1L | DM20 | — | DM20 | ↑PIRP-M* | DM20 | . 8PIRP-L* |
| NT2 DM20-GEP M1L | DM20 | — | DM20 | ↑PIRP-M* | DM20 | . 8PIRP-L* |
| 293 PLP-GFP M1L/M205L | PLP | PLP | PLP | — | PLP | — |
| NT2 PLP-GFP M1L/M205L | PLP | PLP | PLP | — | PLP | — |
| 293 DM20-GFP | DM20 | — | DM20 | — | DM20 | . 8PIRP-L* |
| M1L/M205L | | | | | | |
| NT2 DM20-GFP | DM20 | — | DM20 | — | DM20 | . 8PIRP-L* |
| M1L/M205L | | | | | | |
| 293 PLP-GFP M1L/M234L | PLP | — | PLP | ↑PIRP-M* | PLP | — |
| 293 DM20-GFP | DM20 | — | DM20 | ↑PIRP-M* | DM20 | — |
| M1L/M234L | | | | | | |
| 293 PLP-GFP | PLP | — | PLP | — | PLP | — |
| M1L/M205L/M234L | | | | | | |
| 293 DM20-GFP | DM20 | — | DM20 | — | DM20 | — |
| M1L/M205L/M234L | | | | | | |
| 293 PLP-GFP Pro- | — | — | — | — | — | — |
| 293 DM20-GFP Pro- | — | — | — | — | — | — |

*Expressed during MG132 induced apoptosis only
↑Increased expression levels compared to M1+ constructs

TABLE 9
Expression Profiles of Stable Cell Lines Expressing GFP-tagged Mutant PIRP-M and PIRP-L Proteins

| | Full Length PLP/DM20 | | PIRP-M | | PIRP-L | |
|---|---|---|---|---|---|---|
| Cell Lines | mRNA | Protein | mRNA | Protein | mRNA | Protein |
| 293 JPLP-GFP | JPLP | JPLP | JPLP | PIRP-J* | JPLP | ? |
| 293 JDM20-GFP | JDM20 | JDM20 | JDM20 | PIRP-J* | JDM20 | ? |
| 293 JPLP-GFP M1L | JPLP | — | JPLP | ↑PIRP-J* | JPLP | ? |
| 293 JDM20-GFP M1L | JDM20 | — | JDM20 | ↑PIRP-J* | JDM20 | ? |
| NT2 JDM20-GFP M1L | JDM20 | — | JDM20 | ↑PIRP-J* | JDM20 | ? |
| 293 JPLP-GFP M1L/M205L | JPLP | — | JPLP | — | JPLP | ? |
| NT2 JPLP-GFP M1L/M205L | JPLP | — | JPLP | — | JPLP | ? |
| 293 JDM20-GFP M1L/M205L | JDM20 | — | JDM20 | — | JDM20 | ? |
| NT2 JDM20-GFP M1L/M205L | JDM20 | — | JDM20 | — | JDM20 | ? |
| 293 RshPLP M1L | RshPLP | — | RshPLP | ↑PIRP-M* | RshPLP | ↑PIRP-L* |
| 293 RshPLP M1L | RshDM20 | — | RshDM20 | ↑PIRP-M* | RshDM20 | ↑PIRP-L* |
| 293 PLP-GFP M1L/K268R | PLP | — | PLP | ↑PIRP-M* | PLP | ↑PIRP-L |
| | M1L/K268R | | M1L/K268R | K268R | M1L/K268R | K268R |
| 293 DM20-GFP M1L/K268R | DM20 | — | DM20 | ↑PIRP-M* | DM20 | ↑PIRP-L |
| | M1L/K268R | — | M1L/K268R | K268R | M1L/K268R | K268R |
| 293 PLP-GFP M1L/K275R | PLP | — | PLP | ↑PIRP-M* | PLP | ↑PIRP-L |
| | M1L/K275R | | M1L/K275R | K275R | M1L/K275R | K275R |
| 293 DM20-GFP M1L/K275R | DM20 | — | DM20 | ↑PIRP-M* | DM20 | ↑PIRP-L |
| | M1L/K275R | | M1L/K275R | K275R | M1L/K275R | K275R |
| 293 PLP-GFP M1L/K268R/K275R | PLP M1L/ | — | PLP M1L/ | ↑PIRP-M* | PLP M1L/ | ↑PIRP-L |
| | K268R/K275R | | K268R/K275R | K268R/K275R | K268R/K275R | K268R/K275R |
| 293 DM20-GFP M1L/K268R/K275R | DM20 M1L/ | — | DM20 M1L/ | ↑PIRP-M* | DM20 M1L/ | ↑PIRP-L |
| | K268R/K275R | | K268R/K275R | K268R/K275R | K268R/K275R | K268R/K275R |
| 293 PLP-GFP M1L/R272K | PLP | — | PLP | ↑PIRP-M* | PLP | ↑PIRP-L* |
| | M1L/R272K | | M1L/R275K | R275K | M1L/R275K | R275K |
| 293 DM20-GFP M1L/R272K | DM20 | — | DM20 | ↑PIRP-M* | DM20 | ↑PIRP-L* |
| | M1L/R272K | | M1L/R272K | R272K | M1L/R272K | R272K |
| NT2 PLP-GFP M1L/R272K | PLP | — | PLP | ↑PIRP-M* | PLP | ↑PIRP-L* |
| | M1L/R272K | | M1L/R275K | R275K | M1L/R275K | R275K |
| NT2 DM20-GFP M1L/R272K | DM20 | — | DM20 | ↑PIRP-M* | DM20 | ↑PIRP-L* |
| | M1L/R272K | | M1L/R272K | R272K | M1L/R272K | R272K |

*Expressed during MG132 induced apoptosis only
↑Increased expression levels compared to M1+ constructs

### EXAMPLE VI

### PIRPs as Growth Factors

Colony Formation Assay Establishes PIRP Protein Growth Factor Activity

By definition, a growth factor is any molecule which induces an increase in cell number in a given interval. If the growth factor acts through a receptor, signal transduction pathways evoke immediate responses mediated by, for example, protein kinase activity: long term responses are directed by gene expression and cellular remodeling. However, some growth factors (e.g., certain oncogene products) directly alter metabolic processes that regulate cellular growth and survival so that cells can be transformed, and as a result of expressing the growth factor, permanently exit cell cycle regulation.

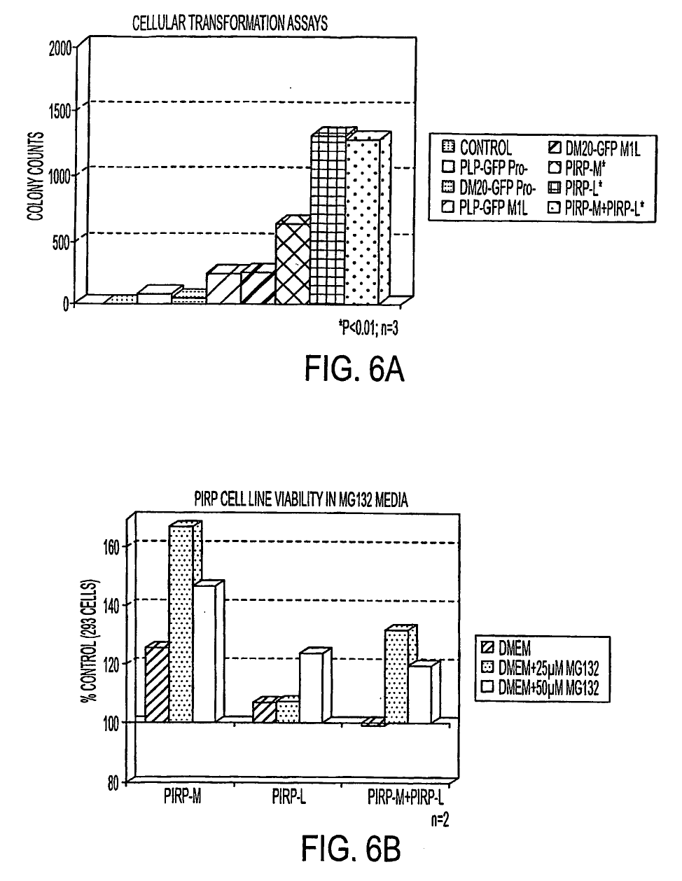

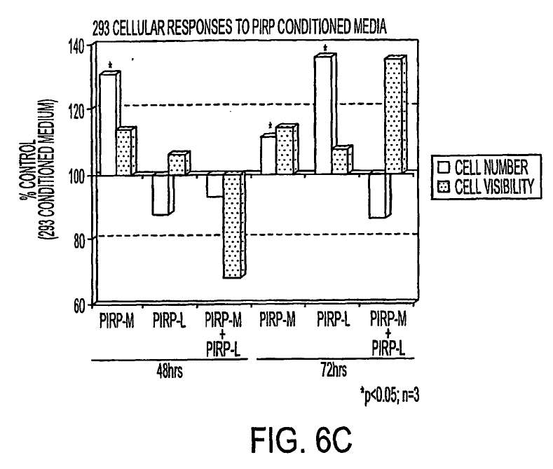

Simple systems cannot predict whether a PIRP acts as a receptor ligand or a direct regulator cellular proliferation. The present inventors employed a colony formation assay to test for oncogene-like activity in the PIRPs (FIG. 6A). As with the earlier PLP expression studies, these initial efforts used the human 293 cell line to examine cellular proliferation and colony formation. Standard calcium phosphate transfection and G418 selection procedures were used to generate colonies. To allow for variation in DNA purity, two independent DNA preparations were purified and transfected twice. PIRP expression vectors were transfected independently and as a combined DNA sample. Colonies were allowed to grow for 2 weeks prior to counting. For subsequent studies, the four colony plates produced for each PIRP vector assay were harvested and propagated as independent pooled cell samples.

A pBSII plasmid comprising PIRP-M cDNA was used as a DNA control since this vector lacks the G418-resistance gene. Compared to control DNA, the PLP-GFP Pro- and DM20-GFP Pro- vectors (see above) produced a modest increase in colony number (50-80-fold). These vectors include the PLP and DM20 ORFs, as well as G418^{R} but are missing the CMV promoter which prevents protein synthesis.

In contrast, the M1L PLP-GFP and M1L DM20-GFP vectors increased colony number 200-300-fold over controls, possibly due to endogenous PIRP translation in stressed and dying cells. However, these changes were minor compared to colony formation by PIRP-M- and PIRP-L-transfected cells (FIG. 6A). Colony numbers in cultures transfected with the PIRP-M vector increased by 600-700 fold over controls. Although these results were significant, an even greater response was observed in PIRP-L and PIRP-M/PIRP-L double transfectants where colony numbers increased 1200-1500-fold. These significant changes in colony number indicate that the PIRP genes transduce a selectable growth phenotype into 293 cells which results in higher colony numbers (two-tailed t-test; p<<0.01).

In addition, PIRP transformed cells exhibited a distinct trophic response and unusual colony morphology. Attempts to subclone single cell colonies from the pooled samples were unsuccessful due to the rapid migration of individual cells into aggregates. A similar trophic response has not been observed in any previous PLP-expressing cell line. Generally, 293 colonies expressing PLPs are well-ordered and flat with extended cellular borders composed of an occasional detached cell. In contrast, PIRP-expressing colonies were dense and raised with an unusual pattern of dispersed cells at their borders which appear to be migrating to or from the main colony. These migratory cells also tend to interact extensively via multiple projections that contact adjacent cells.

Another consisitent observation was the increased colony count in PIRP-L transfectants compared to PIRP-M transfectants. This statistically significant difference (p=0.02) suggests that the smaller PIRP-L protein exhibits a distinct enhanced colony formation phenotype vs PIRP-M activity.

Increased Viability of PIRP Expressing Cells During MG132-Induced Cellular Stress and Apoptosis

Although PIRP expression increased colony number, the biochemical mechanism of this response has yet to be defined. To examine whether PIRP protein expression increased cell viability during cellular stress and apoptosis, control 293 cells and PIRP-expressing cell lines were treated with sublethal (25 μM) and lethal (50 μM) doses of MG132. After 24 hrs, viable cells were counted (Trypan Blue exclusion) (FIG. 6B). This assay was performed twice on one set of PIRP-expressing cell lines; the results did not reach statistical significance, although viability tests of pooled cell lines may show different results.

Compared to 293 control cells, PIRP-M expression increased cellular viability in untreated (120% of control cell lines) and MG132-treated samples (145-165%). Such changes were not detected in untreated PIRP-L or PIRP-M/PIRP-L cell lines; however, a small increase in viability was observed in MG132-reated cells. These results suggest that the PIRP-M protein exhibits anti-apoptotic activity and increases cell viability in both “control” and stressed cells.

PIRP-M Transfected Cells Secrete Growth Factors

It is well established that primary neural cells secrete growth factors which can alter the morphology, proliferation rate and viability of other responsive cell types. In many cases, these growth factors can be detected by testing conditioned media (CM) (culture supernatants). To examine whether cell lines expressing the PIRPs secrete growth factors, medium (DMEM plus 10% fetal calf serum) was recovered from near-confluent cultures of 293 cells and PIRP-transfected cell lines after a 2 day incubation period. The CM was added to subconfluent, actively growing 293 cells for 48-72 hrs. Selection of such short intervals permitted testing for changes in cell number and viability prior to confluence. Although the cells were dispersed vigorously prior to plating, fluctuations in cell number and viability would likely reflect confluent regions of the culture surface which would manifest contact inhibition.

Cell number and viability increased in all cultures treated with PIRP CM (FIG. 6C). A statistically significant increase in cell number was observed at both time points (110-130% of 293 CM; Student's t-test; p<0.05; n=3). With one exception, PIRP-L and PIRP-M/PIRP-L CM tended to decrease cell number while increasing viability. It was concluded that PIRP-M expressing cells secrete a growth factor that is capable of increasing cell viability number.

PIRP Expressing Cells Rapidly Adapt to Growth in Serum Free Medium and Secrete Novel Proteins

The CM used to test for secreted PIRP growth factors was derived from DMEM and fetal calf serum (FCS). Given the abundance of proteins and growth factors in FCS, it is difficult detect any low abundance secreted protein. To simplify these studies, PIRP expressing cell lines and parental 293 cells were conditioned to grow in serum free medium (SFM; HYQSFM4HEK293 medium; Hyclone Industries, UT). In general, two independent 293 cultures were adapted to SFM; however, the parental cell line required an additional 2-3 weeks for adaptation compared to PIRP expressing cells. Furthermore, native 293 cells tended to grow in small (5 cells) cell clusters; whereas PIRP expressing cells routinely exhibited large (>25 cell) aggregates.

Conditioned media from 293, PIRP-M, PIRP-L and the PIRP-M/PIRP-L cell lines, as well as untreated SFM, were concentrated using Centricon filters that selectively retain small proteins. The media samples were applied to 20% SDS-PAGE gels and proteins detected by silver staining (FIG. 5B). As expected, the SFM contained no detectable protein. In contrast, a series of small proteins were detected in PIRP CM which were not present in control 293 CM (FIG. 5B). This suggests that small proteins are secreted from PIRP expressing cells.

### EXAMPLE VII

### Construction of PLP/DM20-M²⁰⁵-CAT and PLP/DM20-M234CAT Expression Vectors

An accepted method for studying IRES activity involves the use of artificial bicistronic constructs. The putative IRES sequence is inserted between two different reporter genes to produce a bicistronic mRNA, and the activity of both reporters is independently assayed. The upstream reporter activity reflects the efficiency of cap-dependent translation, while the downstream reporter activity measures the cap-independent (i.e. IRES driven) translation [132]. However, most of the IRES elements characterized by this method are found in the 5′ untranslated regions of their native mRNAs. The PLP IRES is one of the four known IRESs located in the coding sequence [142-144]. Thus, it is already placed in a “bicistronic” context, where the expression of the full size PLP and DM20 proteins reflects cap-dependent translation and expression of M²⁰⁵ and M²³⁴ initiation products measures cap-independent translation. Introducing two artificial reporters into this system may inactivate or alter the activity of the PLP IRES.

Fine mapping of the ribosomal binding site(s) and other cis-acting elements required the construction of a more sensitive reporter system than the PLP/DM20-GFP fusion constructs. An easily quantifiable CAT reporter was designed that facilitated detection of small changes in the IRES activity and allowed statistical evaluation of results. Previous analysis of IRES activity in JPLP/JDM20-GFP clones showed that altering the sequence downstream of M²⁰⁵ does not interfere with the stress-specific activation of this codon. Therefore, replacement of this sequence with any transgene should not affect M²⁰⁵ translational initiation.

To generate the PLP/DM20-M²⁰⁵-CAT fusion constructs, the Bam HI site in the PLP/DM20-GFP M1L plasmids was removed by cutting with Bam HI, fill-in with Klenow Large Fragment, and ligation. A new Bam HI site was introduced upstream of the M²⁰⁵ codon by inserting a CATCC sequence between the G and A of GAAUG. This was accomplished using the QuikChange protocol. This vector, which was termed the pIRES-M²⁰⁵ express plasmids, allowed the cloning of PCR fragments into the Met205 triplet via this unique BamHI site. To test this idea, these constructs were cut with Bam HI, blunted with Mung Bean Nuclease, recut with Not I, and ligated to the Not I digested CAT reporter fragment. This PCR fragment was generated using a set of primers that introduced an AAUG sequence at the 5′ end (where AUG is CAT initiation codon) and a Not I anchor at the 3′ end. Upon ligation, the GAAUG sequence was regenerated and the CAT AUG was placed in the M²⁰⁵ context.

A similar strategy was used to generate the PLP/DM20-M234-CAT fusion constructs. A unique Mlu I site was introduced upstream of M234 by inserting a CGCGT sequence between the first and second “A” of AAAUG. This vector was termed the pIRES-M234express plasmid. The CAT construct was produced as before substituting Mlu I for Bam HI.

Using the Met205/Met234 CAT Reporter Vectors to Functionally Map Proteolipid IRES Elements

The pIRES-Mexpress CAT reporter constructs were used for deletion mapping of the PLP IRES. The extent of large deletion clones is shown in FIG. 2A. The PLP/DM20-GFP Apa I deletion clones and the DM20-GFP Bgl II mutant #37 were produced using standard methodology. To make the Bgl II-exon 4 deletion mutant, the sequence between M²⁰⁵ codon and the 3′ end of PLP/DM20 gene were PCR amplified using primers with Bgl II and Sac II restriction anchors. The resulting PCR product was cloned into the Bgl II/Sac II digested DM20-GFP plasmid. To make the PLP/DM20-GFP exon 5 deletion mutants, the existing jimpy clones (JPLP-GFP and JDM20-GFP) were used to insert a missing G nucleotide using the QuikChange Site Directed Mutagenesis protocol (Stratagene). The new clones were referred to as jimpy G knock-in mutants.

The IRES activity of each deletion mutants was tested in stably transfected 293 cells in the absence and presence of proteasomal inhibitor MG132. MG132 induces apoptosis in 293 cells and causes a stress-specific switch in PLP/DM20 internal start codon selection. These studies defined the candidate region for fine mapping of the ribosomal binding site(s) and other cis-acting elements to exon 4 proximal sequences.

IRES Regulation of Transgene Translation During Apoptosis

Some of the strongest evidence that proteolipid gene mutations alter growth factor responses is provided by mutations which produce a severe disease phenotype. In complementation studies, transplantation of jp brain tissue into a normal animal improved jp OL longevity and myelination potential. However, normalization of jp OL cell number was only observed following transplantation of embryonic and not postnatal tissue [82,83]. A similar effect was also found in in vitro studies where jp OLs grown in standard medium mimicked the in vivo phenotype where cells could not produce large membrane sheets or maintain normal cell numbers. However, when grown in media conditioned by normal cerebral cells, these cells exhibited extensive membrane sheets and an increase in cell number [85]. Together, these studies suggest that a factor(s) is either absent or defective in the developing jp brain which can be supplemented by paracrine signals from normal tissue.

The jp mutation likely affects directly the proteolipid growth factor. When the wt PLP gene is introduced into the jp animal, neither the PLP transgene nor the DM20 homolog is capable of complementing the mutant phenotype [102]. Combining the two transgenes increased the number of myelinated axons but did not correct the myelin deficiency [102,103]. This inability to rescue the jp phenotype is attributed to a “dominant negative phenotype” associated with this mutation. It has been suggested that the mutant protein(s) prevent any increase in OL survival even in the presence of the endogenous and wildtype transgenes. It has been proposed that the mutant protein(s) are the primary cause of OL death by directly or indirectly affecting OLs, neighboring neural cells which are engaged in the production of trophic or survival factors, or cells which monitor the OL plasma membrane and mediate their destruction [102,103]. Therefore, it seems likely that a direct consequence of the jp mutation would be the elimination of a growth factor activity associated with the carboxyl terminus of the PLP/DM20 gene (ie. exons 5-7).

In a recent set of biochemical and gene expression studies, PLP/DM20 protein products were shown to directly regulate myelination and the survival of immature and differentiated OLs and neurons. It was shown that nonglial cell lines forced to express the wild type proteolipid proteins secreted a soluble factor that increased proliferation of both oligodendroglial and astroglial lineage cells [104-106]. In contrast, cells expressing mutant PLP and DM20 proteins (i.e. the jp, jp^{msd} and rsh proteins) failed to exhibit a similar proliferative effect [107]. Together these studies suggest that a C-terminal proteolipid peptide participates in signaling between OLs and neurons.

Therefore, the pIRES-Mexpress vectors were used to express the PIRP-J protein during apoptosis. These vectors allowed the regulated translation of this potentially toxic protein in normal and apoptotic cells. These studies were designed to determine whether the PIRP-J protein regulates the sensitivity of cells to apoptotic stimulation.

### EXAMPLE VIII

Growth Factor Control of Embryonic Stem Cell Differentiation

Recovery from CNS trauma is hindered by the apparent inability of the vertebrate CNS to regenerate lost cells, replace damaged myelin and re-establish neural connections. In many CNS disorders, including multiple sclerosis (MS), stroke and trauma, demyelination is important in the loss of neuronal function.¹⁹⁸ Functional recovery might be achieved if intact axons can be rapidly remyelinated prior to Wallerian degeneration, a notion that has provoked interest in stem cell therapy. Stem cells may be defined as pluripotent cells capable of indefinite replication in culture, self-maintenance, and differentiation into mature, post-mitotic cell types in response to extrinsic environmental cues in vivo. Embryonic mammals have organotypic embryonic stem cell (ESC). In adult animals, organ-resident stem cells are thought to replenish cell loss due to normal physiological turnover, as well as death produced by pathological insults. In adults, stem cells have been detected in a wide variety of tissues including the CNS, bone marrow, skeletal muscle, intestine, liver, pancreas, epidermis, peripheral nervous system and retina. The mammalian brain was long thought to exhibit low neuronal turnover rates, thus severely limiting regenerative capacity. However, recent studies have shown the presence in adult brain of neural stem cells (NSC) with a capacity to generate differentiated progeny in vitro and in vivo. [198]

In the intact adult nervous system, stem cell division is tightly regulated to prevent unwanted cellular proliferation. The number of mature myelinating OL's is maintained by apoptotic elimination of extra oligodendroglial cells that are not associated with axons. In rats, up to 50% of the newly differentiated OL's in the optic nerve die within 2-3 days of generation [204-207]. In the developing neocortex, 20% of premyelinating cells are lost between P7 and P21 and 37% are lost between P21 and P28 [124,208] (and similar levels of cell death are observed in the spinal cord between P2 and P8). This regulatory process limits the effectiveness of natural neural recovery.

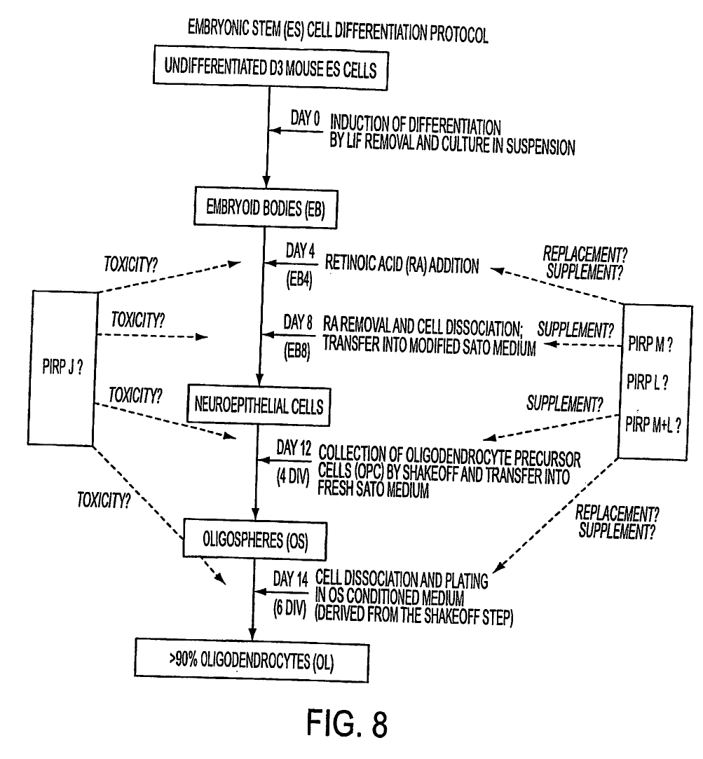

Various in vivo “delivery systems” have been developed to supplement the natural recovery systems and stimulate stem cell proliferation and differentiation. This includes providing growth factors to damaged brain regions. However, these efforts encountered obstacles when in vivo and in vitro lineage analyses discovered that NSC development proceeds through intermediate stages involving lineage-restricted progenitor cells that are committed to generation of either neurons (neuronal progenitor cells, NPC) or glial progenitors (GPC) that produce only astroglial or oligodendroglial cells. Therefore, normal neural development proceeds stepwise and can be monitored by the expression of stage-specific markers [209-211] For example, OL precursors (OLP) committed to OL development express early OL markers such as the A2B5 antigen, transferrin, the platelet derived growth factor a receptor (PDGFαR) and the proteolipid DM20 transcript [199-203]. Therefore, considerable effort has been made to define exogenous growth factors which specify NS development to glial progenitors (FIG. 8). These enriched glial cultures were then transplanted into demyelinated spinal cord and brain to define the parameters of remyelination. The present invention will identify growth factors which enhance NS cell commitment to glial development as an essential step toward in human therapeutic studies.

PIRP Protein Control of Stem Cell Development

Stem cell lines can serve as a source of cellular material for transplantation. The basic is that some diseases may be treatable by transplantation of a defined quantity of genetically characterized and potentially modified stem cells. Stem cells also provide a self-sustaining system for analyzing development and physiological mechanisms in post-mitotic cell types. The ability to manipulate and monitor cellular development has shown that pluripotent neural stem cells can differentiate into neurons, astrocytes and OLs and that commitment to specific differentiation pathways is dependent upon culture conditions [198].

At this time, a variety of methods have been described to differentiate embryonic stem (ES) and NS cells into neurons and glia [212-215]. In one study, addition of retinoic acid (RA) at 4 days results in stem cell commitment to neural lineage cells (FIG. 8). When dissociated and plated on adhesive substrates, cells differentiate into neurons, astrocytes and OLs. Mechanical manipulation and continued exposure to conditioned media results in the production of homogeneous cultures of OLs. These cells express terminally differentiated markers (such as the PLP protein) and myelinate axons in vitro and in vivo.

The PIRP-L, PIRP-M and PIRP-J proteins are tested for their effect on stem cell differentiation. A variety of studies will examine the activity of the endogenous PIRP proteins, as well as exogenous purified protein to affect stem cell commitment. To evaluate the impact of endogenous proteolipid proteins on stem cell differentiation, proteolipid-specific translational inhibitors are used to selectively inactivate protein synthesis. Proteolipid gene silencing employs either RNA interference (RNAi) [218-220] or antisense phosphorothioate-protected oligodeoxynucleotides (aODNs) [196] to generate double-stranded RNA structures which block translation and trigger ribonuclease degradation. These inhibitors are added at specific developmental stages to remove the endogenous PLP. To examine exogenous PIRP growth factor activity, purified proteins are tested and found to work at concentrations between about 1 and 10 pg/ml (the optimal dose previously described in PLP peptide studies of the proteolipid growth factor). PIRP proteins are found to supplement or replace the requirement for specific growth factors during the developmental protocol (FIG. 8).

### EXAMPLE IX

### Mixed Cell Cultures Suggest That the Proteolipid Gene Regulates Neuronal Viability

As previously described by the present inventors [108], mature OLs and neurons are dependent upon cooperative signaling systems to modulate their common survival. To further study how the proteolipid proteins contribute to these interactions, a cell culture system was used to examine the impact of PLP expression on neuronal viability. More specifically, co-cultures of dorsal root ganglia (DRG) neurons and 293 cells expressing the PLP and DM20 proteins were examined for changes in neuronal viability. In general, the DRG neurons in these cultures did not intimately associate with the proteolipid expressing cells. An occasional axon passed over proteolipid positive cells but no mixed cell aggregation or extensive membrane interactions were observed. This suggested that PLP expressing cell lines do not attract or intimately associate with DRG neurons. However, quantitative analysis revealed that only ˜50% of the DRG neurons survived growth with PLP expressing cells compared to control cultures and co-cultures containing DM20 expressing cells. DRG neurons in the PLP co-cultures exhibited nuclear degeneration, reduced membrane refractivity, shrunken somata, punctate tubulin staining and segmented axons, all indicators of neurons undergoing apoptotic cell death.

To determine whether the toxic PLP phenotype was dependent upon cell-cell contact, DRG neurons were cultured in CM prepared from PLP-expressing cells. Following the addition of CM, DRG neurons exhibited >2.5-fold decline in number when compared to control neurons grown in CM prepared from DM20 expressing or untransformed 293 cells (p<0.05, two-tailed t-test). Therefore, PLP CM appeared to contain a secreted factor which reduced DRG neuron viability at high concentrations. In this study, it was not shown whether this factor was produced from the PLP gene or induced in 293 cells by PLP protein expression.

In a second study, DRG neurons were grown with 293 Tet-On cells expressing the PLP protein. In contrast to earlier studies, the number of surviving DRG neurons increased 1.5-fold after Dox induction when compared to untreated DRG neurons, Dox treated DRG neurons or untreated DRG/PLP Tet-On co-cultures (P<0.05, two-tail t-test). Furthermore, neuronal aggregates with cell clusters containing 5 or more cells increased significantly and exhibited an increase in the number of axons which projected over long distances (>250 μm), thicker fascicle bundles and an increase in tubulin immunostaining. When PLP expression was examined, a maximal doxycycline (Dox) dosage was found to induce less than 50% of the PLP protein level in CMV 293 cell lines. It was suggested that lower PLP protein levels might contribute to neuronal survival or PLP protein expression induces the synthesis of neuronal pro-survival factors in 293 Tet-On cells.

In either case, these studies suggest that a secreted factor is produced by PLP expressing cells which regulates DRG neuronal viability. Since no evidence exists that the full-length PLP protein can be secreted from cells, any growth factor activity derived from the intact protein would require partial proteolytic cleavage to generate a bioactive peptide. Since PLP protein expression appears to both enhance and reduce neuronal viability, this may correlate with a proteolipid growth factor that enhances cell survival at low concentrations but adversely affects viability at higher doses. Or alternatively, PLP protein expression in different 293 backgrounds induces the production of distinct growth factors which exhibit positive and negative effects on DRG neuronal viability. *

Examining the Effect of the PIRP Proteins on Neuronal and Oligodendrocyte Viability

It has been suggested that remyelination after neural trauma recapitulates the entire molecular process of OL differentiation and myelinogenesis. Consistently, the onset of remyelination in MS and EAE is accompanied by increased expression of the embryonically preferred DM20 mRNA and select forms of the MBP transcript [109,110]. The extent of spontaneous recovery of EAE animals was correlated with increased expression of the DM20 mRNA, regardless of whether remission occurred after onset or after relapse. The reinduction of DM20 transcription is specific for the active phase of sustained remission and likely represents DM20 expression in OLPs [109], since DM20 transcript levels returned to low levels during long-term remission. This appears to implicate DM20-derived protein factors in this recovery process via glial precursor cells.

Given the unique commitment phase associated with OLPs, a DM20 derived secreted trophic factor (namely, a PIRP protein) should stimulate terminal OL differentiation, recruit OLs to damage sites, and/or increase the viability of the remyelinating OLs. It is expected that these effects are responsible for the positive in vivo results detected in these demyelinating disorders. Equally important would be the effect of OL-specific growth factors on axonal viability. Since axonal degeneration is the terminal effect of demyelinating diseases which prevents extensive repair by endogenous recovery systems, OL growth factors will also function to prevent cell damage and apoptotic death.

The present studies are designed to examine the effect of the PIRP proteins on OL and neuronal viability. Ex vivo cultures of enriched OLs and DRG neurons are treated with purified proteins and assayed for cell number and viability. These studies will show whether these proteins enhance or restrict the viability of cultured neural cells.

All the references cited herein are incorporated herein by reference in their entirety, whether specifically incorporated or not. Having now fully described this invention, it will be appreciated by those skilled in the art that the same can be performed within a wide range of equivalent parameters, concentrations, and conditions without departing from the spirit and scope of the invention and without undue experimentation.

### LITERATURE CITED

[1] Baumann N et al., (2001) Physiol Rev. 81:871-927
[2] Southwood C et al., (2001) Microsc Res Tech. 52:700-708
[3] Diehl H J et al., (1986) Proc Natl Acad Sci USA. 83:9807-9811
[4] Macklin W B et al., (1987) J Neurosci Res. 18:383-394
[5] Milner R J et al., (1985) Cell. 42:931-939
[6] Nadon N L et al., (1990) Development. 110:529-537
[7] Tosic M et al., (1994) J Neurochem. 63:2210-2216
[8] Baumgartner B G et al., (1999) Mamm Genome. 10:895-899
[9] Baumgartner B G et al., (2000) DNA Seq. 10:379-385
[10] Lees M B et al., (1983) Arch Biochem Biophys. 226:643-656
[11] Schliess F et al., (1991) Biol Chem Hoppe Seyler. 372:865-784
[12] Tohyama Y et al., (1999) Neurochem Res. 24:867-873
[13] Tohyama Y et al., (2000) Brain Res Mol Brain Res. 80:256-259
[14] Nave K A et al., (1987) Proc Natl Acad Sci USA. 84:5665-5669
[15] Bongarzone E R et al., (1999) J Neurosci. 19:8349-8357
[16] Afshari F S et al., (2001) J Neurosci Res. 66:37-45
[17] Janz R et al., (1993) Biol Chem Hoppe Seyler. 374:507-517
[18] Kim J G et al., (1992) Mol Cell Biol. 12:5632-5639
[19] Saluja I et al., (2001) Glia. 33:191-204
[20] Awatramani R et al., (2000) J Neurosci Res. 61:376-387
[21] Ikenaka K et al., (1992) J Neurochem. 58:2248-2253
[22] Timsit S et al., (1995) J Neurosci. 15:1012-1024
[23] Timsit S G et al., (1992) J Neurochem. 58:1172-1175
[24] Trapp B D et al., (1997) J Cell Biol. 137:459-468
[25] Campagnoni C W et al., (1992) J Neurosci Res. 33:148-155
[26] Pribyl T M et al., (1996a) J Neurosci Res. 45:812-819
[27] Pribyl T M et al., (1996b) J Neuroimmunol. 67:125-130
[28] Puckett C et al., (1987) J Neurosci Res. 18:511-518
[29] Kamholz J et al., (1992) J Neurosci Res. 31:231-244
[30] Griffiths I R et al., (1995) Neuropathol Appl Neurobiol. 21:97-110
[31] Nadon N L et al., (1997) Int J Dev Neurosci. 15:285-293
[32] Jolles J et al., (1979) Biochem Biophys Res Commun. 87:619-626
[33] Stoffel W et al., (1984) Proc Natl Acad Sci USA. 81:5012-5016
[34] Kahan I et al., (1985) Biochemistry. 24:538-544
[35] Trifilieff E et al., (1986) FEBS Lett. 198:235-239
[36] Konola J T et al., (1992) Glia. 5:112-121
[37] Sobel R A et al., (1994) J Neurosci Res. 37:36-43
[38] Greer J M et al., (1996) Neurochem Res. 21:431-440
[39] Gow A et al., (1997) J Neurosci. 17:181-189
[40] Popot J L et al., (1991) J Membr Biol. 120:233-246
[41] Weimbs T et al., (1992) Biochemistry. 31:12289-12296
[42) Weimbs T et al., (1994) Biochemistry. 33:10408-10415
[43] Smith R et al., (1984) J Neurochem. 42:306-313
[44] Brown F R 3rd et al., (1985) Neurosci Lett. 59:149-154
[45] Laidlaw D J et al., (1985) Eur Biophys J. 12:143-151
[46] Whikehart D R et al., (1973) J Neurochem. 20:1303-1315
[47] Wiggins R C et al., (1974) J Neurochem. 22:337-340
[48] Bizzozero O A et al., (1999) Neurochem Res. 24:269-267
[49] Messier A M et al., (2000) Neurochem Res. 25:449-455
[50] Southwood C M et al., (2002) Neuron. 36:585-596
[51] Gardner R G et al., (1999) EMBO J. 18:5994-6004
[52] Griffiths I R. (1996) Bioessays. 18:789-797
[53] Yool D A et al., (2000) Hum Mol Genet. 9:987-992
[54] Campagnoni A T et al., (2001) Brain Pathol. 11:74-91
[55] Garbern J et al., (1999) Arch Neurol. 56:1210-1214
[56] Cailloux F et al., (2000) Eur J Hum Genet. 8:837-845
[57] Ellis D et al., (1994) Nat Genet. 6:333-334
[58] Inoue K et al., (1999) Ann Neurol. 45:624-632
[59] Boison D et al., (1994) Proc Natl Acad Sci USA. 91:11709-11713
[60] Klugmann M et al., (1997) Neuron. 18:59-70
[61] Griffiths I et al., (1998) Science. 280:1610-1613
[62] Inoue Y et al., (1996) Neurosci Res. 25:161-172
[63] Readhead C et al., (1994) Neuron. 12:583-595
[64] Yamamoto T et al., (1998) Am J Med Genet. 74:439-440
[65] Komaki H et al., (1999) Pediatr Neurol. 20:309-311
[66] Kobayashi H et al., (1994) Nat Genet. 7:351-352
[67] Nave K A et al., (1986) Proc Natl Acad Sci USA. 83:9264-9268
[68] Hudson L D et al., (1987) Proc Natl Acad Sci USA. 84:1454-1458
[69] Duncan I D et al., (1989) Glia. 2:148-154
[70] Peyron F et al., (1997) J Neurosci Res. 50:190-201
[71] Skoff R P. (1976) Nature. 264:560-562
[72] Dupouey P et al., (1980) J Neurosci Res. 5:387-398
[73] Skoff R P. (1982) Brain Res. 248:19-31
[74] Ghandour M S et al., (1988) J Neurocytol. 17:485-498
[75] Williams W C 2nd et al., (1997) J Neurosci Res. 50:177-189
[76] Thomson C E et al., (1999) J Neurocytol. 28:207-221
[77] Knapp P E et al., (1986) J Neurosci. 6:2813-2822
[78] Vela J M et al., (1996) Brain Res. 12:134-142
[79] Privat A et al., (1981) Brain Res. 254:411-416
[80] Wu Q et al., (2000) J Neurosci. 20:2609-2617
[81] Cerghet M et al., (2001) J Neurocytol. 30:841-855
[82] Lachapelle F et al., (1992) Dev Neurosci. 14:105-113
[83] Lachapelle F et al., (1994) Neurochem Res. 19:1083-1090
[84] Bartlett W P et al., (1988) Glia. 1:253-259
[85] Feutz A C et al., (1995) J Neurocytol. 24:865-877
[86] Richardson W D et al., (1988) Cell. 53:309-319
[87] Raff M C et al., (1988) Nature. 333:562-565
[88] McMorris F A et al., (1988) J Neurosci Res. 21:199-209
[89] Canoll P D et al., (1996) Neuron. 17:229-243
[90] McKinnon R D et al., (1990) Neuron. 5:603-614
[91] McKinnon R D et al., (1993) Glia. 7:245-254
[92] Barres B A et al., (1994) Nature. 367:371-375
[93] Louis J C et al., (1993) Science. 259:689-692
[94] Robinson S et al., (1998) J Neurosci. 18:10457-10463
[95] McKinnon R D et al., (1993) J Cell Biol. 121:1397-1407
[96] Mayer M et al., (1994) Development. 120:143-153
[97] Park S K et al., (2001) Dev Neurosci. 23:327-337
[98] Feutz A C et al., (2001) Glia. 34:241-252
[99] Schneider A et al., (1992) Nature. 358:758-761
[100] Tosic M et al., (1994) J Neurochem. 63:2210-2216
[101] Readhead C et al., (1990) Behav Genet. 20:213-234
[102] Nadon N L et al., (1994) J Neurochem. 63:822-833
[103] Schneider A M et al., (1995) Proc Natl Acad Sci USA. 92:4447-4451
[104] Nakao J et al., (1995) J Neurochem. 64:2396-2403
[105] Knapp P E et al., (1999) Cell Death Differ. 6:136-145
[106] Yamada M et al., (1999) J Neurosci. 19:2143-2151
[107] Yamada M et al., (2001) Neurochem Res. 26:639-645
[108] Boucher S E et al., (2002) J Neurosci. 22:1772-1783
[109] Mathisen P M et al., (2001) J Neurosci Res. 64:542-551
[110] Capello E et al., (1997) Ann Neurol. 41:797-805
[111] Barnard R O et al., (1967) J Neurol sci. 5:441-455
[112] Giordana M T et al., (1981) Ital J Neurol Sci. 2:403-409
[113] Shankar S K et al., (1989) Neurosurgey. 25:982-986
[114] Khan O A et al., (1997) Neurology. 48:1330-1333
[115] Green A J et al., (2001) Mult Scler. 7:269-273
[116] Poncelet V et al., (2001) J Neuroradiol. 28:130-135
[117] Pal E et al., (2001) Eur J Neurol. 8:717-718
[118] Taricco M A et al., (2002) Arq Neuropsiquiatr. 60:475-477
[119] Werneck L C et al., (2002) Arq Neuropsiquiatr. 60:469-474
[120] Pestova T V et al., (2001) Proc Natl Acad Sci USA. 98:7029-7036
[121] Gray N K et al., (1998) Annu Rev Cell Dev Biol. 14:399-458
[122] Rhoads R E. (1999) J Biol Chem. 274:30337-30340
[123] Watkins S J et al., (2002) Br J Cancer. 86:1023-1027
[124] Marissen W E et al., (2000) Cell Death Differ. 7:1234-1243
[125] Marissen W E et al., (2000) J Biol Chem. 275:9314-9323
[126] Borman A M et al., (1997) RNA. 3:186-196
[127] Ohlmann T et al., (1996) EMBO J. 15:1371-1382
[128] Kozak M. (1989) J Cell Biol. 108:229-241
[129] Kozak M. (1991) Gene Expr. 1:111-115
[130] Kozak M. (1995) Proc Natl Acad Sci USA. 92:2662-2666
[131] Kozak M. (2000) Genomics. 70:396-406
[132] Hellen C U et al., (2001) Genes Dev. 15:1593-1612
[133] MartinezSalas E et al., (2001) J Gen Virol. 82:973-984
[134] Belsham G J et al., (1996) Microbiol Rev. 60:499-511
[135] Martinez-Salas E, et al., (2002) Biochimie. 84:775-763
[136] Lopez de Quinto S et al., (1999) Virology. 255:324-336
[137] Cao X et al., (1995) J Virol. 69:560-563
[138] Hinton T M et al., (2000) J Virol. 74:11708-11716
[139] Vagner S et al., (2001) EMBO Rep. 2:893-898
[140] Bonnal S et al., (2003) Nucleic Acids Res. 31:427-428
[141] Johannes G et al., (1999) Proc Natl Acad Sci USA. 96:13118-13123
[142] Cornelis S, et al., (2000) Mol Cell. 5:597-605
[143] Lauring A S et al., (2000) Mol Cell. 6:939-945
[144] Maier D et al., (2002) Proc Natl Acad Sci USA. 99:15480-15485
[145] Chappell S A, et al., (2000) Proc Natl Acad Sci USA. 97:1536-1541
[146] Sella O et al., (1999) Mol Cell Biol. 19:5429-5440
[147] Miskimins W K et al., (2001) Mol Cell Biol. 21:4960-4967
[148] Chappell S A et al., (2003) J Biol Chem. 278:33793-33800
[149] Hu M C et al., (1999) Proc Natl Acad Sci USA. 96:1339-1344
[150] Owens G C et al., (2001) Proc Natl Acad Sci USA. 98:1471-1476
[151] Bernstein J et al., (1997) J Biol Chem. 272:9356-9362
[152] Gan W et al., (1998) J Biol Chem. 273:5006-5012
[153] Le SY et al., (1992) Virology. 191:858-866
[154] Le SY et al., (1993) Nucleic Acids Res. 21:2445-2451
[155] Le SY et al., (1995) Gene. 154:137-143
[156] Scheper G C et al., (1994) FEBS Lett. 352:271-275
[157] Liu Z et al., (1999) Virology. 265:206-217
[158] Yang D et al., (2003) Virology. 305:31-43
[159] Le SY et al., (1997) Nucleic Acids Res. 25:362-369
[160] Hudder A et al., (2000) J Biol Chem. 275:34586-34591
[161] Millard S S et al., (2000) Mol Cell Biol. 20:5947-5959
[162] Pickering B M et al., (2003) Nucleic Acids Res. 31:639-646
[164] Mitchell S A et al., (2001) Mol Cell Biol. 21:3364-3374
[165] Mitchell S A et al., (2003) Mol Cell. 11:757-771
[166] Nanbru C et al., (1997) J Biol Client. 272:32061-32066
[167] Huez I et al., (1998) Mol Cell Biol. 18:6178-6190
[168] Johannes G et al., (1998) RNA. 4:1500-1513
[169] Le Quesne J P et al., (2001) J Mol Biol. 310:111-126
[170] Huez I et al., (2001) Mol Endocrinol. 15:2197-2210
[171] Negulescu D et al., (1998) J Biol Chem. 273:20109-20113
[172] Sachs A B (2000) Cell. 101:243-245
[173] Werner R. (2000) IUBMB Life. 50:173-176
[174] Pinkstaff J K et al., (2001) Proc Natl Acad Sci USA. 98:2770-2775
[175] Nevins T A et al., (2003) J Biol Chem. 278:3572-3579
[176] Holcik M et al., (2000) Trends Genet. 16:469-473
[177] Schiavi A et al., (1999) FEBS Lett. 464:118-122
[178] Dermietzel R et al., (1997) Glia. 20:101-114
[179] Friessen A J et al., (1997) J Neurosci Res. 50:373-382
[180] Stein I et al., (1998) Mol Cell Biol. 18:3112-3119
[181] Vagner S et al., (1995) Mol Cell Biol. 15:35-44
[182] Galy B et al., (1999) Cancer Res. 59:165-171
[183) Holcik M et al., (1999) Nat Cell Biol. 1:190-192
[184] Yang Q et al., (1997) Nucleic Acids Res. 25:2800-2807
[185] Fernandez J, et al., (2001) J Biol Chem. 276:12285-12291
[186] HenisKorenblit S et al., (2000) Mol Cell Biol. 20:496-506
[187] Stoneley M et al., (2000) Mol Cell Biol. 20:1162-1169
[188] Coldwell M J et al., (2000) Oncogene. 19:899-905
[189] Awatramani R et al., (1997) J Neurosci. 17:6657-6668
[190] Shao W et al., (2000) FEBS Lett. 473:363-369
[191] Kim J G et al., (1998) Mol Cell Neurosci. 12:119-140
[192] Linington C et al., (1990) J Neurochem. 54:1354-1359
[193] Stern S et al., (1988) Methods Enzymol. 164:481-489
[194] Bain G et al., (1995) Dev Biol. 168:342-357
[195] Liu S et al., (2000) Proc Natl Acad Sci USA. 97:6126-6131
[196] Yang X et al., (1997) J Neurosci. 17:2056-2070
[197] Landry C F et al., (1997) Cancer Res. 57:4098-4104
[198] Gottlieb D I. (2002) Annu Rev Neurosci. 25:381-407
[199] Dickinson P J et al., (1996) Neuropathol Appl Neurobiol. 22:188-198
[200] Espinosa de los Monteros A et al., (1988) Int J Dev Neurosci. 6:167-175
[201] Farrer R G et al., (1999) J Neurosci Res. 43:315-330
[201] Nishiyama A et al., (1996) J Neurosci Res. 43:315-330
[202] Mallon B S et al., (2002) J Neurosci. 22:876-885
[203] Wilson H C et al., (2003) Glia. 44:153-165
[204] Barres B A et al., (1992) Cell. 70:31-46
[205] Burne J F et al., (1996) J Neurosci. 16:2064-2073
[206] Raff M C et al., (1993) Science. 262:695-700
[207] Raff M C. (1996) Cell. 86:173-175
[208] De Louw A J et al., (2002) Glia. 37:89-91
[209] Blesch A et al., (2002) Brain Res Bull. 57:833-838
[210] Okano H. (2002) J Neurosci Res. 69:698-707
[211] Du Y et al., (2002) J Neurosci Res. 68:647-654
[212] Brustle O et al., (1999) Science. 285:754-756
[213] Fraichard A et al., (1995) J Cell Sci. 108:3181-3188
[214] Strubing C et al., (1995) Mech Dev. 53:275-287
[215] Deacon T et al., (1998) Exp Neurol. 149:28-41
[216] Mathews D H et al., (1999) J Mol Biol. 288:911-940
[217] Zucker M et al., (1999) RNA Biochemistry and Biotechnology. 1143
[218] Cheng J C et al., (2003) Mol Genet Metab. 80:121-128
[219] Wilson J A et al., (2003) Curr Opin Mol Ther. 5:389-396
[220] Lavery K S et al., (2003) Curr Opin Drug Discov Devel. 6:561-569
[221] Levi-Montalcini R et al., (1964) Int Ser Monogr Oral Biol. 21:129-141
[222] Van Straaten F et al., (1983) Proc Natl Acad Sci USA. 80:3183-3187
[223] Folch-Pi, J. et al. (1972). Ann N Y Acad Sci. 195: 86-107.
[224] Folch, J. et al. (1951). J Biol Chem. 191: 807-817.
[225] Wolfgram, F. (1966). J Neurochem. 13: 461-470.
[226] Gow, A. (1997). J Neurosci Res. 50: 659-664.
[227] Yan, Y. et al. (1993). Neuron. 11: 423-431.
[228] Stecca, B. et al. (2000). J Neurosci. 20: 4002-4010.
[229] Eng, L. F. et al. (1968). Biochemistry. 7: 4455-4465.
[230] Nussbaum, J. L. et al. (1983). Cell Tissue Res. 234: 559-59.
[231] Schwob, V. S. et al. (1985). J Neurochem. 45: 559-571.
[232] Sinoway, M. P., et al. (1994). J Neurosci Res. 37: 551-562.
[233] Gow, A. et al. (1996). Nat Genet. 13: 422-428.
[234] McLaughlin, M. et al. (2002). Glia. 39: 31-36.
[235] Houbre, D. et al. (1990). Biochim Biophys Acta. 1029: 136-142.
[236] Horvath, L. I. et al. (1990). Biochemistry. 29: 2635-2638.
[237] Yamaguchi, Y. et al. (1996). J Biol Chem. 271: 27838-27846.
[238] Gudz, T. I. et al. (2002). J Neurosci. 22: 7398-7407.
[239] Lin, L. F. et al. (1982). Proc Natl Acad Sci USA. 79: 941-945.
[240] Lin, L. F. et al. (1984). Neurochem Res. 9: 1515-1522.
[241] Benjamins, J. A. et al. (1994). Neurochem Res. 19: 1013-1022.
[242] Benjamins, J. A. et al. (1989). J Neurochem. 53: 279-286.
[243] Vouyiouklis, D. A. et al. (2000). J Neurochem. 74: 940-948.
[244] Duncan, I. D. et al. (1987). Proc Natl Acad Sci USA. 84: 6287-6291.
[245] Duncan, I. D. et al. (1987). Brain Res. 402: 168-172.
[246] Boison, D. et al. (1995). J Neurosci. 15: 5502-5513.
[247] Rosenbluth, J. et al. (1996). J Comp Neurol. 371: 336-344.
[248] Helynck, G. et al. (1983). Eur J Biochem. 133: 689-695.
[249] de Cozar, M. et al. (1987). Biochem Int. 14: 833-841.
[250] Diaz, R. S. et al. (1990). et al. J Neurochem. 55: 1304-1309.
[251] Hoy, M. et al. (2002). Proc Natl Acad Sci USA. 99: 6773-6777.
[252] Kagawa, T. et al. (1994). J Neurochem. 62: 1887-1893.
[253] Skoff. R. P. et al. (1995). J Comp Neurol. 355: 124-133.
[254] Edgar, J. M. et al. (2002). J Cell Biol. 158: 719-729.
[255] Vermeesch, et al. (1990). Dev Neurosci. 12: 303-315.
[256] Gow, A. et al. (1998). J Cell Biol. 140: 925-934
[257] Griffiths, I. R. et al. (1990). J Neurocytol. 19: 273-283.
[258] Mitchell, L. S. et al. (1992). J Neurosci Res. 33: 205-217.
[259] Nadon, N. L. et al. (1998). Dev Neurosci. 20: 533-539.
[260] Boespflug-Tanguy, O. et al. (1994). Am J Hum Genet. 55: 461-467.
[261] Saugier-Veber, P. et al. (1994). Nat Genet. 6: 257-262.
[262] Seitelberger, F. (1995). Brain Pathol. 5: 267-273.
[263] Garbern, J. Y. (Updated Aug. 16, 2002). PLP-related disorders. In: Gene Reviews at Gene Tests-Gene Clinincs Medical Genetics Information Resource (database online). Available at http://www.geneclinics.org or http://www.genetests.org. Accessed Sep. 12, 2002.
[264] Wang, P. J. et al. (1997). Pediatr Neurol. 17: 125-128.
[265] Sistermans, E. A. et al. (1998). Neurology. 50: 1749-1754.
[266] Woodward, K. et al. (1998). Am J Hum Genet. 64: 207-217.
[267] Mimault, C. et al. (1999). Am J Hum Genet. 65: 360-369.
[268] Knapp, P. E. (1996). Dev Neurosci. 18: 297-308.
[269] Vela, J. M. et al. (1998). Brain Res Brain Res Rev. 26: 29-42.
[270] Chan, D. S. et al. (1974). Biochemistry. 13: 2704-2712.
[271] Sorg, B. J. et al. (1986). J Neurochem. 46: 379-387.
[272] Lepage, P. et al. (1986). Biochimie. 68: 669-686.
[273] Bizzozero, O. A. et al. (2002). J Neurochem. 81: 636-645.
[274] Pyronnet, S. et al. (2000). Mol Cell. 5: 607-616.
[275] Miller, D. L. et al. (1998). FEBS Lett. 434: 417-420.
[276] Zeller, N. K. et al. (1989). J Mol Neurosci. 1: 139-149.
[277] Beesley, J. S. et al. (2001). J Neurosci Res. 64: 371-379.
[278] Calver, A. R. et al. (1998). Neuron. 20: 869-882.
[279] Barres, B. A. et al. (1993). Development. 118: 283-295.
[280] Barres, B. A. et al. (1993). Nature. 361: 258-260.
[281] Barres, B. A. et al. (1999). J Cell Biol. 147: 1123-1128.
[282] Casaccia-Bonnefil, P. (2000). Glia. 29: 124-135.
[283] Spassky, N. et al. (1998). J Neurosci. 18: 8331-8343.
[284] Spassky, N. et al. (2000). Glia. 29: 143-148.
[285] Yool, D. A. et al. (2001). J Neurosci Res. 63: 151-164.
[286] Lucchinetti, C. F. et al. (1996). Brain Pathol. 6: 259-274.
[287] Dowling, P. et al. (1997). J Neurol Sci. 149: 1-11.
[288] Jung, M. et al. (1996). J Neurosci. 16: 7920-7929.
[289] Williams, R. T. et al. (2001). Biochem J. 357: 673-685.

1. An isolated, recombinant polypeptide molecule comprising a first amino acid sequence which is a fragment of a native proteolipid protein having a wild type or mutant sequence as compared with the native sequence of said proteolipid protein, and optionally comprising a second amino acid sequence fused in frame thereto to create a fusion polypeptide, which first polypeptide is encoded by an mRNA having an Internal Ribosome Entry Site ((IRES) wherein translation of the mRNA initiates at said IRES, such that the N-terminal amino acid residue of said first polypeptide corresponds to an internal residue of said proteolipid protein.

2. The polypeptide of claim 1 wherein the proteolipid protein is human PLP/DM20.

3. The polypeptide or of claim 1 selected from the group consisting of:
(a) PIRP-M, having the amino acid sequence SEQ ID NO:6;
(b) PIRP-L, having the amino acid sequence SEQ ID NO:8;
(c) a fusion polypeptide of (a) or (b) wherein said second amino acid sequence encodes a naturally fluorescent protein or peptide;
(d) a His-tagged fusion polypeptide of PIRP-M having the amino acid sequence SEQ ID NO:12;
(e) a His-tagged fusion polypeptide of PIRP-L having the amino acid sequence SEQ ID NO:16; and
(f) PIRP-J having a mutant sequence compared to said proteolipid protein, the sequence of said PIRP-J being SEQ ID NO:18, or a human homologue thereof.

4. The polypeptide of claim 3 which is PIRP-M having the amino acid sequence SEQ ID NO:6

5. The polypeptide of claim 3 which is PIRP-L, having the amino acid sequence SEQ ID NO:8.

6. The polypeptide of claim 3 which is PIRP-J having the amino acid sequence SEQ ID NO:18.

7. The fusion polypeptide of claim 3 wherein said fluorescent protein is yellow or green fluorescent protein (GFP) or a fluorescent homologue thereof.

8. The His-tagged fusion polypeptide of claim 3 having the sequence SEQ ID NO:12.

9. The His-tagged fusion polypeptide of claim 3 having the sequence SEQ ID NO:16.

10. An isolated nucleic acid encoding the polypeptide of claim 1, the mutant sequence thereof, or the fusion polypeptide thereof.

11. The nucleic acid of claim 10 which is a DNA molecule.

12. The nucleic acid of claim 10 which is an RNA molecule.

13. The nucleic acid of claim 10 wherein the proteolipid protein is human PLP/DM20.

14. The nucleic acid of claim 10 encoding a polypeptide or fusion polypeptide selected from the group consisting of:
(a) PIRP-M, having the amino acid sequence SEQ ID NO:6;
(b) PIRP-L, having the amino acid sequence SEQ ID NO:8;
(c) a fusion polypeptide of (a) or (b) wherein said second amino acid sequence encodes a naturally fluorescent protein or peptide;
(d) a His-tagged fusion polypeptide of PIRP-M having the amino acid sequence SEQ ID NO:12;
(e) a His-tagged fusion polypeptide of PIRP-L having the amino acid sequence SEQ ID NO:16; and
(f) PIRP-J having a mutant sequence compared to said proteolipid protein, the sequence of said PIRP-J being SEQ ID NO:18, or a human homologue thereof.

15. The nucleic acid of claim 14 which encodes PIRP-M and has a nucleotide sequence SEQ ID NO:5 or SEQ ID NO:9.

16. The nucleic acid of claim 14 which encodes PIRP-L and has a nucleotide sequence SEQ ID NO:7 or SEQ ID NO:13.

17. The nucleic acid of claim 14 which encodes PIRP-J and has a nucleotide sequence SEQ ID NO:17.

18. The nucleic acid of claim 14 which encodes said His-tagged fusion polypeptide of PIRP-M, which nucleic acid has a nucleotide sequence SEQ ID NO:11;

19. The nucleic acid of claim 14 which encodes said His-tagged fusion polypeptide of PIRP-L, which nucleic acid has a nucleotide sequence SEQ ID NO:15;

20. The nucleic acid of claim 14 which encodes said fusion polypeptide wherein said second amino acid sequence encodes a naturally fluorescent protein or peptide.

21. The nucleic acid of claim 20 wherein said fluorescent protein is yellow or green fluorescent protein (GFP) or a fluorescent homologue thereof.

22. The nucleic acid of claim 10 operatively linked to a promoter.

23. The nucleic acid of claim 22, wherein the promoter is one which is expressed in a mammalian cell.

24. The nucleic acid of claim 23 wherein said mammalian cell is a neuronal cell, a glial cell or a stem cell.

25. The nucleic acid of claim 24 wherein said glial cell is an oligodendrocyte.

26. The nucleic acid of claim 24 wherein the stem cell is a neural stem cell, an oligodendrocyte progenitor cell, an embryonic stem cell or a hemopoietic stem cell.

27. A vector comprising the nucleic acid of any of claim 10.

28. The vector of claim 27, selected from the group consisting of PLP-GFP/DM20-GFP; PLP-GFP/DM20-GFP Tet-On; PLP-GFP/DM20-GFP M1L; PLP-GFP/DM20-GFP M1L/M205L; PLP-GFP/DM20-GFP M1L/M234L; PLP-GFP/DM20-GFP M1L/M205L/M234L; PLP-GFP/DM20-GFP Pro-; JPLP-GFP/JDM20-GFP; JPLP-GFP/JDM20-GFP M1L; JPLP-GFP/JDM20-GFP M1L/M205L; RshPLP-GFP/RshDM20-GFP M1L; PLP-GFP/DM20-GFP M1L/K268R; PLP-GFP/DM20-GFP M1L/K275R; PLP-GFP/DM20-GFP M1L/K268R/K275R; and PLP-GFP/DM20-GFP M1L/R272K

29. An expression vector or cassette comprising the nucleic acid of claim 10 operatively linked to
(a) a promoter; and
(b) optionally, additional regulatory sequences that regulate expression of said nucleic acid in a eukaryotic cell.

30. The expression vector or cassette of claim 27 comprising a vector selected from the group consisting of pCMV; pEGFP-N1; pEYFP-N1; pEGFP-Tet-On; pBluescript II KS+; and pET-14b.

31. The expression vector or cassette of claim 28 elected from the group consisting of 205M-CMV/234M-CMV; 205M-His-CMV/234M-His-CMV; 205M-BsKS+/234M-BsKS+; 205M-His-BsKS+/234M-His-BsKS+; and 205M-ET-14b/234M-ET-14b.

32. A cell which has been modified to comprise the nucleic acid of claim 10.

33. The cell of claim 32 which is a mammalian cell.

34. A cell which has been modified to comprise the vector of claim 27.

35. A cell which has been modified to comprise the vector or expression cassette of claim 31.

36. The cell of claim 35 which expresses said nucleic acid.

37. The cell of claim 36 which is mammalian cell.

38. The cell of claim 37 wherein said mammalian cell is a neuronal cell, a glial cell or a stem cell.

39. The cell of claim 38 wherein said glial cell is an oligodendrocyte.

40. The cells of claim 38 wherein the stem cell is a neural stem cell, an oligodendrocyte progenitor cell, an embryonic stem cell or a hemopoietic stem cell.

41. A pharmaceutical composition, comprising:
(a) pharmaceutically acceptable excipient in combination with
(b) the polypeptide of claim 1.

42. A pharmaceutical composition, comprising:
(a) pharmaceutically acceptable excipient in combination with
(b) the nucleic acid of claim 23.

43. A pharmaceutical composition, comprising:
(a) pharmaceutically acceptable excipient in combination with
(b) the expression vector or cassette of claim 29;

44. A pharmaceutical composition, comprising:
(a) pharmaceutically acceptable excipient in combination with
(b) the cell of claim 33.

45. (canceled)

46. A method for stimulating oligodendroglial cells or Schwann cells and promoting remyelination, comprising providing to said cells an effective amount of the polypeptide of claim 4 or a functional derivative thereof, thereby stimulating said cells and promoting remyelination.

47. The method of claim 46 that is carried out in vivo in a mammalian subject in need of remyelination.

48. A method of treating a demyelinating or dysmyelinating disease or disorder in a mammalian subject, comprising administering to said subject
(i) the polypeptide of claim 4 or a functional derivative thereof, or
(ii) a pharmaceutical composition comprising said polypeptide or functional derivative, thereby treating said disease or disorder.

49. The method of claim 48, wherein the disease or disorder is multiple sclerosis, closed head trauma associated with Parkinson's-like symptoms, hypoxic ischemia, or spinal cord trauma.

50. A method for stimulating oligodendroglial cells or Schwann cells and promoting remyelination in a subject, comprising administering to a subject in need of remyelination an effective amount of the cells of claim 32 which have been modified by said nucleic acid that
(i) has a nucleotide sequence SEQ ID NO:5 or SEQ ID NO:9; or
(ii) encodes a polypeptide having the amino acid sequence SEQ ID NO:6, thereby promoting said remyelination.

51. A method of treating a demyelinating or dysmyelinating disease or disorder in a mammalian subject, comprising administering to said subject the cells of claim 32 which have been modified by said nucleic acid that
(i) has a nucleotide sequence SEQ ID NO:5 or SEQ ID NO:9; or
(ii) encodes a polypeptide having the amino acid sequence SEQ ID NO:6, thereby treating said disease or disorder.

52. A method of stimulating neural stem cell survival and promoting differentiation or maturation of said cells along the oligodendrocyte pathway, comprising providing to said neural stem cells an effective amount of the polypeptide of claim 4 or a functional derivative thereof.

53. A method for stimulating proliferation of oligodendrocytes and/or oligodendrocyte precursors, comprising providing to said oligodendrocytes and/or precursors an effective amount of the polypeptide of claim 4 or a functional derivative thereof.

54. A method of protecting oligodendrocytes from apoptotic death comprising providing to oligodendrocytes an effective amount of the polypeptide of claim 4 or a functional derivative thereof.

55. A method for treating a disease or disorder in which one or more of oligodendrocytic (a) differentiation, (b) maturation, (c) proliferation, and (d) inhibition of cell death is palliative or curative for said disease or disorder, comprising administering to a subject in need of such treatment an effective amount of
(i) the polypeptide of claim 4 or a functional derivative thereof, or
(ii) a pharmaceutical composition comprising said polypeptide or functional derivative, thereby treating said disease or disorder.

56. A method for treating a disease or disorder in which one or more of oligodendrocytic (a) differentiation, (b) maturation, (c) proliferation, and (d) inhibition of cell death is palliative or curative for said disease or disorder, comprising administering to a subject in need of such treatment an effective amount of the cells of claim 32 which have been modified by said nucleic acid that
(i) has a nucleotide sequence SEQ ID NO:5 or SEQ ID NO:9; or
(ii) encodes a polypeptide having the amino acid sequence SEQ ID NO:6, thereby treating said disease or disorder.

57. A method for regulating or inhibiting the production or action of PLP/DM20 or of PIRP-M polypeptide under conditions in which said PLP/DM20 or PIRP-M is pathogenically produced in cells or in a subject, comprising providing to the cells or to the subject an effective amount of the polypeptide of claim 5 or a functional derivative thereof.

58. The method of claim 57 wherein the polypeptide or functional derivative is administered to a subject with oligodendroglioma or a benign glial tumor

59. A method for regulating or inhibiting the production or action of PLP/DM20 or of PIRP-M polypeptide under conditions in which said PLP/DM20 or PIRP-M is pathogenically produced in cells or in a subject, comprising providing to the cells or to the subject an effective amount of the cells of claim 32 which have been modified by said nucleic acid that
(i) has a nucleotide sequence SEQ ID NO:7 or SEQ ID NO:13, or
(ii) encodes a polypeptide comprising an amino acid sequence SEQ ID NO:8.

60. The method of claim 59 wherein the cells being provided are administered to a subject with oligodendroglioma or a benign glial tumor.
